(19) United States
(12) Patent Application Publication
(10) Pub. No.: US 2025/0160951 A1
(43) Pub. Date: May 22, 2025

(54) SYSTEMS, DEVICES AND METHODS FOR LASER-ASSISTED TARGETED INTRACORPOREAL THERAPY

(71) Applicant: Light Matter Interaction Inc., Etobicoke (CA)

(72) Inventors: R. J. DWAYNE MILLER, Mississauga (CA); Darren Kraemer, Toronto (CA); Paroshat Vafaei, Toronto (CA); Samuel John Virtue, Toronto (CA)

(21) Appl. No.: 19/029,963

(22) Filed: Jan. 17, 2025

Related U.S. Application Data
(63) Continuation-in-part of application No. PCT/CA2024/051323, filed on Oct. 4, 2024.
(60) Provisional application No. 63/542,404, filed on Oct. 4, 2023.
(60) Provisional application No. 63/571,203, filed on Mar. 28, 2024.

(51) Int. Cl. A61B 18/24; A61B 10/02; A61B 18/00; A61B 18/20; A61B 18/22

(52) U.S. Cl. CPC A61B 18/24; A61B 10/0283; A61B 2018/00184; A61B 2018/00404; A61B 2018/00446; A61B 2018/00625; A61B 2018/00702; A61B 2018/00714; A61B 2018/2005; A61B 2018/2205; A61B 2018/2255

(57) ABSTRACT

Systems and methods are disclosed that facilitate the local therapy of tissue within the body. In some example embodiments, infrared laser pulses are locally delivered, via optical fiber, to an intracorporal target tissue region and are provided with pulse conditions suitable for causing local tissue disruption and liquification, leading to fine tissue disruption, tissue homogenization, and removal of vasculature and interstitial fluid channels, and enabling passage of the distal tip of the optical fiber into the target tissue region without substantial tissue deformation and damage along a preferred surgical pathway. When optical fiber emitting such pulses is employed to penetrate tumor tissue, the resulting reduction of interstitial fluid pressure facilitates the subsequent injection of a drug into the tumor, enabling the drug to remain localized within the tumor with reduced diffusion. The tumor disruption and subsequent drug delivery may be performed using an integrated optical and fluidic delivery device.

### CROSS-REFERENCE TO RELATED APPLICATION

This application is a continuation-in-part application which claims priority to International Patent Application No. PCT/CA2024/051323, titled “SYSTEMS, DEVICES AND METHODS FOR LASER-ASSISTED TARGETED INTRACORPOREAL THERAPY” and filed on Oct. 4, 2024, the entire contents of which is incorporated herein by reference, and PCT/CA2024/051323 application claims priority to U.S. Provisional Patent Application No. 63/542,404, titled “PULSED INFRARED LASER (PIRL) BASED DRUG DELIVERY SYSTEM TO OVERCOME THE BLOOD-BRAIN BARRIER” and filed on Oct. 4, 2023, the entire contents of which is incorporated herein by reference, and PCT/CA2024/051323 application also claims priority to U.S. Provisional Patent Application No. 63/571,203, titled “SYSTEMS, DEVICES AND METHODS FOR LASER-ASSISTED TARGETED TISSUE THERAPY” and filed on Mar. 28, 2024, the entire contents of which is incorporated herein by reference.

### BACKGROUND

The present disclosure relates to systems and methods for delivering local therapy to a target tissue region inside of the body.

Challenges in delivering therapeutic agents directly to areas within the body such as the brain or solid tumors with minimal tissue damage to the surroundings have long been limitations in medical procedures. The use of conventional needles often results in undesirable tracks of inflammation or damage, undermining treatment efficacy and patient recovery, especially in delicate tissues or are unable to effectively deliver drugs effectively. Furthermore, fine needle biopsy is hindered by the size of needle and amount of tissue that can be extracted while also causing damage to the nearby tissue. For example, fine needle biopsy of the brain is often avoided for this reason

Cancer treatments involve a plurality of treatment regimes. One of the most often used treatments is the use of anticancer drugs in a procedure known as chemo treatments. These drugs are administered systemically due to the challenges of directly injecting drugs at cancer sites. This requirement means that cancer drugs must be highly selective for uptake by rapidly dividing cells, or other morphological features, that are unique to cancer cells (or other diseased tissue) over normal cells.

Unfortunately, in solid tumors, several factors inhibit the homogenous distribution of systemic drugs, including limited regional blood flow to the tumor, permeability of the tumor vasculature, structural barriers imposed by perivascular tumor cells and extracellular matrix, and intratumorally pressure. These impediments to the delivery of systemic drugs to tumor can be understood from FIG. 1 of Kobayashi et al. (Kobayashi H, Watanabe R, Choyke PL. Improving Conventional Enhanced Permeability and Retention (EPR) Effects; What Is the Appropriate Target?, Theranostics 2014; 4 (1): 81-89. doi: 10.7150/thno.7193), which shows the local tumor environment and morphology. The impact of the local tumor morphology and pressure can greatly limit the number of drugs that can be successfully delivered. Moreover, even in the case of highly selective drugs, there are still major problems with the side effects of chemotherapy. The quality of life for people enduring chemotherapy can be extremely poor and can require life support in many cases.

As shown in FIG. 1A of Böckelmann et al. (Clemens Böckelmann & Udo Schumacher (2019): Targeting tumor interstitial fluid pressure: will it yield novel successful therapies for solid tumors?, Expert Opinion on Therapeutic Targets, DOI: 10.1080/14728222.2019.1702974), in normal tissue, capillaries are covered by pericytes, the surrounding interstitium contains loose extracellular matrix (ECM) produced by local fibroblasts and penetrated by lymphatic vessels. Hydrostatic and colloid osmotic pressures illustrate the opposing forces involved in filtration and transvascular fluid exchange; in steady state net outward filtration pressure is around 1 mm Hg. The distribution of solute molecules by diffusion covers for a distance of up to 100 μm from the capillary (dashed line). Convection flow reaches out of capillaries, through the interstitial space, and into the lymphatic vessels and is based upon a positive pressure gradient between the capillary and the lymphatic vessels.

However, as shown in FIG. 1B of Böckelmann et al., in tumor tissue, high interstitial fluid pressure (IFP) reverses the situation resulting in a net increase in the pressure within the interior of the tumor, ultimately hampering convection driven transport of solute molecules. The pressure gradient is now reaching from the center toward the periphery thereby enabling an opposite fluid flow. This leads to a gradient of decreasing oxygen (O₂), nutrient, and drug uptake. IFP is a product of a leaky tumor vasculature, missing lymph vessels, and a denser and stiffer ECM produced by cancer-associated fibroblasts (CAFs).

Another important challenge for drug delivery is to penetrate or circumvent the blood-brain barrier. The Blood-brain Barrier (blood-brain barrier) protects the brain form infection but also serves as a barrier to the uptake of drugs into the brain having a size larger than about 400 Daltons (Da). The treatment of mental illnesses, have to date been limited to the small molecule regime comparable. Small molecule being defined as 400 Da or smaller. Molecules in this category typically have fewer than 20 atoms with mass greater than 12 Da per atom, with typical examples of brain relative active compounds such as the neurotransmitter serotonin. This mass range is an order of magnitude or more, smaller than other biomolecules such as neuropeptides that are involved in long term regulation of brain activity. This barrier completely eliminates the key mass range of potential therapeutic drugs for mental illness, which is one of the most pervasive medical conditions afflicting people of all ages. The brain is such a complex organ that there are many causes that can disrupt normal thinking processes. Changes in brain chemistry can lead to depression, debilitating anxiety, destructive manic behaviour, loss of memory retrieval, and learning disabilities. There are also triggered events in the brain that lead to epilepsy and other nervous system-based controls of body functions as occurs with Parkinson disease. One of the most debilitating diseases is Alzheimer's disease that currently is incurable and leads to loss of memories and ultimately to loss of the key memories associated with life experiences and functions that identify an individual a person. To put the scale of the problem in perspective 60% of university students currently suffer some form of mental illness, from anxiety to depression with over 30% requiring clinical treatment with drugs selected on a hit or miss basis according to self-reported symptoms.

The present situation is further exasperated by the opioid crisis, which targets the brain to still pain receptors and leads to run away addiction due to changes in brain chemistry. The current palette of drugs for treating mental illness is limited to masses of 400 Da (400 g/mole or 400 atomic mass units) that are able to pass the blood-brain barrier (BBB). This limits drug development to molecules on the scale of caffeine to try to find serotonin receptors, inhibitors etc. to reset the brain chemistry. Alzheimer's disease alone affects nearly the entire population to some degree at the later stages of life and is an intolerable state for the person and family with enormous costs in terms of end-of-life care. It is known from mice studies that Alzheimer's is largely connected to excessive tau protein that is needed for brain function but builds up in synaptic junctions and limits brain connectivity. Treatment regimes using tau antibodies have been shown in mouse models of Alzheimer's to completely reverse the disease and restore the brain's plasticity to recall and to learn. The problem is that tau antibodies have masses of over 10 KDa. Massive, toxic, doses (>100× estimated needed to affect brain function) are needed to achieve an effect as these antibodies have very little transmission through the blood-brain barrier. Despite these limitations, 3 new antibody based drugs have approved by the FDA for treating Alzheimer's disease. This regime requires monthly transfusions with only minor delay of a few months in offsetting the onset of dementia based on statistics for the group monitored with blind controls. The fact that these biological drugs work despite very little transmission through the blood-brain barrier indicates the importance of getting past the blood-brain barrier for developing optimal treatment regimes.

### SUMMARY

Systems and methods are disclosed that facilitate the local therapy of tissue within the body. In some example embodiments, infrared laser pulses are locally delivered, via optical fiber, to an intracorporal target tissue region and are provided with pulse conditions suitable for causing local tissue disruption and liquification, leading to fine tissue disruption, tissue homogenization, and removal of vasculature and interstitial fluid channels, and enabling passage of the distal tip of the optical fiber into the target tissue region without substantial tissue deformation and damage along a preferred surgical pathway. When an optical fiber emitting such pulses is employed to penetrate tumor tissue, the resulting reduction of interstitial fluid pressure facilitates the subsequent injection of a drug into the tumor, enabling the drug to remain localized within the tumor with reduced diffusion. The tumor disruption and subsequent drug delivery may be performed using an integrated optical and fluidic delivery device.

Accordingly, in one aspect, there is provided a system capable of performing laser-assisted tissue disruption, the system comprising:
a pulsed infrared laser source configured to generate infrared laser pulses;
an intracorporeal laser pulse delivery assembly comprising:
   an elongate conduit comprising an inner lumen and an elongate distal portion configured for intracorporeal insertion and having a diameter of less than 1 mm, the elongate distal portion having a distal opening in fluid communication with the inner lumen, the elongate conduit further comprising a proximal port, the proximal port being in fluidic communication with the inner lumen;
   an optical fiber having a proximal end coupled to the pulsed infrared laser source such that the infrared laser pulses are delivered through the optical fiber, the optical fiber extending through the elongate conduit; and
   a pump mechanism in fluid communication with the proximal port;
the pulsed infrared laser source being configured such that the infrared laser pulses have:
   a wavelength selected such that absorption by a laser-irradiated tissue volume residing beyond a distal end of the optical fiber, following intracorporeal insertion of the elongate distal portion, is predominantly due to excitation of vibrational modes of one or more constituents of the laser-irradiated tissue volume;
   a pulse duration that is shorter than a first time duration required for thermal diffusion out of the laser-irradiated tissue volume and shorter than a second time duration required for a thermally driven expansion of the laser-irradiated tissue volume;
   a pulse fluence and the pulse duration resulting in a peak pulse intensity below a threshold for ionization-driven tissue disruption to occur within the laser-irradiated tissue volume; and
   wherein the pulse fluence is sufficiently high to cause local tissue disruption and liquification of the laser-irradiated tissue volume.

In one example implementation of the system, the elongate conduit further comprises a proximal valve, and wherein the optical fiber passes through the proximal valve into the elongate conduit and extends through the elongate conduit, the proximal valve being configurable to form a fluidic seal with the optical fiber while permitting longitudinal movement of the optical fiber, wherein an outer diameter of the optical fiber is less than a diameter of the inner lumen, such that the proximal port is in fluid communication with the distal opening; wherein the intracorporeal laser pulse delivery assembly further comprises a distal beam expansion optical element coupled to the distal end of the optical fiber, such that the distal beam expansion optical element is mechanically supported by the optical fiber and resides beyond the distal opening of the elongate conduit, and such that the infrared laser pulses propagate from the optical fiber into the distal beam expansion optical element, and expand within the distal beam expansion optical element to substantially fill a distal forward-facing surface of the distal beam expansion optical element; the distal beam expansion optical element having a proximal portion configured to close and seal the distal opening of the elongate conduit when the optical fiber is retracted through the proximal valve, and configured to facilitate fluidic communication between the inner lumen and an external region beyond the distal opening when the optical fiber is extended through the proximal valve and the distal beam expansion optical element is moved distalward relative to the distal opening, the distal beam expansion optical element and the distal opening thereby forming a distal valve.

In one example implementation of the system, the distal forward-facing surface is curved to facilitate reduction in a frictional force during intracorporeal insertion of the elongate distal portion.

In one example implementation of the system, the intracorporeal laser pulse delivery assembly is further configured such that:
the elongate conduit comprises a proximal seal;
the elongate distal portion is a sheath extending distalward from a proximal portion of the elongate conduit, the inner lumen extending through the sheath to a distal opening of the sheath;
the optical fiber passes through the proximal seal, into the proximal portion of the elongate conduit, and extends through the sheath, such that the distal end of the optical fiber resides at or distally adjacent to a distal end of the sheath; and the sheath has a tapered profile such that a distal portion of the sheath is closer to an outer surface of the optical fiber than a proximal portion of the sheath;
wherein the pump mechanism is operable to dispense a fluid from the distal end of the sheath.

The distal portion of the sheath may contact and apply a passive compressive force to the outer surface of the optical fiber, thereby forming a distal valve that is passively closed via the passive compressive force, and wherein the pump mechanism is configured to apply sufficient pressure to overcome the passive compressive force to facilitate dispensing of the fluid through the distal valve.

In one example implementation of the system, the intracorporeal laser pulse delivery assembly further comprises:
a rigid body;
a syringe comprising a syringe barrel and a movable plunger having a plunger seal, the syringe barrel and the plunger seal defining an inner chamber;
wherein a proximal portion of the optical fiber is supported by a rigid elongate support, the rigid elongate support being rigidly supported relative to the syringe barrel by the rigid body, the rigid elongate support passing through the plunger seal, into the inner chamber, wherein the plunger seal is configured to form a fluidic seal with an outer surface of the rigid elongate support and an inner surface of the syringe barrel while the movable plunger is moved relative to the rigid elongate support; and wherein the elongate conduit extends from a distal end of the syringe barrel, such that the inner chamber is in fluid communication with the inner lumen of the elongate conduit through the proximal port of the elongate conduit, and wherein the optical fiber extends from a distal end of the rigid elongate support, through the inner chamber, and into the elongate conduit;
wherein the movable plunger, when actuated, is capable of dispensing a fluid from the distal end of the elongate distal portion of the elongate conduit.

In one example implementation of the system, the elongate distal portion is a microcannula suitable for incorporeal insertion.

In one example implementation of the system, the elongate distal portion is a microcatheter suitable for intravascular insertion and navigation.

The intracorporeal laser pulse delivery assembly may further include a microcannula, the elongate distal portion of the elongate conduit being retractable within the microcannula to facilitate intracorporeal insertion of the microcannula during delivery of the infrared laser pulses.

The system may further include a steering mechanism for steering the microcatheter when the microcatheter is extended from the microcannula.

In one example implementation of the system the pump mechanism is in fluid communication with a pharmaceutical fluid, and wherein the pump mechanism is controllable to facilitate direct injection of the pharmaceutical fluid after performing local tissue disruption and liquification of a laser-irradiated tissue volume.

In another aspect, there is provided a method of delivering a pharmaceutical fluid to an intracorporeal target site, the method comprising:
providing the system as described above, such that the pharmaceutical fluid resides within at least a distal portion of the inner lumen;
controlling the pulsed infrared laser source to perform local tissue disruption and liquification while positioning the distal end of the elongate distal portion proximal to or within the intracorporeal target site; and
controlling the pump mechanism to dispense the pharmaceutical fluid from the distal opening.

In one example implementation of the method, the intracorporeal target site resides within a brain of a subject, and wherein the elongate distal portion is a microcannula suitable for incorporeal insertion, and wherein the pulsed infrared laser source is controlled to perform local tissue disruption and liquification to facilitate insertion of the microcannula through a skull of a subject and positioning of a distal end of the microcannula within the brain of the subject.

In one example implementation of the method, the pulsed infrared laser source is controlled to perform local tissue disruption and liquification while passing through vessel walls of a cranial vessel as the distal end of the microcannula is directed to the intracorporeal target site within the brain.

In one example implementation of the method, the elongate distal portion is a microcatheter suitable for intravascular insertion and navigation, and wherein positioning the distal end of the microcatheter proximal to or within the intracorporeal target site comprises positioning a distal end of the microcatheter is adjacent to a vessel wall of a cranial vessel within a brain and controlling the pulsed infrared laser source to perform local tissue disruption and liquification of the vessel wall.

The method may further include extending the distal end of the elongate distal portion through the vessel wall to position the distal end of the microcatheter proximal to or within the intracorporeal target site.

In one example implementation of the method, the distal end of the elongate distal portion is positioned to adjacent to the vessel wall at a vascular bend in the absence of steering of the elongate distal portion.

In one example implementation of the method, the elongate distal portion is steerable according to a steering mechanism, and wherein the distal end of the elongate distal portion is steered to position the distal end of the elongate distal portion adjacent to the vessel wall.

In one example implementation of the method, a delivery catheter is employed to facilitate positioning of the distal end of the elongate distal portion within the cranial vessel.

In one example implementation of the method, the intracorporeal laser pulse delivery assembly further comprises a microcannula, the elongate distal portion of the elongate conduit being retractable within the microcannula, and the microcatheter is inserted to the cranial vessel by:
retracting the microcatheter within the microcannula, such that the distal end of the optical fiber resides at or distally adjacent to a distal end of the microcannula;
while delivering the infrared laser pulses, performing intracranial insertion of the microcannula and positioning the distal end of the microcannula within the cranial vessel; and
extending the microcatheter from the microcannula to position a distal end of the microcatheter adjacent to the vessel wall.

In one example implementation of the method, the vessel wall comprises a blood-brain barrier.

In another aspect, there is provided a method of delivering a pharmaceutical fluid to an intracorporeal target site, the method comprising:
providing the system as described above, including the distal beam expansion optical element, such that the pharmaceutical fluid resides within at least a distal portion of the inner lumen;
with the distal valve residing in a closed state, controlling the pulsed infrared laser source to perform local tissue disruption and liquification while positioning the distal end of the elongate distal portion proximal to or within the intracorporeal target site;
extending the optical fiber to move the distal beam expansion optical element away from contact with the distal opening to open the distal valve; and
controlling the pump mechanism to dispense the pharmaceutical fluid from the distal opening.

In one example implementation of the method, closure of the distal valve is facilitated, at least in part, by the application of a negative pressure by the pump mechanism to the inner lumen.

In one example implementation of the method, opening of the distal valve is facilitated, at least in part, by the application of a positive pressure by the pump mechanism to the inner lumen.

In another aspect, there is provided a method of performing biopsy at an intracorporeal target site, the method comprising:
providing the system as described above, including the distal beam expansion optical element;
retracting the optical fiber to contact the distal beam expansion optical element with the distal opening and close the distal valve;
controlling the pulsed infrared laser source to perform local tissue disruption and liquification while inserting the elongate distal portion into the body and positioning the distal end of the elongate distal portion proximal to or within the intracorporeal target site;
extending the optical fiber to extend the distal beam expansion optical element away from contact with the distal opening and open the distal valve; and
controlling the pump mechanism to aspirate tissue residing proximal to the distal opening.

In one example implementation of the method, closure of the distal valve is facilitated, at least in part, by the application of a negative pressure by the pump mechanism to the inner lumen.

In one example implementation of the method, the proximal valve is capable of locking a position of the optical fiber relative to the elongate conduit, the method further comprising, actuating the proximal valve to lock the position of the optical fiber relative to the elongate conduit to maintain the distal valve in an open configuration during aspiration of the tissue.

In another aspect, there is provided a system capable of performing laser-assisted thermal tissue disruption, the system comprising:
a pulsed laser source configured to generate a laser pulse or burst of laser pulses;
an optical fiber having a proximal end coupled to the pulsed laser source;
an optically absorbing material coated on a distal end of the optical fiber;
wherein the laser pulse or burst of laser pulses is delivered from the pulsed laser source with an energy such that the optically absorbing material is heated to a temperature between below its melting point over a time duration that is shorter a thermal diffusion timescale of the optically absorbing material when the distal end of the optical fiber is placed in water;
wherein the laser pulse or burst of laser pulses is provided with sufficient energy to cause vaporization of a volume of water residing adjacent to the distal end of the optical fiber; and
wherein the optically absorbing material is provided with a sufficient thickness to facilitate absorption of the laser pulse or burst of laser pulses.

In one example implementation of the system, the optically absorbing material is selected such that at least a portion of the optically absorbing material remains adhered to the distal end of the optical fiber after delivery of the laser pulse or burst of laser pulses.

In another aspect, there is provided a method of performing laser-assisted tissue disruption, the method comprising:
providing the system as described above;
inserting the optical fiber into a subject and positioning the distal end of the optical fiber proximal to or within an intracorporeal target site; and
controlling the pulsed laser source to deliver the laser pulse, such that a tissue volume residing proximal to the distal end of the optical fiber is thermally vaporized or emulsified into a liquid phase.

In another aspect, there is provided a fine needle delivery system capable of performing laser-assisted tissue disruption, the needle system comprising:
a pulsed infrared laser source configured to generate infrared laser pulses; and
a needle assembly comprising:
   an elongate conduit having a proximal end, an inner lumen, and an elongate distal portion configured for intracorporeal insertion and having a diameter of less than 1 mm, the elongate distal portion having a distal opening in fluid communication with the inner lumen, the elongate conduit further comprising a proximal valve a proximal port, the proximal port being in fluidic communication with the inner lumen;
   an optical fiber having a proximal end coupled to the pulsed infrared laser source such that the infrared laser pulses are delivered through the optical fiber, the optical fiber passing through the proximal valve into the elongate conduit and extending through the elongate conduit, the proximal valve being configurable to form a fluidic seal with the optical fiber while permitting longitudinal movement of the optical fiber, wherein an outer diameter of the optical fiber is less than a diameter of the inner lumen, such that the proximal port is in fluid communication with the distal opening;
   a distal beam expansion optical element coupled to a distal end of the optical fiber, such that the distal beam expansion optical element is mechanically supported by the optical fiber and resides beyond the distal opening of the elongate conduit, and such that the infrared laser pulses propagate from the optical fiber into the distal beam expansion optical element, and expand within the distal beam expansion optical element to substantially fill a distal forward-facing surface of the distal beam expansion optical element;
   the distal beam expansion optical element having a proximal portion configured to close and seal the distal opening of the elongate conduit when the optical fiber is retracted through the proximal valve, and configured to facilitate fluidic communication between the inner lumen and an external region beyond the distal opening when the optical fiber is extended through the proximal valve and the distal beam expansion optical element is moved distalward relative to the distal opening, the distal beam expansion optical element and the distal opening thereby forming a distal valve; and
a pump mechanism in fluid communication with the proximal port;
the pulsed infrared laser source being configured such that the infrared laser pulses have:
   a wavelength selected such that absorption by a laser-irradiated tissue volume residing beyond the distal forward-facing surface of the distal beam expansion optical element is predominantly due to excitation of vibrational modes of one or more constituents of the laser-irradiated tissue volume;
   a pulse duration that is shorter than a first time duration required for thermal diffusion out of the laser-irradiated tissue volume and shorter than a second time duration required for a thermally driven expansion of the laser-irradiated tissue volume;
   a pulse fluence and the pulse duration resulting in a peak pulse intensity below a threshold for ionization-driven tissue disruption to occur within the laser-irradiated tissue volume; and
   wherein the pulse fluence is sufficiently high to cause local tissue disruption and liquification of the laser-irradiated tissue volume; and
wherein the distal forward-facing surface of the distal beam expansion optical element is sufficiently large such that a lateral extent of the laser-irradiated tissue volume residing beyond the distal beam expansion optical element exceeds a diameter of a distal end of the elongate distal portion of the elongate conduit, thereby facilitating further insertion of the elongate distal portion into the laser-irradiated tissue volume.

In some example implementations of the system, the distal forward-facing surface is curved to facilitate a reduction in a frictional force during intracorporeal insertion of the elongate distal portion.

In another aspect, there is provided a method of delivering a pharmaceutical fluid to an intracorporeal target site via a fine needle, the method comprising:
providing the system described above such that the pharmaceutical fluid resides within at least a distal portion of the inner lumen;
with the distal valve residing in a closed state, controlling the pulsed infrared laser source to perform local tissue disruption and liquification while performing intracorporeal insertion of the elongate distal portion and positioning the distal end of the elongate distal portion proximal to or within the intracorporeal target site;
extending the optical fiber to move the distal beam expansion optical element away from contact with the distal opening to open the distal valve; and
controlling the pump mechanism to dispense the pharmaceutical fluid from the distal opening.

In some example implementations of the method, closure of the distal valve is facilitated, at least in part, by the application of a negative pressure by the pump mechanism to the inner lumen.

In some example implementations of the opening of the distal valve is facilitated, at least in part, by the application of a positive pressure by the pump mechanism to the inner lumen.

In another aspect, there is provided a method of performing needle biopsy at an intracorporeal target site, the method comprising:
providing the system as described above;
retracting the optical fiber to contact the distal beam expansion optical element with the distal opening and close the distal valve;
controlling the pulsed infrared laser source to perform local tissue disruption and liquification while inserting the elongate distal portion into the body and positioning the distal end of the elongate distal portion proximal to or within the intracorporeal target site;
extending the optical fiber to extend the distal beam expansion optical element away from contact with the distal opening and open the distal valve; and
controlling the pump mechanism to aspirate tissue residing proximal to the distal opening.

In some example implementations of the method, closure of the distal valve is facilitated, at least in part, by the application of a negative pressure by the pump mechanism to the inner lumen.

In some example implementations of the method, the proximal valve is capable of locking a position of the optical fiber relative to the elongate conduit, the method further comprising, actuating the proximal valve to lock the position of the optical fiber relative to the elongate conduit to maintain the distal valve in an open configuration during aspiration of the tissue.

In another aspect, there is provided a needle system capable of performing laser-assisted tissue disruption, the needle system comprising:
a pulsed infrared laser source configured to generate infrared laser pulses; and
a needle assembly comprising:
   an elongate conduit having a proximal end, an inner lumen, and a distal opening in fluid communication with the inner lumen, the elongate conduit further comprising a proximal valve and a proximal port, the proximal port being in fluidic communication with the inner lumen;
   a sheath having a proximal portion overlapping with and secured to an outer surface of the elongate conduit near a distal end of the elongate conduit, the sheath extending distalward from the distal end of the elongate conduit, the sheath having an inner lumen and a distal opening, wherein the inner lumen of the sheath is in fluid communication with the inner lumen of the elongate conduit;
   an optical fiber having a proximal end coupled to the pulsed infrared laser source such that the infrared laser pulses are delivered through the optical fiber, the optical fiber passing through the proximal valve into the elongate conduit and extending through the elongate conduit and the sheath, such that a distal end of the optical fiber resides at or distally adjacent to a distal end of the sheath;
   the proximal valve being configurable to form a fluidic seal with the optical fiber, wherein an outer diameter of the optical fiber is less than a diameter of the inner lumen of the elongate conduit, such that the proximal port is in fluid communication with the distal opening of the elongate conduit; and
   the sheath having a tapered profile such that a distal portion of the sheath contacts and applies a passive compressive force to an outer surface of the optical fiber, thereby forming a distal valve that is passively closed via the passive compressive force; and
a pump mechanism in fluid communication with the proximal port, the pump mechanism being capable of dispensing a liquid through the distal valve via application of sufficient pressure to overcome the passive compressive force;
the pulsed infrared laser source being configured such that the infrared laser pulses have:
   a wavelength selected such that absorption by a laser-irradiated tissue volume residing beyond the distal end of the optical fiber is predominantly due to excitation of vibrational modes of one or more constituents of the laser-irradiated tissue volume;
   a pulse duration that is shorter than a first time duration required for thermal diffusion out of the laser-irradiated tissue volume and shorter than a second time duration required for a thermally driven expansion of the laser-irradiated tissue volume;
   a pulse fluence and the pulse duration resulting in a peak pulse intensity below a threshold for ionization-driven tissue disruption to occur within the laser-irradiated tissue volume; and
   wherein the pulse fluence is sufficiently high to cause local tissue disruption and liquification of the laser-irradiated tissue volume.

In another aspect, there is provided a method of delivering a pharmaceutical fluid to an intracorporeal target site, the method comprising:
providing the system described above such that the pharmaceutical fluid resides within at least a distal portion of the inner lumen of the elongate conduit;
controlling the pulsed infrared laser source to perform local tissue disruption and liquification while performing intracorporeal insertion of the sheath and the optical fiber and positioning the distal end of the optical fiber proximal to or within the intracorporeal target site; and
controlling the pump mechanism to dispense the pharmaceutical fluid, such that the pump mechanism provides sufficient pressure to overcome the passive compressive force and open of the distal valve and the pharmaceutical fluid is dispensed within a region proximal to the distal end of the optical fiber.

In another aspect, there is provided a needle system capable of performing laser-assisted tissue disruption, the needle system comprising:
a pulsed infrared laser source configured to generate infrared laser pulses; and
a needle assembly comprising:
   a rigid body;
   a syringe comprising a syringe barrel and a movable plunger, the syringe barrel and the movable plunger defining an inner chamber;
   a sheath having a proximal portion overlapping with and secured to an outer surface of a distal end of the syringe barrel, the sheath extending distalward from the distal end of the syringe barrel, the sheath having an inner lumen and a distal opening, wherein the inner lumen of the sheath is in fluid communication with the inner lumen of the syringe; and
an optical fiber having a proximal end coupled to the pulsed infrared laser source such that the infrared laser pulses are delivered through the optical fiber, wherein a proximal portion of the optical fiber is supported by a rigid elongate support, the rigid elongate support being rigidly supported relative to the syringe barrel by the rigid body, the rigid elongate support passing through a moving seal defined within the movable plunger, into the inner chamber, wherein the moving seal is configured to form a fluidic seal with the rigid elongate support while the movable plunger is moved relative to the rigid elongate support;
   wherein a distal portion of the optical fiber extends from a distal end of the rigid elongate support, through the inner chamber and the sheath, such that a distal end of the optical fiber resides at or distally adjacent to a distal end of the sheath;
   the sheath having a tapered profile such that a distal portion of the sheath contacts and applies a passive compressive force to an outer surface of the optical fiber, thereby forming a distal valve that is passively closed via the passive compressive force; and
wherein the movable plunger, when actuated, is capable of dispensing a liquid through the distal valve via application of sufficient pressure to overcome the passive compressive force;
the pulsed infrared laser source being configured such that the infrared laser pulses have:
   a wavelength selected such that absorption by a laser-irradiated tissue volume residing beyond the distal end of the optical fiber is predominantly due to excitation of vibrational modes of one or more constituents of the laser-irradiated tissue volume;
   a pulse duration that is shorter than a first time duration required for thermal diffusion out of the laser-irradiated tissue volume and shorter than a second time duration required for a thermally driven expansion of the laser-irradiated tissue volume;
   a pulse fluence and the pulse duration resulting in a peak pulse intensity below a threshold for ionization-driven tissue disruption to occur within the laser-irradiated tissue volume; and
   wherein the pulse fluence is sufficiently high to cause local tissue disruption and liquification of the laser-irradiated tissue volume.

In another aspect, there is provided a method of delivering a pharmaceutical fluid to an intracorporeal target site, the method comprising:
providing the system of described above such that pharmaceutical fluid resides within the inner chamber;
   controlling the pulsed infrared laser source to perform local tissue disruption and liquification while performing intracorporeal insertion of the sheath and the optical fiber and positioning the distal end of the optical fiber proximal to or within the intracorporeal target site; and
   actuating the movable plunger to dispense the pharmaceutical fluid, such that the movable plunger provides sufficient pressure to overcome the passive compressive force and open of the distal valve and the pharmaceutical fluid is dispensed within a region proximal to the distal end of the optical fiber.

The present disclosure also solves the problem in introducing drugs into the brain or other organs of any arbitrary mass as long as the drug can be solubilized in a biologically safe liquid, taken to be normally aqueous based, but could also involve slow-release powder suspensions. The basic concept is to create a hole so small in a particular blood vessel within the brain that it is too small to allow blood to leak out of the blood vessel; while allowing injection of a drug under pressure through this opening. It is imperative to do this procedure with the least amount of damage to brain tissue as possible. The procedure specifically exploits the very strong absorption of water in the infrared region to selective excite water in the very tissue comprising the vessels and dense perineurium and endothelial cells making up the blood-brain barrier. By using a Pulsed InfraRed Laser (PIRL) with pulses shorter than the thermal diffusion time for the heating volume, it is possible using fiber delivery of the laser energy to locally disrupt tissue on the 10-100 micron scale comparable to the dimensions of single cells to completely confine the laser energy to the targeted tissue. The procedure creates the opening in the fully elastic regime so that upon exit there is very little or no bleeding. As a secondary measure, in the event of bleeding, the opening can be closed with the same fiber delivery system to guide green light or other appropriate laser wavelength to selectively heat heme proteins in the blood to cause local heating in order to coagulate or cauterize this very small hole to completely close and exit with no damage to the surrounding tissue. This procedure can also be expanded to involve the creation of a very small hole in the cranium (200 micron to 1 mm) to insert the fiber, using the above strategy to enter the interstitial fluid of the brain and allow drugs to be spatially located at the most effective spatial locations for a particular ailment. The ability to spatially target particular locations in the brain allows treatment of pain and reward centres/epicentres for treating chronic pain, addiction, Parkinson and epileptic seizures are representative examples.

This present disclosure allows the injection of controlled doses of drug to the brain by surpassing the blood-brain barrier by creating a pathway for drug delivery small enough to mitigate bleeding and with minimal possible damage to surrounding tissue critical to maintaining brain function. This procedure opens up completely new drug regimes involving drugs of arbitrary mass as well as biological treatments such as antibody/immunotherapy to treat mental illness and optimal recovery of brain function.

The present disclosure solves the problem of overcoming the blood-brain barrier with effectively no damage to surrounding tissue by introducing a hole on the 200 micron scale or smaller that is on the order of the dimension of red blood cells using highly localized PIRL beam delivery through a fiber optic in direct contact to vessel or other tissue to create the aperture on this scale. The surface tension of blood, over such a small aperture, is enough to prevent bleeding while allowing the seating of a needle or hollow fiber to inject drugs past the dense perineurium and endothelial cells lining the blood vasculature of the brain that constitutes the blood-brain barrier. The injection of the drug can be made at specific locations in the brain for optimal delivery and efficacy.

This feature of the present disclosure is made possible by exploiting natural passages such as blood vessels, sinus passages for fiber beam delivery, or by using PIRL based laser disruption and liquification (ablation) to create sub-millimeter access holes through the cranium with no collateral damage beyond a single cell line of the removed tissue. The method of the present disclosure opens up a whole new drug regime, whereby drugs of any mass can be introduced for treating various neurological illnesses such as but not limited to, brain cancer, Alzheimer's disease, Parkinson's disease, chronic pain, mental health disorders such as depression as example applications, and recovery of brain function after neurological injury or illness such as stroke.

According, in another aspect, there is provided a system for penetrating the blood-brain barrier with minimal damage, the system comprising:
a pulsed infrared laser source configured to generate infrared laser pulses;
a laser pulse delivery assembly comprising:
   an optical fiber optically coupled to the pulsed infrared laser source, such that the infrared laser pulses are delivered through the optical fiber to a distal tip of the optical fiber; and
   a cannula configured to receive and mechanically support the optical fiber, such that the distal tip of the optical fiber is extendable to at least a distal end of the cannula for delivering the infrared laser pulses beyond the distal end of the cannula;
a navigation system configured to provide guidance during manipulation of the pulse delivery assembly to position the distal tip of the optical fiber proximal to the blood vessels making up the blood-brain barrier; and
control circuitry operatively coupled to the pulsed infrared laser source, the control circuitry being configured to perform operations comprising:
   controlling the pulsed infrared laser source to emit the infrared laser pulses such that during penetration of the vessels making up the blood-brain barrier by the distal tip of the optical fiber, the infrared laser pulses have laser pulse properties including:
      a wavelength selected such that absorption by a laser-irradiated tissue volume is predominantly due to excitation of vibrational modes of one or more constituents of a laser-irradiated tissue volume of the vessels of the blood-brain barrier;
      a pulse duration that is shorter than a first time duration required for thermal diffusion out of the laser-irradiated tissue volume and shorter than a second time duration required for a thermally driven expansion of the laser-irradiated tissue volume;
      a pulse fluence and the pulse duration resulting in a peak pulse intensity below a threshold for ionization-driven tissue disruption to occur within the laser-irradiated tissue volume; and
      wherein the pulse fluence is sufficiently high to create an aperture in blood vessels of the blood-brain barrier;
   the pulsed infrared laser source thereby being controlled to facilitate local tissue disruption during penetration of the distal tip of the optical fiber into the intracorporeal tissue region, avoiding substantial deformation of the vessel walls of the blood vessels of the blood-brain barrier and facilitating positioning of the distal tip within the blood vessels of the blood-brain barrier.

The control circuitry is configured to control the pulsed infrared laser source to deliver the infrared laser pulses with the laser pulse properties during manipulation of the pulse delivery assembly to position the distal tip of the optical fiber in the brain beyond the blood-brain barrier avoiding substantial tissue deformation and facilitating positioning of the distal tip in the brain beyond the blood-brain barrier.

The optical fiber is extendable beyond the distal end of the cannula to facilitate penetration of the blood-brain barrier by the distal tip of the optical fiber.

The control circuitry is further configured, after extension of the distal tip of the optical fiber into the region of the brain beyond the blood-brain barrier, to deliver a predetermined amount of fluid into the brain.

The system further comprises an additional laser source optically coupled to the optical fiber, the additional laser source being configured to generate laser energy suitable for providing light of a wavelength suitable to selectively excite blood molecules to coagulate blood or cauterize the aperture to prevent bleeding.

The laser pulse delivery assembly further comprises a liquid delivery conduit in flow communication with the distal end of the cannula;
the system further comprising a liquid delivery pump configured to deliver a liquid therapeutic agent to the liquid delivery conduit; and
wherein the control circuitry is operatively coupled to the liquid delivery pump, and wherein the control circuitry is further configured, after extension of the distal end of the cannula into the region of the brain beyond the blood-brain barrier, to perform operations comprising:
   controlling the liquid delivery pump to dispense the liquid therapeutic agent within the intracorporeal tissue region.

The cannula comprises a primary lumen through which the optical fiber is extendable, and wherein the liquid delivery conduit is provided as a side lumen of cannula, the side lumen intersecting the primary lumen at an internal port residing within a distal region of the cannula, such that the liquid therapeutic agent residing in the liquid delivery conduit is brought into flow communication with the primary lumen, for dispensing the liquid therapeutic agent beyond the distal end of the cannula, after retraction of the distal tip of the optical fiber to a location that is proximal relative to the internal port.

The control circuitry is configured to control the liquid delivery pump to deliver the liquid therapeutic agent within the intracorporeal tissue region after having previously delivered thermal therapy to the intracorporeal tissue region.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIG. 1 illustrates the high Interstitial fluid pressure inside a solid tumor, requiring high injection force, and leading to a net outflow of drugs.

FIG. 2A illustrates the deformation of tumor tissue when attempting to penetrate the tumoral tissue with a conventional needle (beveled cannula).

FIG. 2B illustrates the penetration of tumor tissue, in the absence of substantial deformation of the tumor tissue, by an optical fiber that delivers PIRL pulses that produce local disruption and liquification of the tissue.

FIG. 3 plots the threshold fluence, as a function of wavelength, for ionization and for tissue disruption and liquification via the Pulsed InfraRed Laser (PIRL) impulsive heat deposition tissue disruption mechanism. Pulse durations may be controlled, depending on tissue type, to satisfy complete energy confinement to drive tissue disruption for extraction and reduction of interstitial fluid pressure in the targeted tissue. PIRL represents one example approach to achieve these conditions.

FIG. 4 illustrates an example system for PIRL-based local tissue disruption and thermal therapy.

FIG. 5 illustrates the use of an optical fiber to achieve intratumoral access via the delivery of PIRL laser pulses during translation of the optical fiber into the tumor and the subsequent delivery of thermal therapy within the tumor.

FIGS. 6A and 6B schematically illustrate the direct injection of a liquid therapeutic agent into a tumor via initial pulsed infrared laser pulse mediated tissue disruption followed by direct drug injection into the disrupted tissue.

FIG. 7 illustrates an example system for PIRL-based local tissue disruption and drug delivery.

FIGS. 8A, 8B and 8C illustrate an example intracorporeal laser pulse delivery assembly configured for laser-assisted tissue disruption.

FIG. 9A illustrates an example intracorporeal laser pulse delivery assembly configured for laser-assisted tissue disruption and dispensing of a liquid at an intracorporeal target site.

FIG. 9B illustrates an example intracorporeal laser pulse delivery assembly configured for laser-assisted tissue disruption and for performing aspiration biopsy at an intracorporeal target site.

FIGS. 10A and 10B show example views of an intracorporeal laser pulse delivery assembly having a distal elongate portion sufficiently long to reach targets within the body.

FIGS. 11A and 11B shows design schematics for an example embodiment of the distal beam expansion optical element configured to expand the emerging optical beam from the optical fiber to fill a 500 micron diameter distal output aperture that approximately matches the diameter of the outer sheath (not shown) housing the optical fiber.

FIGS. 11C-11G illustrate different example shapes of the distal beam expansion optical element, showing tapered and rounded configurations which can be optimized for laser-assisted tissue piercing with reduced friction and clearance.

FIG. 12A illustrates an example intracorporeal laser pulse delivery assembly configured for laser-assisted tissue disruption and incorporating a thin elstomeric sheath forming a passive distal valve for dispensing of a liquid at an intracorporeal target site.

FIG. 12B illustrates an example intracorporeal laser pulse delivery assembly configured for laser-assisted tissue disruption and having an intergrated plunger for dispensing of a liquid at an intracorporeal target site.

FIG. 13 illustrates an example intracorporeal laser pulse delivery system configured for laser-assisted tissue disruption and using a multi component, and multi step process, which includes, as a first step, laser enabled piercing to the target region, followed by laser induced disruption of a volume of tissue at the target, aspiration of the laser, liquified tissue, and finally injection of the volume of drug into the disrupted volume at the target.

FIG. 14A illustrates an example system for PIRL-based local tissue disruption, drug delivery, and photodynamic activation.

FIGS. 14B, 14C, 14D, 14E, 14F and 14G illustrate the use of laser assisted injection of photodynamic drugs, where the drug is designed for faster uptake than diffusion out of the tumor boundaries and the drug is activated by an additional light source resonant with photolabile groups attached to the drug molecules to create a photoadhesive which will be bound in position while creating singlet oxygen for destruction of cancer cells.

FIG. 15 illustrates the use of the optical fiber for photo fixing of drugs after local drug delivery.

FIG. 16 illustrates the use of the optical fiber to deliver additional PIRL pulses after local drug delivery to perform photomechanical-enhanced drug diffusion.

FIG. 17 illustrates the use of the optical fiber to perform photothermal treatment of the tissue region.

FIG. 18 illustrates extended-duration photodynamic therapy via an optical fiber that remains inserted in the patient after initial treatment.

FIG. 19 illustrates the use of an optical pressure sensor for the detection of tumoral interstitial fluid pressure (IFP).

FIG. 20 is a CAD drawing showing an example implementation of the embodiment shown in FIG. 12.

FIG. 21A shows a photograph of an example implementation of the embodiment shown in FIG. 20, with a modification to include a distal removable portion that includes a fiber connector at the input side of the fiber optic for attachment to a fiber optic extension (patch cable) for delivery of laser energy to the distal section.

FIG. 21B shows a prototype embodiment of the injector design of FIG. 12B created by modification of a standard syringe pump.

FIG. 21C shows another prototype embodiment of the injector design of FIG. 12B in which a motor is incorporated into the rigid housing.

FIG. 22 shows a schematic representation of embodiment which includes motorized motion for the piercing action and force sensor for monitoring of the frictional forces for optimal piercing speed and laser parameters.

FIG. 23 illustrates an example system for performing PIRL-based local tumor disruption and direct drug injection using a laser pulse delivery tool having a distal optical waveguide for beam delivery and an integrated fluidic channel for targeted drug delivery.

FIG. 24A shows an example stereotaxic guided system for direct, transcranial access to the cerebral arteries through the skull.

FIG. 24B shows a laser example of a microcannula that is guided directly into a cerebral artery via a first puncture and then into the target interstitial region of the brain via a second puncture, effectively travelling through the vessel perpendicular to the direction of the vessel.

FIG. 25 illustrates the sequence of events when a needle is penetrating an anatomical vessel as currently practiced. The tissue both deforms and deflects with the adhesion of the needle and forces applied

FIG. 26 illustrates the sequence of events for penetrating the blood-brain barrier using a first embodiment of a device designed for this procedure. The tissue is locally disrupted, liquified like a knife in hot butter, by the action of the PIRL beam delivered through a tapered fiber tip, to allow the creation of an aperture too small for blood leakage and with no forces leading to deflection or deformation of tissue. The tissue stays within elastic limit and seals upon exit like a septum. The energy is fully confined to the 10 micron IR absorption depth of water in the tissue in the longitudinal direction, scale of single cells, and the diameter of the fiber in the lateral direction which can be on the order of 100 microns or dimensions of fewer than 10 cells in diameter.

FIG. 27 is similar to FIG. 26, illustrating the sequence of events for penetrating the blood-brain barrier specifically in which the aperture created lead to blood leakage. The sequence shows the use of green laser light from the same fiber as the PIRL beam to enable selective excitation of blood to locally heat the blood in the exact location of the aperture to coagulate and seal or to cauterize the tissue as needed to stop the bleeding.

FIG. 28 is essentially a blowup of panel (d) in FIG. 27 which illustrates using a green light beam upon withdrawal of the needle selectively excite blood thereby inducing coagulation and/or cauterization to stop the bleeding.

FIG. 29 shows the cannula or needle injecting drug towards thermally expanding blood/liquid flow and bubble stream, (solid circles) which keeps the aperture past the blood-brain barrier open.

FIG. 30 illustrates an example system for performing PIRL-based local tumor disruption and direct drug injection using a laser pulse delivery tool having a distal optical waveguide for beam delivery and an integrated fluidic channel for targeted drug delivery.

FIG. 31 shows an example PIRL laser or other system with sufficiently short IR laser pulses for delivery and fluid delivery assembly that includes a hollow optical fiber and a distal sapphire tip with lateral irrigation channels.

FIG. 32 shows portion of the present system for penetrating the blood-brain barrier used to delivery fluids once the blood-brain barrier has been crossed.

FIG. 33A shows schematic illustrations of the vascular anatomy of the brain.

FIG. 33B is a table showing the diameter and minimum bend radius of several cerebral arteries.

FIG. 34 schematically illustrates a microcatheter system for intracranial delivery of a pharmaceutical.

FIG. 35A illustrates an example of a microcatheter system that includes a delivery catheter for navigating to the cranial vasculature and an extendable microcatheter that includes an optical fiber and a fluid delivery channel.

FIG. 35B illustrates an example of a microcatheter system that includes a delivery catheter for navigating to the cranial vasculature and an extendable, steerable microcatheter that includes an optical fiber and a fluid delivery channel.

FIG. 35C illustrates an example of another example of a microcatheter system that includes a delivery catheter for navigating to the cranial vasculature and an extendable, steerable microcatheter that includes an optical fiber and a fluid delivery channel which is steered by deforming the distal tip using tendons connected to a proximal tendon tension controlling mechanism.

FIGS. 36A, 36B, 36C, 36D and 36E illustrate an example method in which the distal end of the microcatheter is positioned adjacent to a vessel bend and extended through the vessel wall while delivering PIRL laser pulses to facilitate disruption of the vessel wall, followed by dispensing of a pharmaceutical fluid beyond the vessel wall.

FIGS. 37A, 37B, 37C, 37D and 37E illustrate an example method in which the distal end of the microcatheter is steered adjacent to a vessel wall and extended through the vessel wall while delivering PIRL laser pulses to facilitate disruption of the vessel wall, followed by dispensing of a pharmaceutical fluid beyond the vessel wall.

FIGS. 38A and 38B illustrate an example method in which the distal end of the microcatheter is adjacent to a vessel wall and PIRL laser pulses are delivered to facilitate disruption of the vessel wall, followed by dispensing of a pharmaceutical fluid proximal to the disrupted vessel wall, such that the pharmaceutical fluid extends through the disrupted vessel wall.

FIG. 39 illustrates a method of vascular wall coagulation via the delivery of additional non-PIRL laser pulses.

FIG. 40 shows an example method in which a microcatheter, delivered to a cranial vessel via a microcannula (not shown), is subsequently delivered to an intravascular location for PIRL-assisted delivery of a pharmaceutical fluid.

FIG. 41 Illustrates an alternative delivery mechanism for a laser-based system employing impulsive heat deposition to disrupt tissue, wherein a thin layer is deposited on the distal tip of a fiber optic. In this novel approach, a nanosecond pulsed laser, operating in the mid-IR, near-IR, or visible spectrum, is used to irradiate the fiber optic tip coated with a mechanically strong, highly absorptive material.

FIG. 42 shows ultrasound images demonstrating the ability to localize to tip of an optical fiber emitting PIRL pulses via the detection of photoacoustic signals.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. Unless otherwise specified, the terms “about” and “approximately” mean plus or minus 25 percent or less.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein. Unless otherwise specified, the present disclosure relates to and explicitly incorporates each and every specific member and combination of sub-ranges or sub-groups.

As used herein, the term “on the order of”, when used in conjunction with a quantity or parameter, refers to a range spanning approximately one tenth to ten times the stated quantity or parameter.

As used herein, the phrase “microcannula” refers to an elongate conduit having a diameter less than 1 mm. A distal end region of a microcannula may have a variety of shapes, such as, for example, a sharp, needle-like (pointed) distal shape, a blunt distal shape (e.g. a truncated cylinder), and a beveled shape.

As described above, cancer drugs are typically designed for systemic or full body exposure to kill the primary cancer and any migratory cells that might lead to metastases of the cancer. This puts enormous constraints on drug design to be highly specific for rapidly dividing cancer cells in which the cancer mass can be >10⁴ smaller than rest of the body mass. This high contrast is needed to try to selectively kill cancer cells over healthy cells, however, the required contrast is not perfect, which often leads to the acute and debilitating side effects associated with chemotherapy. New classes of drugs can be designed for rapid uptake rather than high selectivity to a given cancer profile. The action volume of the drug could be determined by the diffusivity of the drug into a given tissue.

It would therefore be desirable to be able to directly and locally inject a drug to tumor to provide targeted therapy and avoid the complications and limitations of conventional chemotherapy.

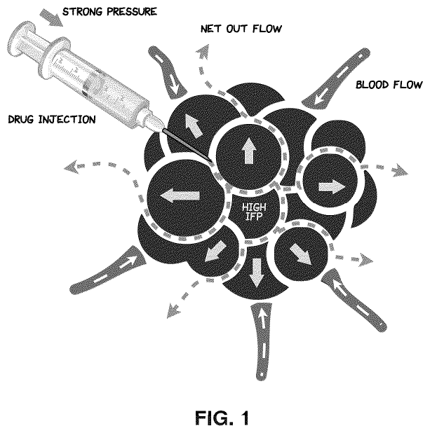

Given a well-defined cancer location, the success of direct injection with the use of a needle of a cancer drug at the site of the tumor is limited by the high osmotic pressures associated with cancerous tissue, which leads to a net outflow of the drug rather than enabling site selective treatment of just the cancer tissue, as shown in FIG. 1. In addition, the uptake of the drug is further complicated by out-diffusion away from the cancer site. There is no conventional means to arrest the diffusive loss of drug at the targeted site. The pressure gradient and associated net diffusion process leads to a loss in local concentration of the drug at the cancer site. There is a competition between drug uptake (hours) and diffusion away (seconds to minutes) from the cancer site. It is these two processes that lead to loss of efficacy in uptake of the drug in using any conventional means to deliver cancer drugs at an injection site. The same problem exists for treatment of most intracavity, localized, diseases.

The problem is exacerbated by the occurrence of chemo-immunity where, even a powerful drug that works selectively for a given type of cancer, can further influence the evolution of the cancer tissue morphology and lead to increased osmotic pressure and abnormal vasculature that can further exasperate the problem, which leads to a form of immunity from the drug action, as mentioned above. Accordingly, the direct delivery of drugs by use of needles has failed as a consequence of the enormous interstitial fluid pressure (IFP) that prevents physical delivery of the required dose to the cancer site.

These effects limit the drug dose from reaching the critical concentration required (LD50 for cancer cells) to perform the intended function. If a drug were provided in a uniform distribution, the drug would be selectively taken up by the targeted cancer site. However, the spatial gradient in diffusion at the cancer site blocks this condition. This effect occurs frequently and arises from the high degree of vascular abnormality and buildup a high osmotic pressure with further changes in tissue morphology induced by the drug action itself.

Indeed, in some solid tumors the osmotic pressure becomes so high that it is physically not possible to inject the drug with normal needle stock and pressure delivery. It is simply a matter that it is not possible to get the drug to the cancer location due to this enormous pressure gradient inherent to cancerous tissues. As noted above, the subsequent problem is normal diffusive dispersion of the drug from the site even when site-selective delivery is possible. The present disclosure provides solutions to this diffusion problem in both the regimes of normal and cancerous tissue.

Another problem with direct injection of drugs into solid tumors is access to the tumor location. Large open wound surgeries cause significant trauma and are a limiting factor in the number and frequency of treatment of solid tumors in multiple different locations. The use of surgical intervention to remove cancerous tissue is limited in scope due to the significant trauma to surrounding tissue and loss of function. Usually only one or few surgical procedures can be tried to eradicate cancer. Metastasis to multiple sites leads to stage 4 cancers that no longer can be removed surgically to extend life. Less invasive procedures can be attempted to minimize the damaged volume of tissue such as needle aspiration or related delivery methods.

However, a hollow needle mechanically pierced into the body can cause trauma through the shear force needed to push the needle to the targeted tissues site, limiting the ability repeat treatment. In addition, numerous flexible guided needles have been developed to reach targets inside the body via curved entrance paths which also lead to damage to tissue along the entrance wound (e.g., Van de Berg, Nick J.; van Gerwen, Dennis J.; Dankelman, Jenny; van den Dobbelsteen, John J. (2014). Design Choices in Needle Steering&#x2014; A Review. IEEE/ASME Transactions on Mechatronics, ( ), 1-12.doi: 10.1109/TMECH.2014.2365999).

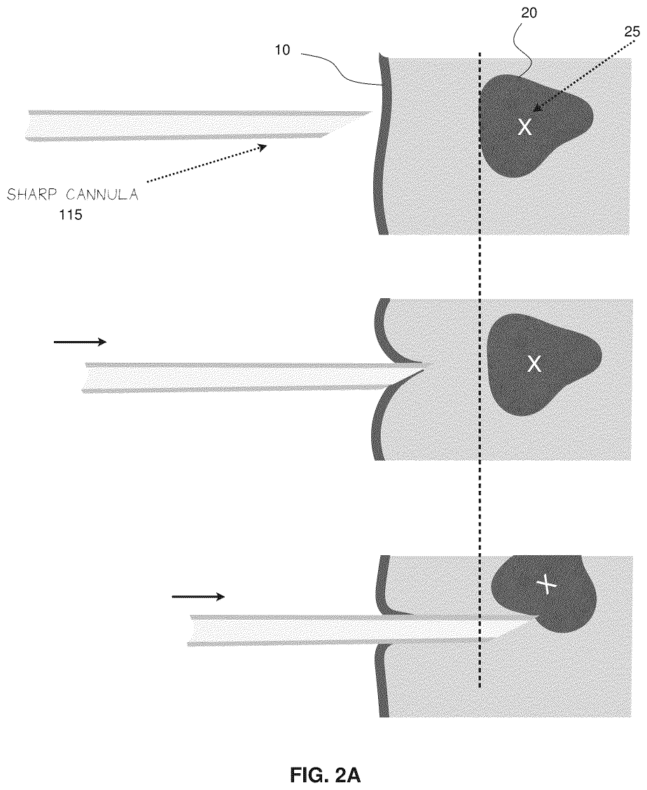

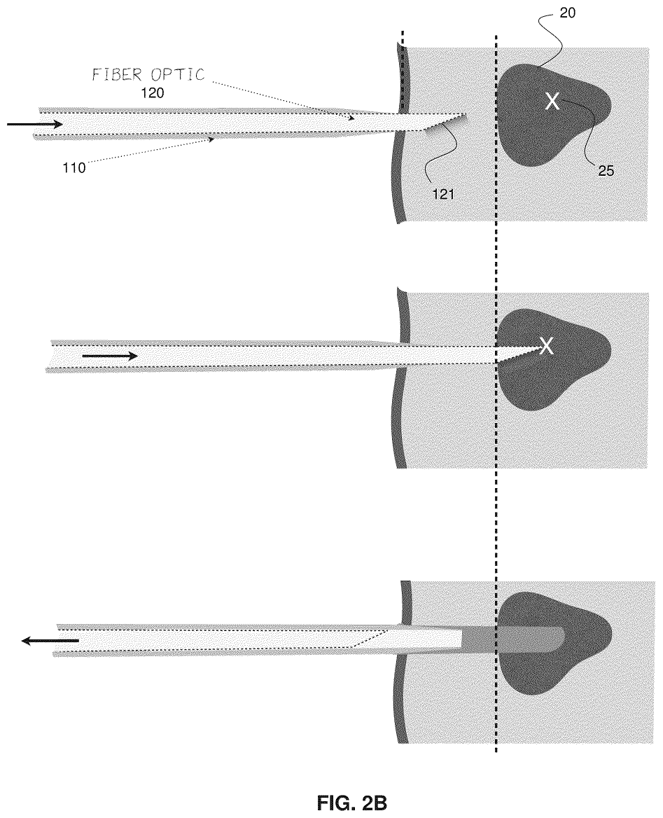

FIG. 2A illustrates the challenges faced when attempting to penetrate tumor tissue 20 and reach a target location 25 using a conventional needle 115 having beveled tip (e.g. a needle). As the needle 115 is advanced to initially contact the skin surface 10, the surface deforms, and as the needle breaks through the tissue surface, the surface is locally depressed (dimpled). Eventually the elastic limit is reached and the tissue spreads to accommodate the needle. As the needle continues to advance into the tissue, the surrounding tissue deforms further. The cells are displaced or pushed apart as the needle advances towards the tumor and cuts a path through the tissue. The needle compresses and deforms tissue, enroute to the desired location within the tumor. The needle causes damage to tissue by the both the shear force trauma of the needle entry and the deformation of tissue, which causes difficulty in reaching the desired region, especially in the presence of significant tissue heterogeneity.

When the needle contacts the tumor, it does so at a location that is offset from a planned location due to deformation of the tumor caused by advancement of the needle and deformation of the tissue surrounding the tumor. Further advancement of the needle causes further deformation of the tumor, as the high interstitial fluid pressure of the tumor resists penetration of the tumor by the needle, and the distal tip of the needle 115 fails to reach the target location 25 within the tumor 20.

The present inventors, when setting out to solve the aforementioned problems associated with local delivery of a therapy to a tumor site, identified the following challenges: 1) the need to guide an optical fiber to tumor tissue without inducing substantial tissue deformation, to enable more accurate targeting of the tumor, 2) the ability to penetrate a tumor and overcome the interstitial fluid pressure to facilitate the delivery of the necessary volume of the drug, delivered either in a solution or gas; and 3) the need to limit diffusion of the drug away from the cancer site once delivered, in order to enable drug uptake. The need to block diffusion away from the cancer was deemed by the inventors to require a direct intervention to change the flow gradients responsible for the high interstitial fluid pressure in cancerous tissue. The present inventors therefore sought a solution that would facilitate the local delivery of therapy targeted at an intracorporeal tissue region (e.g. a tumor or other region of tissue pathology), preferably while only substantially affecting the intracorporeal tissue region, without causing significant trauma or deformation to neighbouring tissue, thereby potentially enabling the procedure to be repeated multiple times without compromising quality of life.

As explained in detail below, the first and second challenges may be overcome with the use of a pulsed (e.g. ps or ns) infrared laser that delivers pulses having properties that result in local disruption and liquification of the tissue, enabling penetration of tumor tissue and the creation of pathways inside the tumor that are much smaller than would be possible with mechanical entry injection needle, and without substantial deformation of tissue as typically associated with needle-based biopsies.

This approach provides access to the tumor site with substantially less trauma than conventional approaches and enables selective control of energy deposition solely at the tumor site, which may be beneficial in reducing the elevated pressure of the tumor and facilitating the subsequent local dispensing of a therapeutic agent within the tumor.

Accordingly, various example embodiments of the present disclosure provide systems and methods that advantageously employ optical-based local tissue disruption and liquification to facilitate the direct delivery of local therapy to an intracorporeal tissue region (a tissue region within the body). As will be described in detail below, local tissue disruption and liquification can be achieved using a pulsed infrared laser that is configured to deliver pulses that selectively target vibrational absorption in the tissue and are delivered with a suitable pulse duration and fluence. For example, the wavelength of the infrared laser pulses can be selected to target vibrational absorption of water to create highly localized tissue disruption due to the extremely strong absorption of infrared in the OH-stretching region, with absorption 1/e depths on the order of 1-10 microns, which is smaller than a single cell dimension.

A pulsed infrared laser system that is configured for the delivery of laser pulses having pulse conditions suitable for performing tissue disruption according to the aforementioned mechanism and the conditions described in further detail below is henceforth referred to as a “PIRL” (pulsed infrared laser) system. Likewise, infrared laser pulses having wavelengths, pulse durations and energies suitable for performing tissue disruption and liquification according to the aforementioned mechanism are henceforth referred to as a “PIRL” pulses. It will be understood that a PIRL pulse is not limited to a picosecond pulse, as preferred pulse durations for some wavelengths extend into the tens of nanosecond range, as described below.

PIRL laser pulses are infrared laser pulses that are sufficiently short to drive tissue disruption and liquification faster than the timescales associated with thermal and acoustic transport, thus avoiding damage due to heat and shock wave formation, while also being sufficiently long to avoid the ionizing radiation effects of plasma formation. PIRL pulses are provided with a wavelength selected such that absorption of the laser pulses by tissue is predominantly due to excitation of vibrational modes of one or more constituents of the tissue, such as water. Example suitable wavelength ranges for PIRL laser pulses therefore include 2.7-3.3 μm, 5.9-6.1 μm and 1.8-2.0 μm. Future developments in high energy and short pulsed laser sources will enable PIRL tissue disruption and liquification by targeting vibrational absorption in target molecules between 2-20 μm.

For example, the PIRL laser pulse wavelength may be selected to overlap with, or reside proximal to, a strong peak in the vibrational spectrum of a constituent of the tissue, such as the CC-stretch region of collagen or N—H stretch of amino acids in proteins, where there is less water for energizing materials. Such vibrational modes quickly absorb the electromagnetic radiation and may effectively localize optical energy to micron scale deep sections of the exposed tissue. In the case of water, maximum absorption for vibrational modes occurs between about 2.7-3.33 μm, where broad peaks, >10 cm⁻¹, in the absorption spectrum, correspond to the short lived, subpicosecond to picosecond, relaxation of the OH-stretching vibrational modes of liquid water molecules to energize the surroundings. The spectrum also shows the resonance conditions between the OH-stretch and other vibrational modes such as the OH bend and intermolecular modes. Other absorption peaks, for example, at approximately 1.9 μm or approximately 6 μm, may alternatively be employed, as described in further detail below.

In various example embodiments, PIRL pulses are generated and delivered such that when a given volume of tissue is irradiated, the pulse duration is shorter than (i) the time duration required for thermal diffusion out of the laser-irradiated volume of tissue, and (ii) the time duration required for a thermally driven expansion of the laser-irradiated volume of tissue. The skilled artisan will be able to determine a suitable pulse duration for PIRL pulses for a given pulse wavelength and absorption depth in tissue (e.g., in a given type of tissue). In general, for a given PIRL laser pulse wavelength that is selected according to the aforementioned criterion (absorption of the laser pulses by tissue is predominantly due to excitation of vibrational modes of one or more constituents of the tissue), the known properties of the tissue, such as the absorption depth of the laser pulses, thermal diffusion constant, and the speed of sound, may be employed to calculate a suitable PIRL pulse duration that satisfies criteria (i) and (ii) above. Alternatively, or additionally, experiments may be performed to determine a suitable laser pulse duration that satisfies criteria (i) and (ii).

For example, in the case of disruption and liquifying tissue with a laser wavelength of 3 μm, for which the absorption depth is approximately 1 μm, the maximum pulse duration can be calculated based on the ratio of absorption depth to speed of sound, 1730 m/see, i.e. t=a/v=10-6 m/1.730×103 m/s=5.78×10⁻¹⁰ sec, giving approximately 600 ps (e.g. see Duck, F. A., Physical Properties of Tissue, Academic Press, London, 1990, and Duck, F. A., Propagation of Sound Through Tissue, in “The Safe Use of Ultrasound in Medical Diagnosis”, ter Haar G and Duck, F. A, Eds., British Institute of Radiology, London, 2000, pp. 4-15).

Different tissue types (e.g., bone, brain and skin) will have different absorption depths at a given wavelength. Around the OH-stretching band, the absorption of the tissue is dominated by the water content. In general, the absorption depth will be longer than pure water. At a wavelength of 2.95 μm, the absorption depth of pure water is close to 0.7 μm, and given the variance in the high concentration of water in different tissues, along with other OH-stretching modes in the tissue, the absorption depth may be approximately 1-2 μm at this wavelength. If the wavelength of the laser is shifted, e.g., to a wavelength of 2.75 μm, then the absorption depth of the light increases by a factor of about 3 according to the change in the absorption spectrum of the OH-stretch. (See, for example, Diaci, J., J. Laser and Health Acad. 2012, 1-13 (2012).

In another example in which tissue is disrupted and liquified using a laser wavelength of 6 μm, for which the absorption depth is approximately 100 μm, the pulse duration should be chosen as shorter than 100 μm/1.753×103=57 ns. Likewise, an absorption depth of 100 μm is expected to occur for a laser wavelength of 1940 nm. Accordingly, a suitable pulse duration for PIRL pulses will depend on the pulse wavelength. In some example implementations, a suitable pulse duration for PIRL pulses may range from 100 ps to 100 ns, depending on the selected wavelength and light intensity dependent changes in absorption depths due to saturation of the absorption at a given wavelength.

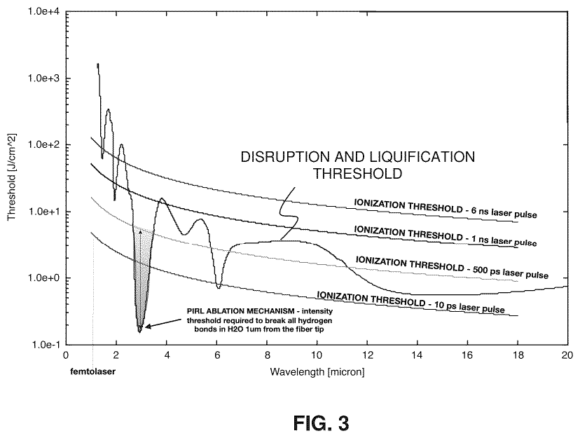

The pulse duration and pulse fluence are also selected such that a peak pulse intensity is below a threshold for ionization-driven ablation to occur within the laser-irradiated volume of tissue. For example, for a given pulse duration, a suitable upper limit of the pulse fluence may be determined to avoid the threshold for ionization-driven ablation. In the example case of human skin tissue, at a laser wavelength 3 μm, the maximum fluence values for avoiding ionization-driven ablation, for pulse durations of 10 ps, 500 ps, and 1 ns, are approximately 1.5 J/cm², 5.5 J/cm², and 17 J/cm², respectively, as shown in the FIG. 3.

Furthermore, in order to achieve PIRL-based disruption and liquification of tissue for laser pulses that satisfy the preceding criteria involving wavelength, pulse duration and pulse fluence, the laser pulses should be provided with a sufficient pulse fluence to achieve a threshold energy density for PIRL tissue disruption and liquification, as shown, for example, by the tissue disruption and liquification threshold identified in FIG. 3. For example, the pulse fluence that is delivered to the tissue should be sufficiently high so that the energy deposited in the irradiated volume is sufficient to heat the contents of the volume up to its vaporization temperature including the enthalpy of vaporization.

For example, if the beam is focused to 200 μm (or a 200 μm core diameter fiber is used in contact) and ablates a volume of ˜1 μm deep×π(100 μm) 2, the mass of the ablated volume is 3.1×10⁻⁸ g in the case of water and 3.4×10⁻⁸ g in the case of skin (which has a density of 1.15 g/cm³). The energy required to raise the temperature of this volume of water from 20 to 100° C. and then vaporize the volume is approximately 80 μJ of energy, which corresponds to a fluence of 0.25 J/cm² for a 200 μm spot. This fluence defines the threshold for impulsive heat deposition to drive the phase transition without loss due to acoustic transport or thermal diffusion out of the excited zone. To ensure the ensuing tissue disruption and liquification process occurs in this limit, for highly scattering medium such as tissue which effectively decreases the incident intensity, typical excitation conditions used are 1 J/cm². The determination of a sufficient fluence for PIRL tissue disruption and liquification can be made experimentally by varying the applied fluence, examining the resulting tissue disruption and liquification, and selecting an applied fluence value that provides a sufficient amount or degree of tissue disruption and liquification. The subsequent process in the confined volume, defined by the unexcited tissue and the fiber tip, leads to disruption of the tissue to cellular levels and removal of pressure gradients.

In the present implementation of contact mode delivery of PIRL pulses, localized tissue disruption/liquification occurs and allows the optical fiber to advance without substantial shear friction (analogous to a hot knife in butter), leading to fine tissue disruption, tissue homogenization, and removal of vasculature and interstitial fluid channels. In the present direct-contact implementations, the optical fiber closes the space to confine the energy. Unlike ablative PIRL that employs a non-contact mode to facilitate ablative vaporization, contact-mode PIRL does not result in transduction of the energy into translational motion as an ablation plume with material removal, but rather leads to homogeneous nucleation involved in phase transitions whereby the energy surpasses the barrier to liquify solid material and form gas bubbles from lowest vapour pressure constituents. Without intending to be limited by theory, it is believed by the inventors that the nucleation process and bubble formation occurs in a spatially uniform manner, corresponding to the energy distribution deposited in the tissue by the fast conversion of absorbed infrared radiation into thermal motions. These nucleation sites merge at longer times (>10-100 ns) and create shock waves, and this process is highly localized through the initial uniform homogeneous nucleation that occurs with the PIRL process. Ultrasound imaging has shown these uniformly generated nucleation sites to be very effective in deliberately disrupting tissue with a very fine dispersion of tissue. This process effectively liquefies tissue. Moreover, this process is cumulative locally. One can deliberately control the number of pulses to drive additional energy into the exposed volume to achieve thermal disruption which disperses to increase the volume of tissue disruption. Accordingly, in some example implementations, trains of pulses of varying lengths can be employed to increase the volume of tissue disrupted in a controlled manner to extend the tissue disruption beyond the single pulse limit of a few 10 s of microns and to control the degree of tissue heating as desired.

When PIRL pulses are delivered to a local tissue region within the body, through an optical fiber inserted into the body, the resulting local tissue disruption and tissue liquification enables atraumatic and accurate guidance of the distal tip of the optical fiber to an intracorporeal tissue region of interest, and facilitates direct entry of the distal tip of the optical fiber into a selected intracorporeal tissue region, such as a tumor or other region associated with pathology.

These beneficial properties of an optical fiber delivering PIRL pulses is illustrated in FIG. 2B, in which a microcannula 110 is shown housing an optical fiber 120 that is interfaced with a pulsed infrared laser source and delivers PIRL pulses from its distal tip 121. The PIRL pulses disrupt the local cellular structure and cause local tissue disruption and liquification in the laser irradiated tissue volume that lies beyond the distal tip of the optical fiber, enabling the advancement of the optical fiber along a substantially straight path, without applying a deformation-inducing force to the surrounding tissue.

As shown in the figure, the initial penetration of the tissue is facilitated by the delivery of PIRL pulses by the optical fiber, which causes local tissue disruption and liquification as the distal tip passes directing into the tissue, without substantial resistance, and without dimpling and deformation of the tissue surface. Accordingly, the fiber punctures the tissue surface with significantly less force and friction than in the conventional approach shown in FIG. 2A. The disrupted cells in the form of a liquid squeeze out behind the distal portion of the optical fiber. While the figure illustrates an example case in which the optical fiber is extended from the distal end of the microcannula during PIRL-mediated penetration, insertion and translation into the tissue, in an alternative implementation, the distal tip of the optical fiber may reside adjacent to the distal end of the microcannula 110 during PIRL-mediated penetration, insertion and translation, with the PIRL pulses causing tissue liquification in the direct path of the distal end of the microcannula.

As shown in FIG. 2B, at least a proximal portion of the optical fiber may be housed within a microcannula to mechanically support the optical fiber as it is guided within the body. When a microcannula is employed during guidance of the optical fiber within the body, the microcannula preferably has an outer diameter, at least within a distal region of the microcannula, that only exceeds that diameter of the optical fiber by a small margin, such as less than 5%, 10%, 15%, 20%, or 25% of the diameter of the optical fiber, in order to ensure that the additional thickness of the microcannula does not result in substantial tissue trauma as the microcannula is advanced in the body. In some example embodiments, the diameter of the microcannula tapers down in a distal direction toward the distal aperture to reduce friction and allowing the laser to ‘clear’ the path, while the microcannula stretches its way into the tissue with minimal drag.

As shown in FIG. 2B, the tissue disruption and imparted fluid flow properties allows the fiber to advance to the tumor with little or no discernable friction or shear sticking force. This property enables clear trajectory paths to the tumor site without substantial tissue deformation and ensures arriving at the desired targeted tissue or region of interest. As will be described in further detail below, this property of fiber-delivered infrared laser pulses also enables the injection of therapeutic agents without substantial damage to the tissue along the entry pathway to the target site and ensures arrival at the target site without substantial tissue deformation.

FIG. 2B also shows how the disruption and liquification of tissue by the PIRL pulses emitted by the distal tip of the optical fiber enables the distal tip of the optical fiber to enter the tumor 20 without incurring a significant resistance and without being significantly deflected. As a consequence, the PIRL pulses emitted by the optical fiber facilitate the direct and unimpeded entry of the optical fiber into the tumor 20, in the absence of substantial deformation of the tumor, enabling the positioning of the distal tip of the optical fiber at the desired intratumoral location 25.

The PIRL pulses delivered within the tumor reduce the barriers to free flow of interstitial fluid into hypoxic portions of the solid portion of the tumor. The local disruption of the tumor tissue that is caused by the infrared laser pulses is similar in concept to the local high-energy radiation therapy (e.g., the “gamma knife” or “cyberknife” technologies) that employ concentrated gamma or high-energy x-ray radiation to create local spurs of ionization at select sites. Such systems require major instrumentation/cost in the form of a particle accelerator to obtain the desired radiation, and such methods operate on a probabilistic model for spur formation. The systems and methods of present disclosure, in contrast, can be implemented using a compact, comparatively low-cost, table-top laser with an optical fiber for delivery of the laser energy in a form that effectively creates its own “tunnel” to the specific target site, where the target site is known from preoperative imaging.

Some example embodiments of the present disclosure employ a thin rigid or flexible microcannula to facilitate insertion and guidance of the optical fiber into the body. In other example embodiments, a fiber may be employed, in the absence of a supporting microcannula, to access and provide local therapy to an intracorporeal tissue region, with atraumatic fiber delivery, and accurate fiber positioning, facilitated by the emission of PIRL pulses and the resulting disruption and liquification of tissue residing beyond the distal tip of the optical fiber. Indeed, in cases in which the optical fiber is guided in the absence of microcannula, the wound size is only slightly larger than the fiber diameter, which may be, for example, in the range of 50-200 microns in diameter. Due to the small size of the disruption filament and the non-thermal disruption mechanism, minimal damage is caused to the tissue along the path towards the final target within the body.

In some example implementations, an optical fiber may be inserted, without mechanical support by a microcannula (e.g., extended from a microcannula, or inserted without a cannula), approximately 5 cm into the body without substantial risk of breakage for soft tissue. An optical fiber may be extended up to this depth by control of the advancement of the optical fiber for example, at a low laser repetition rate, such as 10-100 Hz, to allow the optical fiber to advance without thermal accumulation to cause damage. It is expected that more rigid tissue structures will deform around the optical fiber, but if the fiber movement is too fast there will be resistance that may break the fiber. With 5 cm penetration, most positions in the body can be accessed. It is expected that this depth can be increased to 10 cm or more, providing full access within the body, using a microcannula or line-of-sight laser drilling deeper into the tissue with aspiration of the disrupted/liquified tissue.

For structures that are more rigid, such as collagen or ligaments that are in the way of the path to the tissue of interest, another laser could be used to create a small access hole (with the laser energy from the other laser being coupled into and delivered through the same optical fiber). Examples of suitable lasers include an excimer or 266 nm Nd based laser, which can be employed to create the pathway. In such a case, it may be beneficial to include a means of removing the disrupted tissue to reduce the buildup of retarding force and chance of inhomogeneous stress leading to fiber breakage.

For example implementations that employ a microcannula for fiber support, the microcannula (or other support structure) could be positioned to a depth that is close to the surface of the body, or into the body to a location below the surface where damage is minimal. The optical fiber can then be carefully advanced, withdrawn, material aspirated to reduce back retarding force, and the optical fiber may then be advanced further beyond cleaned out point. This procedure could be performed intermittently during advancement of the optical fiber, for example, every 5 mm of advancement (it will be understood that the distance between aspiration events will be tissue specific). In many cases, it is expected that the local disruption and liquification of tissue by the PIRL pulses will provide sufficient liquification of the tissue to permit the liquified tissue to pass longitudinally along the fiber or microcannula outer surface as the fiber is advanced into the body.

In some example implementations, one or more optical fibers (e.g., an optical fiber bundle) may be employed to facilitate an intracorporeal (e.g. endoscopic) pulsed infrared laser beam delivery device having distal transverse cross-sectional dimensions less than one millimeter, unlike previous intracorporeal (endoscopic) approaches which necessarily involve dimensions well exceeding 1 mm. For example, in some example implementations, as described in further detail below, the distal region of the microcannula may have a diameter less than 300 microns or even less than 200 microns for beam delivery for minimal damage to the tissue along the entry pathway. The use of an optical fiber is beneficial for the delivery of infrared laser pulses both for creating the minimally invasive path to the target tissue and for eradication of the targeted tissue.

Various embodiments of the present disclosure may thus be employed to solve the problem of cancer removal without causing substantial trauma, thereby enabling removal of cancer that can be detected within the body, even in cases of where the cancer has metastasized. Conventional treatment of cancer often stops at stage 4 where metastases has occurred as surgical intervention is no longer an option due to excessive risk in current invasive surgical procedures. The embodiments of the present disclosure provide a novel means of energy delivery without any shear damage to surrounding tissue to gain access to region of interest. Multiple surgical procedures can be executed to remove multiple cancers without trauma even for stage 4 patients and for greater efficacy of drug delivery.

With fiber delivery of PIRL pules, the entry level wound can be on the order of 10-20 cells in diameter and without shear induced damage, elastic rebounding of tissue occurs to close the wound, which can be affected without substantial damage along the entry path to the targeted tissues to be removed. In contrast, in the case of a conventional needle, there are shear forces in the insertion process that lead to inflammation around the entry path and it is this effect that is responsible for some of the action and benefits attributed to acupuncture. Further, the very action of the shear forces involved and differential stickiness, surface adhesion, or variations in tissue stiffness leads to deflections of needles from the target site making it difficult to place the needle on target and often requires multiple attempts to statistically improve the chance of docking the needle at the desired site.

Some example embodiments of the present disclosure facilitate the insertion of an optical-fiber-based microcannula having sub-millimeter distal cross-sectional dimensions, significantly smaller in diameter than acupuncture needles. By the action of the PIRL guided in the optical fiber and the resulting tissue interaction, the act of optical fiber entry creates its own path, without significant collateral damage or excessive shear force, to go directly to the desired site without tissued deformation or deflection of path, leading to wound-free entry into any part of the body along a preferred or surgically essential pathway. The process can be likened to a “hot knife going through butter” but with minimal (in some cases, effectively zero) restructuring of local tissue (butter in this analogy) or changes due to shear forces. The resulting wound heals without scar tissue formation to give the absolute minimum trauma to the body to allow removal of diseased tissue. This feature enables multiple surgeries without risk or undo trauma to the patient for all procedures with especially important applications for removing solid tumors for patients to give an extended and higher quality of life.

Current approaches to minimally invasive surgical interventions, such as manual/robotic endoscopic or laparoscopic surgery, can be performed using surgical navigation (guidance) methods, but the cross-sectional dimensions of such devices are still relatively large, with trocar sizes typically in the range of 8.5 mm to 12 mm. These methods of intrusion necessarily introduce damage, especially in entering soft tissue, where shear forces lead to local damage and inflammation, even If healing of the entry wound is not debilitating.

The path of the fiber to this site can be tracked in real time with minimal tissue deformation during passage to ensure absolute targeting of the desired tissue, or precise location. In some example implementations, an assembly that includes an optical fiber residing with, an optionally extendable from, a microcannula (or in some cases, an optical fiber absent of a supporting microcannula), can be guided and adjusted in real time with one or more imaging methods such as, but not limited to, ultrasound imaging, magnetic resonance imaging, fluoroscopy, computed tomography, angioscopy and electromagnetic position sensing, optionally employing one or more detectable markers residing on the microcannula and/or optical fiber, and optionally also providing surgical guidance based on intraoperative volumetric image data that is rendered in an intraoperative frame of reference and presented on a user interface.

In some example embodiments, the optical fiber tip may be detected and localized by ultrasound imaging of a photoacoustic signal generated by the local disruption of tissue by the PIRL pulses. For example, ultrasound imaging may be employed to detect shock waves that enable localization of the distal tip of the optical fiber. Such an embodiment may be beneficial in providing an improvement of localization accuracy over what is achievable based on the detection of ultrasound echoes from the tip region of a sapphire optical fiber, as ultrasound artifacts can arise due to the high reflectance due to the acoustic property difference of 10 (v_sapphire=10× speed of sound in water and >10× for speed of sound in tissue), as a high reflectance to low reflectance boundary leads to constructive interference that can impair visualization of the precise location of the tip. By superimposing ultrasound imaging with photoacoustic imaging, the active region at the distal end region of the fiber can be imaged to high accuracy. If ultrasound imaging is employed with a sufficiently high frequency, it can be possible in some cases to also image cancerous tissue due to increased vascularization within a tumor region. For example, the projection the acoustic signals generated by PIRL tissue disruption onto acoustically imaged tumor tissue, as highlighted by phase contrast and ultrasound image contrast agents (bubbles or nanoparticles), can provide intraoperative guidance for facilitating accurate positioning of the fiber and the appropriate delivery of therapy to the tissue of interest.

The use of ultrasound imaging is limited in depth resolution by acoustic attenuation that varies quadratically with frequency. To have sufficient spatial resolution to image the fiber position, ultrasound wavelengths of approximately 30 MHz (or approximately 30 micron acoustic wavelengths) may be beneficial in enabling near diffraction limited resolution with current transducer technology. This frequency limits the imaging depth to approximately 1-2 cm. This depth can be extended to approximately 5 cm by employing lower frequencies, such as frequencies between 10-20 MHz.

In order to provide accurate position tracking and guidance deeper within the body cavity, other imaging modalities that have less contrast may be employed to identify the position of the fiber in the 3D space of the body cavity. For example, fluoroscope x-ray imaging or use of electromagnetic field gradients or MRI may be employed to image the fiber and guide its position. Such imaging modalities may not have sufficient contrast to identify the fiber position relative to critical components within the body, to guide the fiber along the optimal, surgically relevant, pathway.

In such cases, as well as a general feature of imaging the fiber within the body, the position of the distal tip can be uniquely determined by exploiting the large strain field generated by the PIRL process at the fiber tip. The action of PIRL in tissue disruption via the ultrafast thermal energy deposition in the 10 micron scale leads to thermally driven volume expansion with strain fields (delta V/V) of 10-2 or larger. This strain field is orders of magnitude larger than that radiated by piezoelectric transducers used for the ultrasound imaging. This extremely large strain can be detected by conventional photoacoustic detection to give an extremely bright acoustic point source or beacon to uniquely position the fiber in the body. The extremely large magnitude of the strain field means that the signal can be detected up to the desired 5-10 cm depth, or deeper, that would then effectively give unique photoacoustic location of the fiber anywhere in the body. This acoustic beacon, which is inherently generated through the action of the PIRL pulses emitted by the fiber in the act of tissue disruption to make a pathway or for deliberate tissue disruption and thermally driven apoptosis, can be superimposed on other previously generated images such as CT scans, MRI, or use of position sensing devices (e.g. electrostatic location of the fiber tip). In the latter case, a pick-up coil can be used to map and display the fiber position relative to a 3D CT scan or other reference pre-operative volumetric image (provide that the preoperative image can be represented in the intraoperative reference frame, for example, by the use of stereotactic patient tracking devices, such as optical tracking systems, and/or via intraoperative image fusion, such as ultrasound to CT image fusion). The photoacoustic beacon thus provides a means of locating the fiber tip and its relation to the planned surgical route to the tissue of interest.

Once the distal end of the optical fiber is positioned within an intracorporeal tissue region of interest, such as a tumor, the optical fiber may be employed to transmit PIRL pulses with sufficient energy to disrupt a desired volume of tissue, for example, via one or more pulses, optionally with timed intervals suitable to achieve a desired volume of tissue disruption and/or killing of a specific volume of tissue. For example, after having reached the target site using a laser fluence appropriate for piercing the tissue, the energy and/or power of the infrared laser pulses may be increased to accelerate tissue disruption at the tip, and the tip can be scanned to increase the volume of disruption at the tumor site to eradicate the diseased tissue.

It will be understood that the skilled artisan may perform experiments with real or simulated tissue (e.g. a phantom) to determine a suitable pulse energy, number of pulses, and or pulse repetition rate to achieve a suitable level of PIRL-based disruption and/liquification, and/or thermal therapy induced apoptosis. For example, in the case of PIRL pulses tuned to the OH stretch water resonance at a wavelength of include 2.7-3.3 μm, each pulse interaction above the threshold for tissue disruption has been found to disrupt and liquify approximately 10-100 micron depth profile (depending on the chosen wavelength and the mechanical properties of the tissue) from the optical fiber exit face.

Once the optical fiber has penetrated a tissue region of interest via PIRL-induced tissue deformation and liquification, thermal diffusion from the fiber tip location can be exploited within a defined volume element to cause thermal-induced apoptosis. The optical fiber can be placed at a desired location in the target tissue and thermal diffusion can be exploited to deliver energy, e.g. either by continued irradiation with the PIRL laser or use of a WDM in fiber system to use any other wavelengths, such as 532 nm (green) light to deposit energy through other absorption bands (such as, for example, hemoglobin absorption bands in blood) to heat the tissue to apoptosis, providing a well-defined kill zone for cancer or eradication of other diseased tissue.

Tissue necrosis can be achieved with programmed temperature variation (delta T) within a controlled zone. Depositing known powers to heat the tissue and raise the temperature of the desired boundary, for example, up to 60° C., can be employed to kill the tissue. At this temperature, cells undergo “programmed cell death” or apoptosis. For most applications, simple diffusion models can be used to accurately determine the required power and time of exposure to lead to cell apoptosis out to a desired tissue diameter. An optical temperature sensor, of which many various designs exist, such as a fluorescence monitoring, phase interference monitoring or reflectometery including a Bragg grating written into the tip of the fiber can be incorporated in the distal end of the waveguide, which can monitor the localized temperature in-situ during the photothermal exposure to ensure proper irradiation protocols to induce apoptosis out to the desired tissue volume.

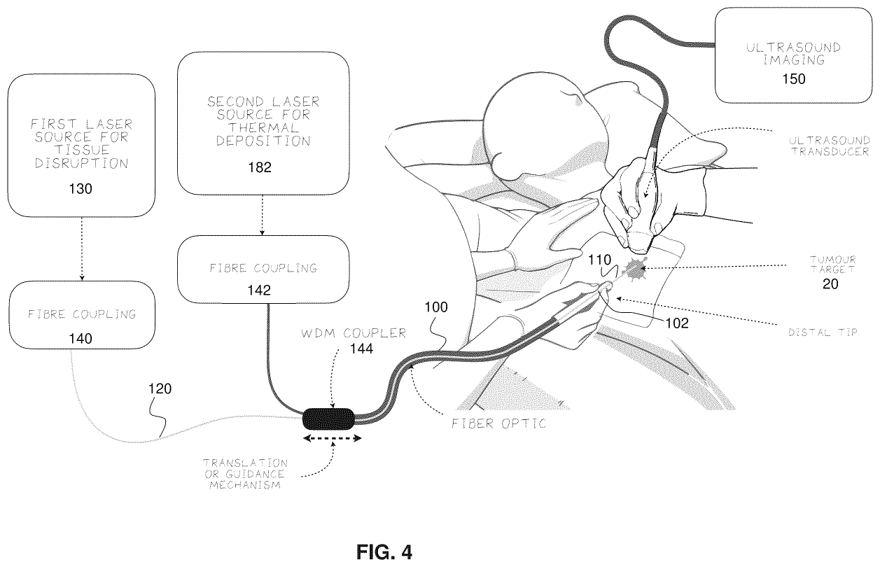

FIG. 4 illustrates an example system for PIRL-based local tissue disruption and thermal therapy. An optical fiber 120 delivers PIRL pulses from a PIRL laser system 130 and is received within and extends within an elongate body 100. PIRL pulses may be coupled from the laser source into the optical fiber 120 via coupling 140 (which may be a rotary optical joint to facilitate rotation of the optical fiber relative to the PIRL laser system 130; alternatively, an optical rotary joint may be housed within or on the elongate body 100). An additional light source 182, capable of delivering laser energy suitable for intratumoral thermal therapy (thermally-mediated apoptosis), is coupled to the optical fiber 120, for example, though a fiber coupling 142 and a wavelength division multiplexing coupler 144. In some example embodiments, the optical fiber will be provided within and extended from an assembly that includes multiple sections of supporting enclosures (support bodies and microcannulas). In some cases, the fiber itself may be encapsulated within a metal or plastic cannula that sits within a larger metallic cannula (a nested assembly). The fiber may protrude from the final section of this assembly bare for some distance depending on the fiber thickness and coatings.

As shown in the figure, the optical fiber 120 is received and supported by the elongate body 100. A distal microcannula 110 extends from the distal end 102 of the elongate body. In some example implementations, the distal portion of the optical fiber 120 resides at or near the distal end of the microcannula 110, or is extendable relative to the distal end of the cannula. In other example implementations, the distal microcannula 110 supports a distal optical waveguide that is in optical communication with the distal end of the optical fiber 120.

It will be understood that the optical fiber 120 may be formed from two or more segments. In some example implementations, at least a portion of the microcannula 110 (such as a distal potion) is flexible.

The distal microcannula 110 is aligned by the surgeon (or via robotic surgical subsystem) for angular placement and direct advancement to the target tissue 20, facilitated by PIRL-based tissue disruption and liquification that causes minimal collateral damage to neighbouring tissue along the path to the target tissue 20. Initial angular alignment may be provided by the distal microcannula portion 110 with an accurately determined initial position at the body's entry point and forward trajectory for the target tissue, involving geometric location and delivery, assisted with an imaging and/or positioning subsystem (i.e., a “navigation” or “guidance” system) 150. After having employed PIRL pulses to facilitate the penetration of the tumor by the distal tip of the optical fiber, and after having positioned the distal tip of the optical fiber at a desired location, the second laser source 182 is controlled to deliver thermal therapy, optionally repositioning the distal tip of the optical fiber at different intratumoral locations during the delivery of thermal therapy.

While the example implementation illustrated in the figure demonstrates the use of ultrasound-based image guidance, it will be understood that any suitable image guidance system, tracking system, or positioning system may be employed to facilitate guidance of the distal end of the microcannula to the target, optionally also providing surgical guidance based on intraoperative volumetric image data that is rendered in an intraoperative frame of reference and presented on a user interface.

As shown in the figure, the present system for intratumoral or intracavity direct drug injection includes the PIRL laser system 130 that is coupled to the optical fiber such that the output of the distal end of the optical fiber would have sufficient conditions (wavelength, pulse duration and intensity, as described above) to facilitate highly localized micro-disruption of tissue. In some example implementations, the pulse duration and fluence, and advancement speed may be selected based on the specific tissue type, or feedback signals such as force or temperature sensing.

As noted above, the position of the optical fiber 120, or distal optical waveguide, or distal region of the microcannula 110, may be detectable by and displayed, optionally relative to pre-operative image data, via a surgical guidance or navigation system, which can include a positioning mechanism, manual or motorized, which guides the insertion of the distal end of the apparatus into the body towards a targeted volume of tissue to be disrupted by the laser (disrupted tissue) within the boundaries of a solid tumor target. The position of the distal end of the optical fiber, distal waveguide, and/or cannula can be determined, for example, using a position sensor in conjunction with spatial imaging such as an ultrasound, x-ray or MRI.

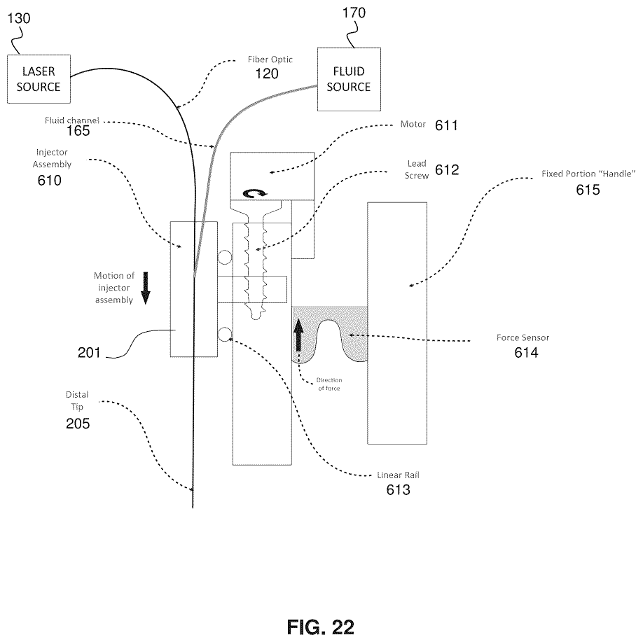

In some example embodiments, a force sensor may be incorporated between a static proximal portion of the apparatus (which is configured to reside outside of the body) and the distal portion that is configured for intracorporeal advancement into the tissue (e.g. using a motorized mechanism as described above). The signal from the force sensor may be used to adjust one or more of (i) the rate of movement of the distal end of the elongate conduit into the tissue, (ii) the power of the laser, (iii) the repetition rate of the laser, and (iv) the pulse energy of the laser, during the piercing phase of the process. An example of such an embodiment is shown in FIG. 22, which shows a schematic representation of an example embodiment which includes motorized motion for the piercing action and force sensor for monitoring of the frictional forces for optimal piercing speed and laser parameters.

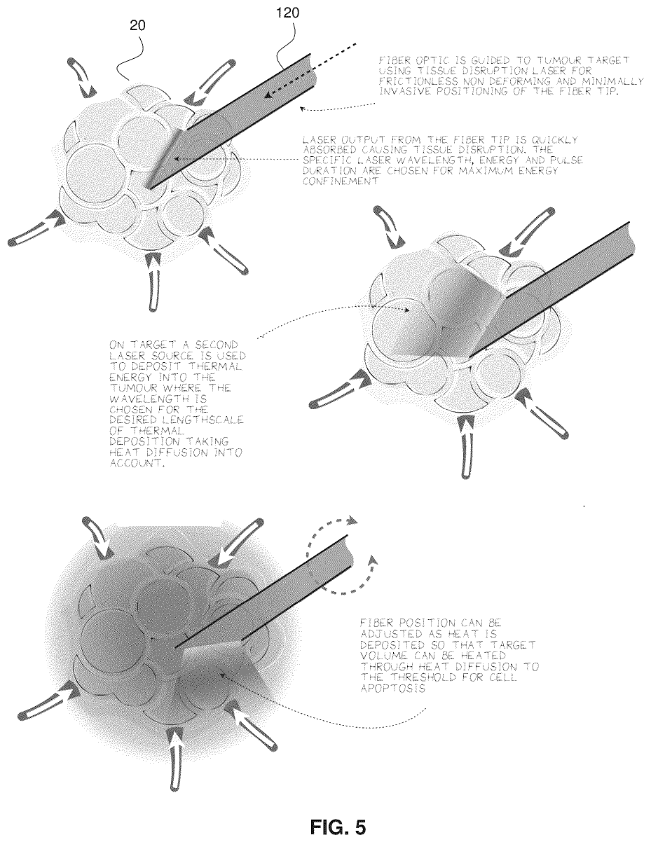

FIG. 5 illustrates the delivery of thermal therapy within a tumor 20 via the same optical fiber 120 that was employed to achieve intratumoral access via the delivery of PIRL laser pulses. After having positioned the distal tip of the optical fiber within the tumor via PIRL-mediated intratumoral access, a second laser (e.g. the laser system 182 in FIG. 4) is employed to deliver thermal therapy.

The wavelength of the second laser may be selected based on a desired length for thermal deposition, taking thermal diffusion into account. For example, near infrared 750 nm CW lasers may be employed, which absorb over approximately 5 mm deep in tissue, or green lasers such as 550 nm which absorb over approximately 0.5 mm in tissue. In some example implementations, lasers with outputs at 532 nm can be used to deposit energy into the target tissue by the absorption of light in oxygen transport pigments such as hemoglobin or myoglobin for heavily vascularized cancer tissues to have preferential absorption in the cancerous tissue and tunable wavelengths to adjust the absorption depth for energizing the target tissue to the desired temperature for apoptosis at the optimal heating rate, taking into account thermal diffusion, to more efficiently target cancerous tissue.

As shown in FIG. 5, the distal tip of the optical fiber may be repositioned and/or reoriented during the delivery of thermal therapy, for example, according to a surgical plan, to provide a desired spatial distribution and dose of thermal energy. For example, e.g. using pre-operative volumetric image data to estimate the tumor size, and known or estimated thermal conduction and diffusive transport properties of the tumor, and a known or estimated absorption depth of the second laser for a given absorption band, and a system that can use this information to determine a set of positions and orientations to ensure a requisite temperature rise, for a prescribed time interval and dose of laser exposure, to achieve apoptosis.

In some example implementations, an optical fiber delivery system delivers pulsed infrared pulses to provide a pathway for injection of liquid therapeutic agents (e.g. drugs) with minimal collateral damage to the adjacent tissue while simultaneously enable tissue disruption at the fiber exit, thereby creating pathways for drug delivery and elimination of pressure gradients common to highly vascularized cancer tissue that otherwise leads to outflow of the drug and loss of efficacy.

Some example embodiments of the present disclosure facilitate local injection of a pharmaceutical fluid (e.g. containing a therapeutic agent; a drug) to improve effectiveness of the injected drugs, for example, for tumor destruction or other actions for medical treatments, beyond what is possible for needle stock drug delivery. For example, embodiments of the present disclosure can address the problem of chemo-immunity due to the presence of high interstitial fluid pressure, reduce the pressure by tissue disruption, and facilitate the uniform dispersal of the drug at the specific targeted site for optimal drug delivery. Some example embodiments of the present disclosure can therefore be employed to reduce the amount of drug needed to selectively attack cancer tissue, for example, by orders of magnitude, and thereby facilitate a reduction in the side effects of chemotherapy. The present methods may therefore lead to an improvement in the quality of life, potentially to near pre-cancer status, by reducing the associated side effects of chemotherapy. In some example embodiments, after drug delivery, the application of radiation or energy delivery may be employed to create barriers to physical diffusion of the drug away from the cancer site, thereby potentially further increasing efficacy. The pharmaceutical fluid may be a liquid, vapour, gas or combination thereof.

Accordingly, in some aspects, the present disclosure facilitates the selective delivery of liquid therapeutic agents to cancerous tissue, even for high osmotic pressure tissues, and provides a means to block diffusion of the liquid therapeutic agent away from the cancer site, thereby solving the problem of uptake vs. diffusion away from the cancer, even for normal osmotic pressure conditions. Furthermore, in some aspects, the present disclosure additionally or alternatively facilitates the site-selective delivery of a liquid therapeutic agent or other material to a given location in the body without damage to the surrounding tissue in the act of the drug delivery. As noted above, some aspects of the present disclosure also enable the subsequent blocking of diffusion from the injection site to solve the problem of both high osmotic pressure that blocks entry of drugs to cancer sites and diffusion away from the site once injected. The methods of the present disclosure may avoid or reduce the dependency on systemic drug regimens by enabling access to cancer sites for site selective drug delivery. This capability can reduce the required dose, for example, by orders of magnitude, thereby avoiding side effects associated with chemotherapy.

Such embodiments of the present disclosure may be employed to avoid or reduce the need for high selectivity drugs, as is currently needed according the present standard of care treatment methods that rely on systemic treatment. Instead, for example, a drug can be delivered that is suitable for rapid uptake locally, optionally combined with means to block subsequent diffusion with the creation of barriers to diffusive transport away from the drug delivery point. The ability to directly and locally target tumor cells while they reside within the tumor may avoid the need to also target migratory cancer cells that lead to metastasis of the cancer.

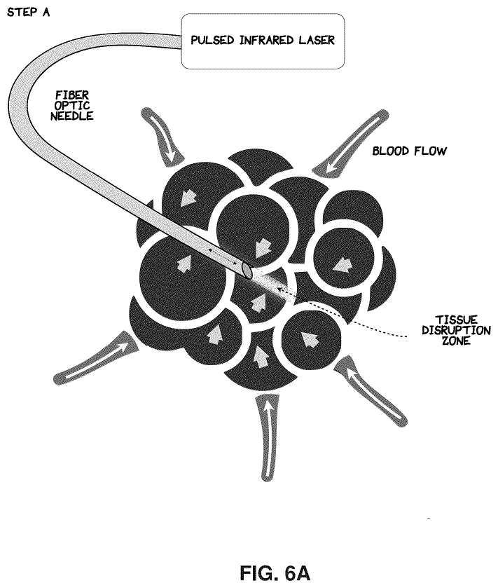

FIGS. 6A and 6B schematically illustrate the direct injection of a drug into a tumor via initial pulsed infrared laser pulse mediated tissue disruption. In step A, an optical fiber needle disrupts a zone of solid tumor tissue, thereby reducing interstitial fluid pressure by piercing/deflating. As shown in FIG. 6B (step B), a drug can be subsequently injected under lower pressure, such that the drug remains localized within the tumor with less diffusion. The process illustrated in FIGS. 6A and 6B is performed in two steps involving the initial delivery of the pulsed infrared laser pulses via first optical delivery device and the subsequent injection of drug into the optically disrupted tumor via a second drug injection device. In other example implementations, the optical disruption of the tumor and the subsequent delivery of the drug is performed using a single integrated optical and fluidic delivery device, as described in further detail below.

The third challenge noted above, namely the need to avoid diffusion of an intratumorally injected therapeutic agent, can be addressed by the selection of specialized compounds or by the additional combination of activation of light activated compounds, for example, using the same laser delivery fiber to transmit into the tumor additional light sources specifically designed for favourable drug interaction or photo-adhesion. Additional radiation can be applied at suitable wavelengths, pulse durations, and interaction times to cause localized photo-activation of a drug. The drug can be modified specifically for the purpose of light-induced fixation (photocleaving a blocking group to create reactive states that physically fix the drug to the cancer site). For example, in some example implementations, chemotherapy drugs can be employed with a means to physically fix the drug or to enhance uptake faster than diffusion away from the cancer site.

The PIRL tissue disruption and liquification mechanism avoids excessive shear force during entry of the device into tissue, thereby enabling rapid healing with greatly reduced wound entry trauma. In the case of fiber delivery, the cross-sectional wound is the size of the fiber itself, which can be on the order of 200 microns or smaller down to several tens of microns in diameter by tapering the end of the fiber, similar to the dimensions of a single cell and smaller than any acupuncture needle and where the pulsed infrared laser output from the distal tip of the instrument obviates the need for applied mechanical force. Accordingly, a fiber configured to emit infrared pulses can enter anywhere in the body with minimal trauma. As noted above, the wound size can be reduced to the diameter of approximately 10-20 cells without shear damage to the tissue, such that the tissue elastically rebounds upon exit of the fiber to leave minimal damage.

This elimination or significant reduction of trauma also means this approach can be considered for use even for metastasized cancer, as there is no or minimal additional penalty to the patient with respect to recovery. In normal cancer surgeries, large incisions are needed with massive disruption and mechanical displacement of healthy tissue to access a cancer site. According to the example methods of the present disclosure, there could be effectively no limit to the number of procedures that could be done as long as the cancer can be imaged and its spatial location determined. Such an approach renders it practical to deliberately create tissue damage by local photoactivated drug binding of a blocking agent, specific to the cancer drug, to keep the drug physically located within the cancer site by increasing the barrier to drug diffusion away from the target site, and optionally, for example, to deliver photothermal energy to achieve conditions well above thermal denaturation to the point of inducing coagulation or cauterization (with aeration).

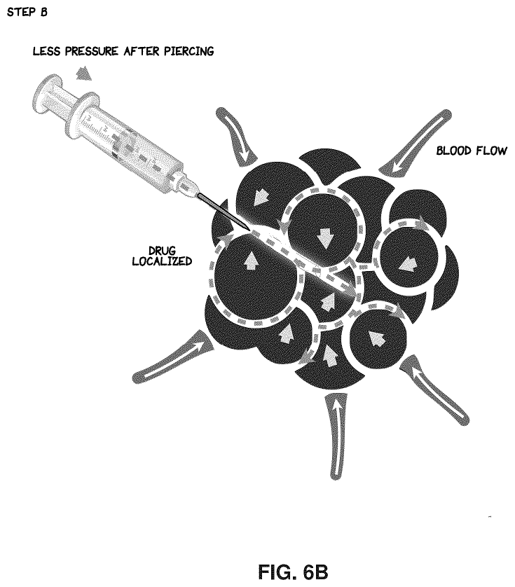

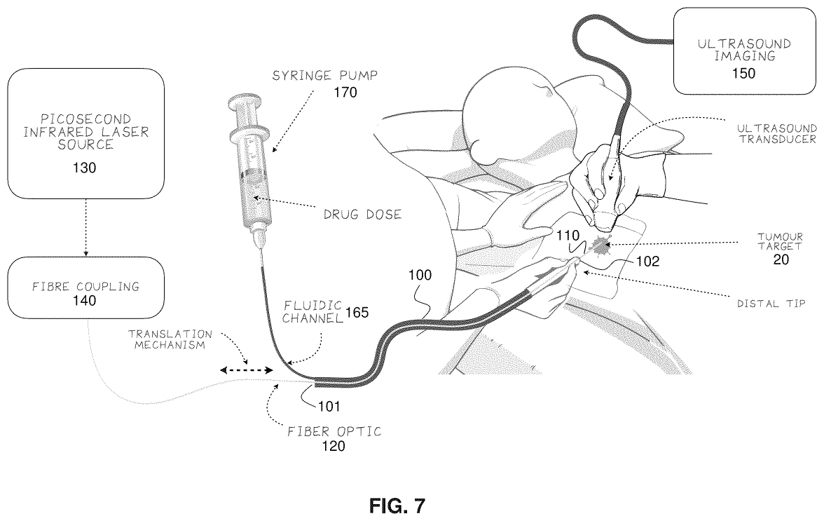

Accordingly, in some example embodiments, an integrated optofluidic device is provided that includes an optical fiber for the delivery of PIRL laser pulses to achieve tissue disruption and a fluid delivery conduit for the delivery of a pharmaceutical fluid and/or to perform aspiration. Referring now to FIG. 7, an example system is illustrated for performing PIRL-based tissue disruption of a tumor and the subsequent delivery of drug to the disrupted tumor. Similar to the example system shown in FIG. 4, the figure shows a handheld device having an elongate body 100 with a proximal end 101 and a distal end 102. An optical fiber 120 delivers PIRL pulses from a PIRL laser system 130 and is supported by and extends through the elongate body 100. PIRL pulses may be coupled from the laser source into the optical fiber 120 via coupling 140 (which may be a rotary optical joint to facilitate rotation of the optical fiber relative to the PIRL laser system 130; alternatively, an optical rotary joint may be housed within or on the probe body 100). Example embodiments of other suitable system configurations are disclosed in International Patent Application No. PCT/CA2023/050929, titled “SYSTEMS, DEVICES AND METHODS FOR TARGETED TISSUE THERAPY” and filed on Jul. 11, 2023.

As shown in the figure, the optical fiber 120 is received and supported by the elongate body 100. A microcannula 110 extends from the distal end 102 of the elongate body. In some example implementations, the distal portion of the optical fiber 120 resides at or near the distal end of the microcannula 110, or is extendable relative to the distal end of the microcannula. In other example implementations, the microcannula 110 supports a distal optical waveguide that is in optical communication with the distal end of the optical fiber 120. It will be understood that the optical fiber 120 may be formed from two or more segments, and that at least the distal portion of the microcannula 110 may be flexible.

The present example system for intratumoral or intracavity direct drug injection includes the PIRL laser system 130 that is coupled to the optical fiber such that the output of the distal end of the optical fiber would have sufficient conditions (wavelength, pulse duration and intensity, as described above) to facilitate highly localized micro-disruption of tissue, substantially reducing the interstitial fluidic pressure enabling the direct and local injection of a liquid therapeutic agent. As noted above, the position of the optical fiber 120, or distal optical waveguide, or distal region of the microcannula 110, may be detectable by and displayed, optionally relative to pre-operative image data, via a surgical guidance or navigation system.

FIG. 7 also illustrates an example implementation in which a fluid delivery conduit 165 is provided, for example, to facilitate delivery of a drug or other fluid to the to the distal end of the microcannula 110. As shown in the example embodiment illustrated in the figure, fluid is supplied to the fluid delivery conduit 165 by a source of fluid (e.g., a fluid reservoir) and a pump. In the example implementation shown in FIG. 7, fluid is delivered to the fluid delivery conduit 165 by a syringe pump 170. The fluid delivery conduit 165 is interfaced with, or extends within, an internal lumen within the probe body 100, such that fluid within the fluid delivery conduit 165 is dispensable from the distal end of the microcannula 110, or from a fluidic channel formed in an optical waveguide that extends from, or is extendable from, the distal end of the microcannula 110. The syringe pump 170 controls the injection of a controlled dose of a pharmaceutical compound (drug) chosen for treatment of the tissue target.

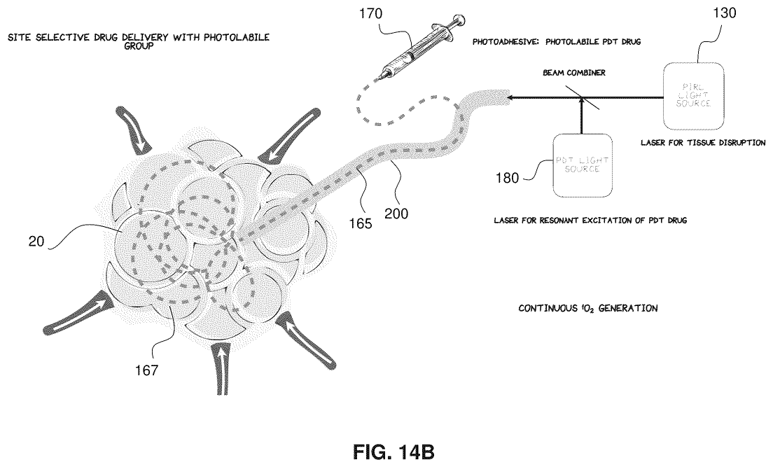

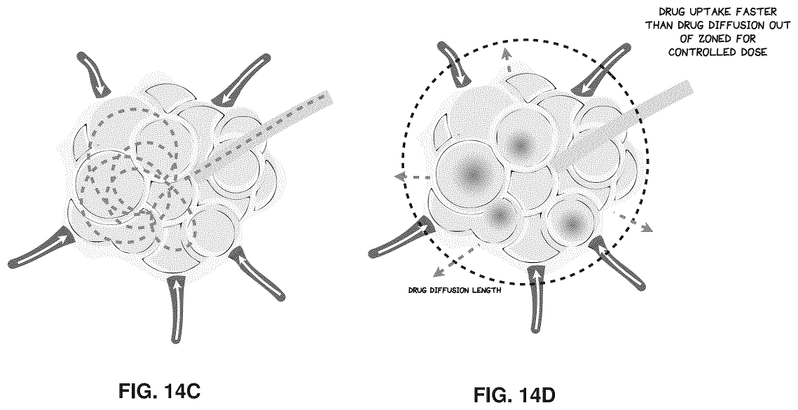

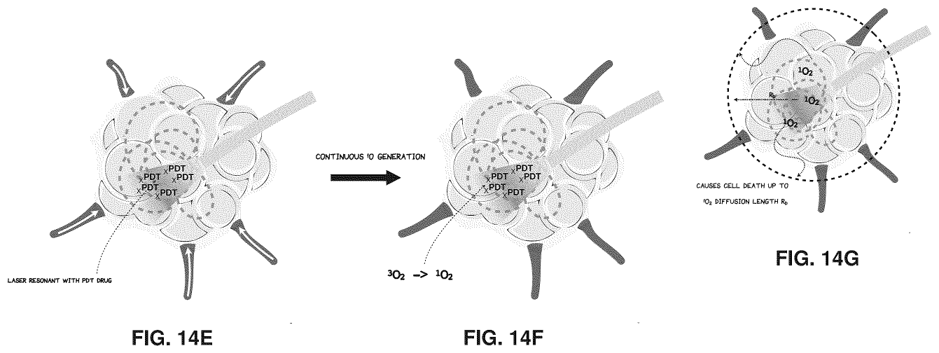

In some example embodiments, the system shown in FIG. 7 may include an additional laser source that is coupled to the optical fiber 120 and employed to deliver optical energy for additional therapy, such as, for example, to deliver local thermal therapy prior to or after having injected the therapeutic agent within the intracorporeal region of interest. An example of such an augmented system is shown in FIG. 14A and described in further detail below.

For example, a dual treatment modality involving initial local thermal therapy and subsequent local delivery a therapeutic agent may be beneficial in that the local delivery of the therapeutic agent augments the deposition of laser energy to thermally kill cancer cells, further increasing the volume sampled and subject to cancer drug regimes with a high degree of selectivity to rapidly growing cancer cells and are known to conserve cell constituents to enable reabsorption of the cancer cells as typically observed as shrinkage of the tumor. The latter effect provides a dual hammer to kill cancer with higher confidence levels, and allow the cancer tissue to be reabsorbed, to lead to maximal recovery of tissue function without dead tissue blocking functions.

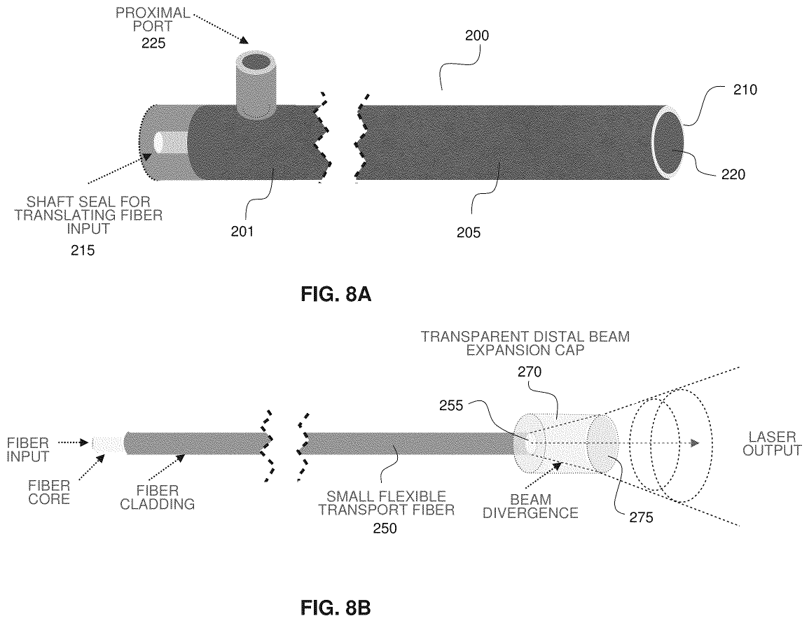

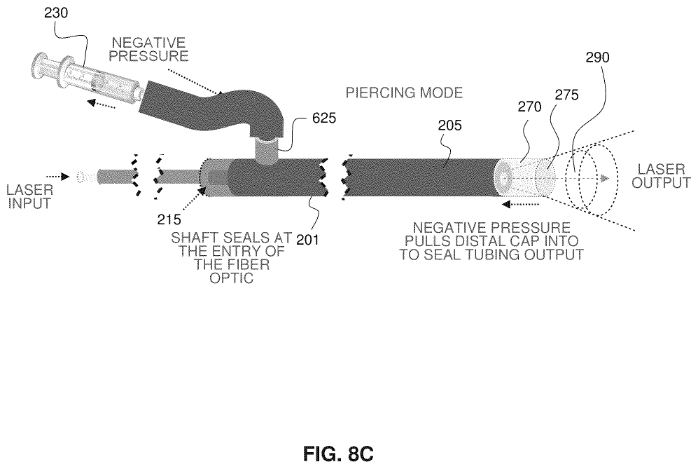

The following sections of the present disclosure, and the corresponding drawings in FIGS. 8A-8C, 9A, 9B, 10A, 10B, 11A-11G and 12A-B describe and illustrate several non-limiting example embodiments of an intracorporeal laser pulse delivery assembly that includes a microcannula housing an optical fiber for PIRL-based tissue disruption and a fluidic channel for dispensing a pharmaceutical fluid and/or performing aspiration, where at least the microcannula portion of the device is readily suitable for intracorporeal insertion. Such assemblies, which can be employed and adapted according to the various example system configurations and applications described elsewhere in the present disclosure, such as, but not limited to, guidance and positioning (e.g. ultrasound, photoacoustic, electromagnetic detection), treatment modality (e.g. laser-based disruption and liquification of tissue, thermal therapy, and photodynamic therapy), are referred to as “intracorporeal laser pulse delivery assemblies”, “fine needle assemblies” or “laser-assisted fine needle assemblies”, and provide example intracorporeal laser pulse delivery systems that employ laser-assisted (laser-mediated) tissue disruption during needle insertion to facilitate a minimally invasive approach to performing a wide range of applications, including local delivery of a pharmaceutical fluid, fine needle biopsy, and other intracorporeal interventions described elsewhere in the present disclosure, and adaptations and variations thereof. For example, many of the present example embodiments that relate to the internal delivery of a pharmaceutical fluid enable the use of small volumes of pharmaceutical fluids and the reduction of dead volume relative to conventional devices.

FIG. 13 shows an example method for performing drug delivery through a multiple step procedure according to an example drug delivery apparatus. In step 1, substep A, an optical fiber 120, coupled to the laser, is positioned within a rigid or flexible cannula, 800, that includes a mounting fixture or handle, 805 and also a fluidic seal, 215, that allows insertion and rotation and or translation of cylindrical components such as the fiber assembly while preventing escape of fluid from the cannula distal end. As shown in substep B, with the laser activated, the cannula 800 and optical fiber 120 are advanced together towards the target in a controlled way. In step 2, substep C, with the cannula 800 fixed in place, the optical fiber 120 can be advanced (and/or optionally rotated if a curved distal section, 810 is included) and laser pulses are delivered into a volume of the tissue, where the size and shape of this volume is controlled by motion of the optical fiber 120 relative to the cannula 800. As shown in substep D, once sufficient laser pulses have been delivered to create a zone of liquified tissue, 815, the optical fiber 120 is retracted from the cannula 800, the latter of which remains in place once the optical fiber 120 is completely retracted, as shown in substep E. Next, as shown in step 3, substep F, an empty syringe 820 is inserted into the cannula 800 through the fluidic seal 215, and the liquified tissue is aspirated through the syringe. As shown in substep G, the elasticity of the tissue closes or reduces the volume of the liquified zone (e.g. the remaining void) after the aspiration step. Once a desired or sufficient volume of liquified tissue has been aspirated, the aspiration syringe 820 is removed, leaving the cannula 800 in position and the seal 215 maintains the pressure inside the distal portion of the cannula 800. In step 4, substep J, a drug loaded syringe 830 is inserted into the cannula 800 and a volume of drug fluid (e.g. liquid pharmaceutical) 835 is injected at a controlled rate, to refill the void from where the laser liquified tissue 815 had been aspirated. Once the void is filled (e.g. with a desired volume/quantity of the drug), additional liquid volume can be injected, for example, at a rate that matches transport/diffusion into the tissue, as shown at substep L. After injection is complete, as shown in substep M, the cannula 800 and the injection syringe 830 can be removed.

A pharmaceutical fluid, as used herein, refers to a liquid that can have diagnostic and/or therapeutic uses. For example, in some example implementations, a pharmaceutical fluid may include a therapeutic agent, such as an active pharmaceutical ingredient. A pharmaceutical fluid may additionally or alternatively include one or more components including, but not limited to, an excipient, a carrier, an adjuvant, a stabilizer, a solubilizer, and diagnostic reagent. A pharmaceutical fluid may include a component selected from various classes of drugs utilized for therapeutic purposes, encompassing traditional pharmaceuticals, also nanoencapsulated drugs, liposomes, micelles, dendrimers, polymeric nanoparticles, nanocrystals, and solid lipid nanoparticles. Additionally, a pharmaceutical fluid may include a component selected from photoactivated drugs, ultrasonically activated drugs, and drugs for gene therapy. Gene therapy drugs encompassing gene editing tools, viral vectors, non-viral vectors, antisense oligonucleotides, small interfering RNA, and messenger RNA therapeutics, and also from transfection agents like lipofectamine, calcium phosphate, polyethylenimine, and vaccines of various types such as live attenuated vaccines, inactivated vaccines, subunit vaccines, recombinant vector vaccines, DNA vaccines, and mRNA vaccines. A pharmaceutical fluid may include a component selected from biological drugs, or biologics, consist of monoclonal antibodies, recombinant proteins, fusion proteins, therapeutic enzymes, and blood factors. Also, pharmaceutical fluid may include a component selected from radioactive pharmaceuticals including diagnostic contrast agents, prodrugs designed to metabolize into active forms in the body. A pharmaceutical fluid may a component selected from targeted therapies, immunomodulatory drugs, antimicrobial agents, cardiovascular drugs, psychotropic drugs, respiratory drugs, gastrointestinal drugs, hormonal drugs, nutraceuticals, and diagnostic agents further diversify the pharmacological landscape.

Many of the example embodiments disclosed herein employ a laser-assisted fine needle device having a distal microcannula portion having a diameter, configured for intracorporeal insertion (penetration and/or delivery to an intracorporeal tissue target) that is less than 1 mm, less than 0.8 mm, less than 500 μm, or less than 300 μm, with a minimum distal device diameter of, for example, 50 μm, 60 μm, 80 μm, 100 μm, 150 μm, 200 μm, or 250 μm, optionally employing a tapered optical fibers mid-assembly or near the tip to further reduce diameter and friction. Smaller sizes are also possible, for example, using optical fibers which can be fabricated even on sub-micron scales (reference for a 800 nm diameter sapphire fiber https://www.sciencedirect.com/science/article/pii/S0167577X14017522) For example, the intracorporeal laser pulse delivery device may have a distal device diameter with a gauge (Birmingham) greater than 20, 22, 24, 26, 28 or 30 or higher. This small device diameter can play an important role in achieving intracorporeal insertion with reduced friction, and can also be beneficial in reducing tissue damage during insertion, providing a significant improvement over previous technologies.

FIGS. 8A, 8B and 8C illustrate an example intracorporeal laser pulse delivery assembly configured for laser-assisted tissue disruption. The intracorporeal laser pulse delivery assembly includes an elongate conduit (e.g. sheath, cannula) 200, shown in FIG. 8A and an optical fiber assembly shown in FIG. 8B. The elongate sheath 200 has an inner lumen through which a solid-core optical fiber 250 extends to form the integrated assembly shown in FIG. 8C. The elongate conduit 200 has a distal opening 220 at a distal end 210 thereof, with a distal microcannula portion 205, the distal opening 220 being in fluid communication with the inner lumen.

As illustrated in FIG. 8C, the optical fiber 250 passes through a proximal valve 215 of the elongate conduit 200, and travels through the elongate conduit. The proximal valve 215 is configurable to form a fluidic seal the optical fiber, while permitting longitudinal movement of the optical fiber. An example of such a movable valve/seal is a Tuohy-Borst seal/valve, which provides a secure, adjustable connection that prevents leakage and can allow control over the movement (extension/retraction) of the optical fiber 250 while preventing fluid leakage. Non-limiting examples of other suitable valve/seal mechanisms include o-ring seals, piston rings, gland seals, lip seals, cup seals, rod seals, chevron seals, U-cup seals, buffer seals. In some example embodiments, the proximal valve mechanism may be actuated to removably lock the position of the optical fiber in place, thereby providing a fiber “braking mechanism” such as a locking piston seal, brake piston seals, which can immobilize the piston in a specific position either mechanically or using hydraulic or pneumatic pressure, or a locking/braking Tuohy-Borst valve.

The elongate conduit 200 may be formed from a wide variety of materials and may be flexible or rigid. For example, thin walled metallic tubing made from materials such as stainless steel, nitinol, titanium. Various types of thin-walled plastic tubing typically utilized in medical devices, including polyethylene (PE) tubing, polyvinyl chloride (PVC) tubing, polyurethane (PU) tubing, silicone tubing, polytetrafluoroethylene (PTFE) tubing, nylon tubing, polyethylene terephthalate (PET) tubing, fluorinated ethylene propylene (FEP) tubing, and thermoplastic elastomer (TPE) tubing. May also include capillary tubing, made from glass or ceramic materials.

As shown in FIG. 8C, a distal beam expansion optical element 270 is coupled to a distal end 255 of the optical fiber 250 that facilitates beam expansion of the infrared pulses emerging from the distal end of the optical fiber. For example, the distal beam expansion optical element 270 may be made from various optically transmitting materials such as various glasses, including silica, low-OH silica, fluoride glass, sapphire, quartz, and also ceramic materials that transmit the specific wavelengths of light. This may also include optical elements formed from materials such as GRIN lenses, ball lenses, volume Bragg gratings, and also waveguides including fiber optics with and without cladding. The distal beam expansion optical element 270 is secured to the distal end of the optical fiber 250 such that the distal beam expansion optical element 270 is mechanically supported by the optical fiber and resides beyond or in contact with a distal opening 220 of the elongate conduit 200, and such that the infrared laser pulses propagate from the optical fiber 250 into the distal beam expansion optical element 270, and expand within the distal beam expansion optical element 270 to substantially fill a distal forward-facing surface 275 of the distal beam expansion optical element 270. The divergence angle of the light emerging from the fiber is approximately equal to the acceptance angle of the beam expansion optic, thereby ensuring that the emitted light fills the distal forward-facing surface without significant overfill or underfill. As used herein, the phrase “substantially fills the distal forward-facing surface” means that the optical intensity at the radially outermost portion of the distal forward-facing surface is at least 10% of an optical intensity within the center of the distal forward-facing surface, and that at least 80% of the pulse intensity is directed onto the distal forward-facing surface. The distal beam expansion optical element 270 may be secured to the distal end of the optical fiber, for example, by fusion splicing, diffusion bonding, optical grade UV-cured adhesive bonding, epoxy bonding, silicone bonding, acrylic bonding, hot melt adhesive bonding, glass fusing, soldering, and hybrid bonding techniques.

The optical fiber 250 has a proximal end coupled to a pulsed infrared laser source, such that the infrared laser pulses are delivered through the optical fiber, expand through the distal beam expansion optical element 270, and pass through the distal forward-facing surface 275 of the distal beam expansion optical element 270. The infrared laser pulses are delivered with pulse conditions such that a pulse energy, duration, wavelength and fluence delivered to tissue residing adjacent to the distal forward-facing surface 275 of the distal beam expansion optical element 270 facilitates the direct local liquification and homogenization of the tissue, as described above.

The distal forward-facing surface 275 of the distal beam expansion optical element 270 is sufficiently large (e.g. has a sufficient diameter) such that a lateral extent of the laser-irradiated tissue volume 290 residing beyond the distal beam expansion optical element 270 exceeds a diameter of a distal end of the elongate conduit 200, thereby facilitating further insertion of the distal portion of the elongate conduit 200 into the laser-irradiated and liquified tissue volume 290. In other words, the distal beam expansion optical element 270 is sized to generate a disruption effect that is larger in diameter than the distal portion of the elongate conduit, such that reduced or minimal resistance is encountered during piercing and intracorporeal insertion through tissue while irradiating the distal tissue region with the laser pulses.

As shown in FIG. 8C, a proximal portion of the distal beam expansion optical element 270 has a shape configured to close and seal the distal opening 220 of the elongate conduit 200 when the optical fiber 250 is retracted through the proximal valve 215. Likewise, extension of the optical fiber 250 such that the distal beam expansion optical element 270 moves distalward relative to the distal end 210 of the elongate conduit 200 facilitates fluidic communication between the inner lumen and an external region beyond the distal opening 220. Accordingly, the movable distal beam expansion optical element 270 and the distal opening 220 of the elongate conduit 200 form a distal valve.

As shown in FIGS. 8A and 8C, the elongate conduit 200 includes a proximal port 225 that is located remote from a distal end of the elongate conduit 200 (i.e. the proximal port is accessible outside the body during intracorporeal insertion of the elongate distal portion 205 of the needle assembly) and is shown within a proximal portion 201 of the elongate conduit 200. As the outer diameter of the optical fiber 250 is less than a diameter of the inner lumen, a fluid delivery channel is formed between the outer surface of the optical fiber and the inner surface of the elongate conduit 200, with the consequence that the proximal port 225 is in fluid communication with the distal opening 220 of the elongate conduit 200. FIG. 8C illustrates how the application of a negative pressure to the inner lumen of the elongate conduit 200, through the proximal port 225, provides a suction force that can assist in maintaining the distal beam expansion optical element 270 to remain in contact with the distal end 210 of the elongate conduit 200, thereby closing the distal opening 220 of the elongate conduit and closing the distal valve. While FIG. 8C illustrates the use of a manual syringe as an example of a pump mechanism, it will be understood that any suitable pump mechanism can be employed to regulate pressure within the inner lumen. Non-limiting examples of suitable pump mechanisms include peristaltic pumps, piston pumps, diaphragm pumps, gear pumps, rotary vane pumps, plunger pumps, micropumps, gear-diaphragm pumps, peristaltic syringe pumps, and magnetic drive pumps, microfluidic pumps. Akin to a syringe pump, these offer precise control and delivery of small liquid volumes.

As described above, the example intracorporeal laser pulse delivery assembly illustrated in FIGS. 8A-8C enables the laser-assisted disruption of tissue encountered during insertion of the distal portion 205 of the through tissue to a target site within the body, thereby facilitating an atraumatic insertion path through the body to the target site with a reduction in friction.

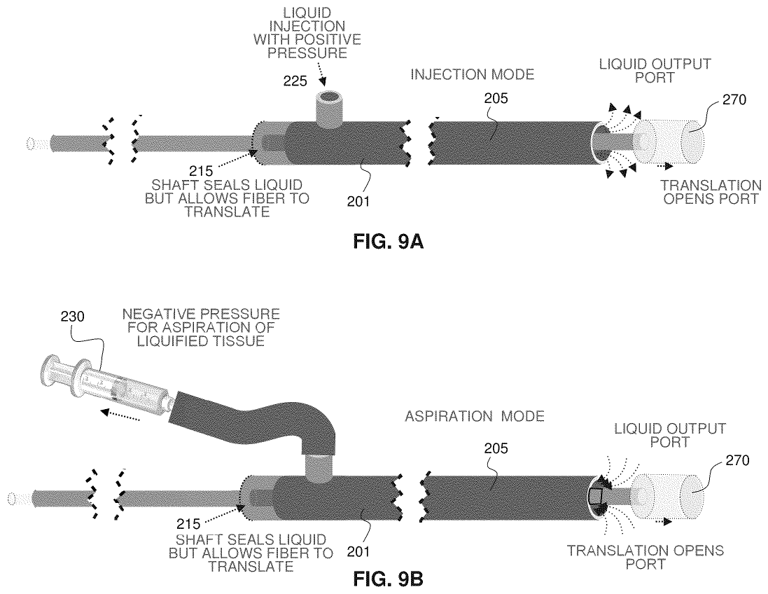

As shown in FIG. 9A, the opening of the distal valve via the extension of the optical fiber 250 such that the distal beam expansion optical element 270 is moved beyond the distal end 220 of the distal portion of the elongate conduit 205 brings the inner lumen and the proximal port 225 into fluid communication with an external region beyond the distal end 210 of the elongate conduit, thereby enabling the dispensing of a liquid, such as a pharmaceutical fluid, to an intracorporeal target site when the distal end of the intracorporeal laser pulse delivery assembly is inserted into or positioned adjacent to the target site. As explained elsewhere in the present disclosure, local tissue at the target site may be disrupted (liquified) by delivery of the infrared laser pulses prior to dispensing of the pharmaceutical fluid. This disruption of the local tissue region residing beyond the distal end of the device may reduce an interstitial fluid pressure and facilitate injection of the pharmaceutical fluid with less pressure and resistance.

In some example embodiments, the intracorporeal laser pulse delivery assembly may be configured to perform an intracorporeal dispensing procedure such that the pharmaceutical fluid emerges close to the distal tip of the optical fiber, while ensuring an overall diameter of the needle assembly, within the distal region of the needle assembly, that is not substantially larger than the diameter of the optical fiber. Accordingly, such an intracorporeal laser pulse delivery design can reduce the invasiveness of tissue penetration, minimizing damage and improving the procedure's overall efficacy.

For example, the example intracorporeal laser pulse delivery assembly shown in FIG. 8C may be employed to dispense a pharmaceutical fluid to an intracorporeal target site as follows. The intracorporeal laser pulse delivery assembly may be prepared, prior to intracorporeal insertion, such that a pharmaceutical fluid resides within at least a distal portion of the inner lumen of the elongate conduit 200, thereby avoiding the injection of air during subsequent intracorporeal dispensing (i.e. priming the assembly with either the pharmaceutical fluid or an additional ‘buffer’ liquid). With the distal valve residing in a closed state (i.e. the distal beam expansion optical element 270 closing the distal opening 210 of the elongate conduit 200, optionally in combination with the application of a negative pressure via the pump mechanism, ensuring that the assembly stays intact and without premature leakage of the fluid containing the therapeutic agent), the pulsed infrared laser source is controlled to perform local tissue disruption and liquification while at least an elongate distal portion of the elongate conduit is inserted into the body, such that the distal end of the elongate distal portion 205 is positioned proximal to or within an intracorporeal target site, with the local tissue disruption and liquification facilitating the insertion process.

As explained above, closure of the distal valve may be facilitated, at least in part, by the application of a negative pressure by the pump mechanism, resulting in a suction force that draws the distal beam expansion optical element 270 into contact with the distal end 210 of the elongate conduit 200. Closure of the distal value may alternatively be facilitated by a force caused by withdrawal of the optical fiber 250 through the proximal valve 215. The optical fiber 250 is then extended to move the distal beam expansion optical element 270 away from contact with the distal opening 220, thereby opening the distal valve (optionally in combination with the application of a positive pressure via the pump mechanism). The pump mechanism is then controlled to dispense the pharmaceutical fluid beyond the distal opening 220 into the external region that is adjacent to or within the intracorporeal target site, with the dispensing of the pharmaceutical fluid being illustrated in FIG. 9A.

In another example implementation, the example intracorporeal laser pulse delivery assembly shown in FIG. 8C may be employed to perform needle biopsy at an intracorporeal target site as follows. The optical fiber 250 is retracted to contact the distal beam expansion optical element 270 with the distal opening and close the distal valve, (optionally in combination with the application of negative pressure via the pump mechanism). The pulsed infrared laser source is then controlled to perform local tissue disruption and liquification while inserting the elongate distal portion 205 of the elongate conduit 200 into the body and positioning the distal end of the elongate distal portion of the elongate conduit proximal to or within the intracorporeal target site. The optical fiber is then moved in a distalward direction, through the proximal valve 215, to extend the distal beam expansion optical element 270 away from contact with the distal opening 220 and open the distal valve. The pump mechanism is then controlled to aspirate tissue residing proximal to the distal opening 220. As explained elsewhere in the present disclosure, local tissue at the target site may be disrupted (liquified) by delivery of the infrared laser pulses prior to aspiration of the tissue.

The proximal valve 215 may, in some example implementations, be actuated to lock the position of the optical fiber 250 relative to the elongate conduit 200, to maintain the distal valve in an open configuration during aspiration of the tissue, thereby preventing the distal beam expansion optical element from being drawn back into contact with the distal opening 220. In other words, the proximal valve 215 may incorporate a mechanical brake permits the entire assembly to advance for tissue penetration, but also enables movement of the fiber assembly relative to the fluidic assembly for opening the distal valve and maintaining the distal valve in an open state, using the brake, during the application of negative pressure through the inner lumen, so that tissue can be aspirated without closing of the distal valve.

In some example implementations, the mechanical aspiration mechanism illustrated in FIG. 9B may enable the control of both the delivery of pharmaceutical fluids and the aspiration or extraction of tissue, in a coordinate manner, enabling these processes to be finely tuned according to the needs of the procedure.

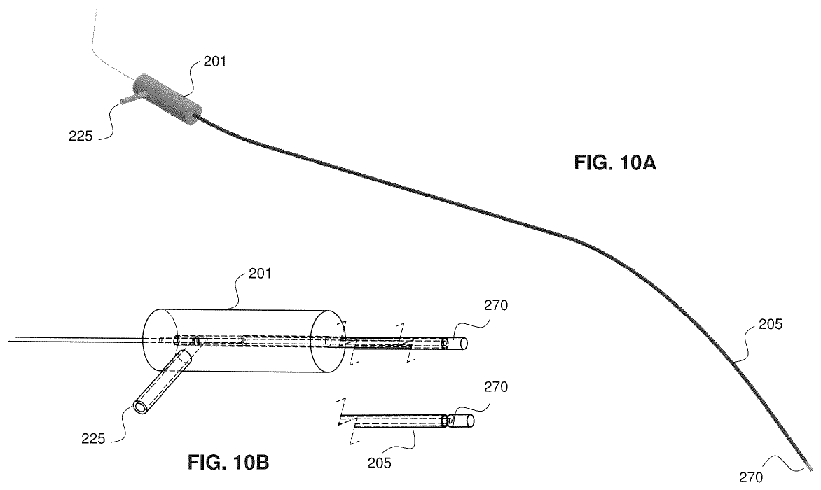

As shown in FIG. 10A, the intracorporeal laser pulse delivery assembly may include a proximal housing 201 that includes the proximal port 225 and the proximal valve (not shown), and the elongate conduit 200 (a needle portion) that has a small diameter (as described above) configured for intracorporal insertion. FIG. 10B shows the internal structure of the example proximal housing 201, showing the distal valve in closed and open states via extension of the distal beam expansion optical element 270 relative to a distal end of the distal portion elongate conduit 205.

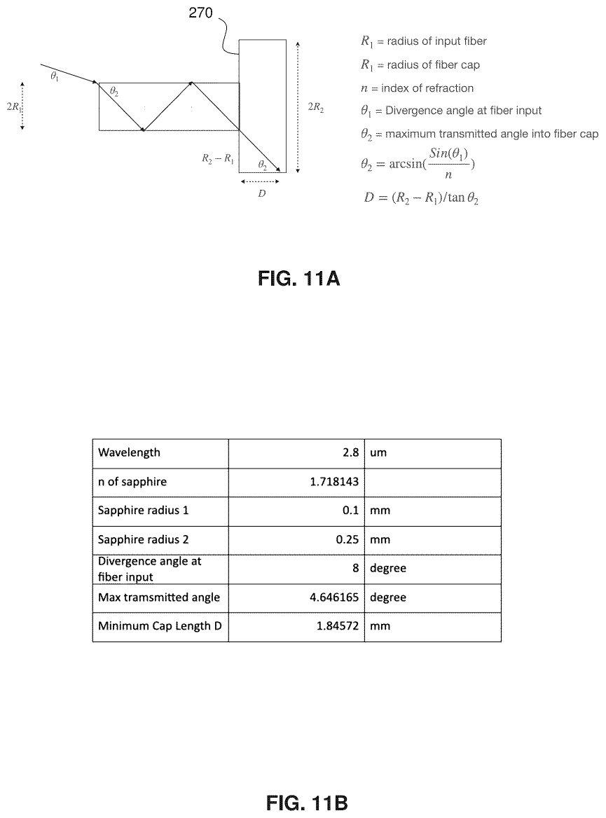

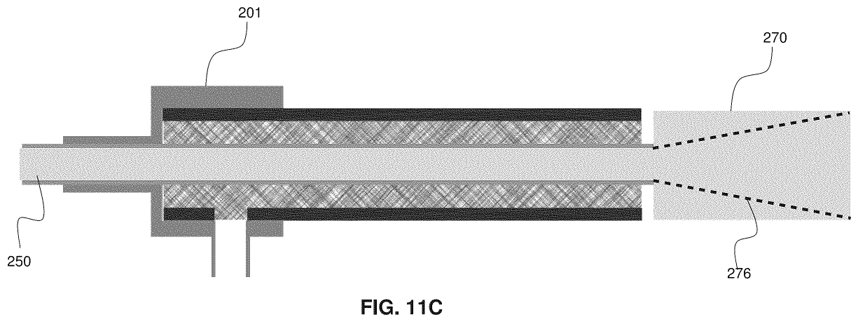

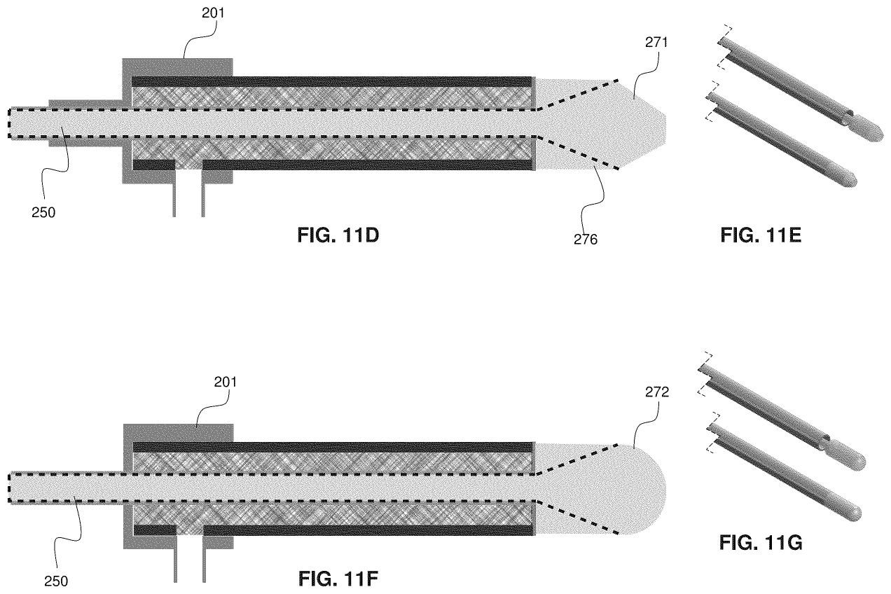

FIGS. 11A and 11B show example design schematics for an example embodiment of the distal beam expansion optical element 270 configured to expand the emerging optical beam from the optical fiber to fill a 500 micron diameter distal output aperture that approximately matches the diameter of the outer sheath (not shown) housing the optical fiber.

While FIGS. 8B and 8C show an example embodiment in which the distal beam expansion optical element 270 has a cylindrical shape, it will be understood that such a shape is not intended to be limiting and that other shapes for the distal beam expansion optical element 270 can be employed in the alternative, provided that the proximal portion of the distal beam expansion optical element is shaped to facilitate closure of the distal opening of the elongate conduit when the optical fiber 250 is retracted.

For example, non-limiting alternative shapes for forward-looking distal surface of the distal beam expansion optical element 270 may include conical, spherical or aspheric shapes, which can increase the laser-assisted insertion (penetration) of the intracorporeal laser pulse delivery assembly by further reducing the force required for insertion. FIGS. 11C-11G provide cross-sectional and isometric views of example intracorporeal laser pulse delivery assemblies having different shapes of the distal beam expansion optic 270, showing tapered 271 and rounded 272 configurations which can be optimized for frictionless laser-assisted tissue piercing and clearance (the longitudinal dimension is not to scale). The figures also show the proximal port 225, the optical fiber 250, and the expansion of the laser beam 276 within the distal beam expansion optic 270. The reduction of the friction force, facilitates an even more seamless and less invasive entry into the tissue, provided that the distal beam expansion optical element 270 allows the laser pulses to diverge and substantially fill the forward-facing distal surface. The laser pulses parameters upon penetration of the tissue should be sufficient to disrupt the tissue, preferably from a single pulse or a short train of pulses, to facilitate disruption and liquification, for example as disclosed above, and described in U.S. Pat. No. 11,648,150 with regard to variations in distal tip geometry.

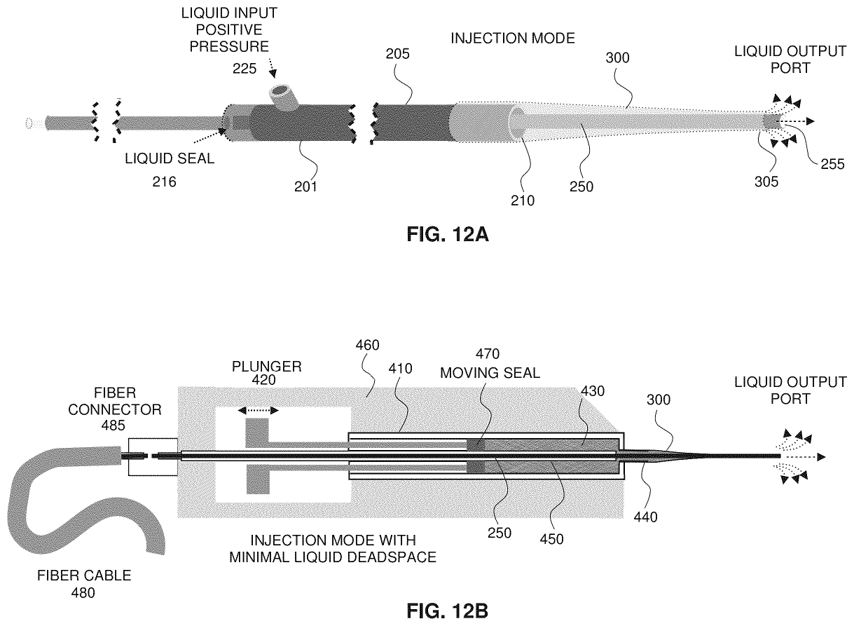

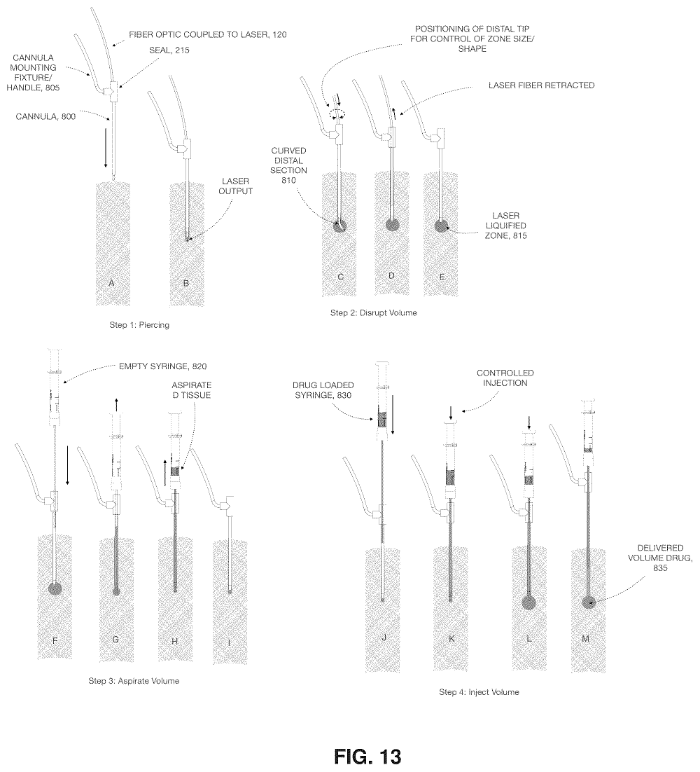

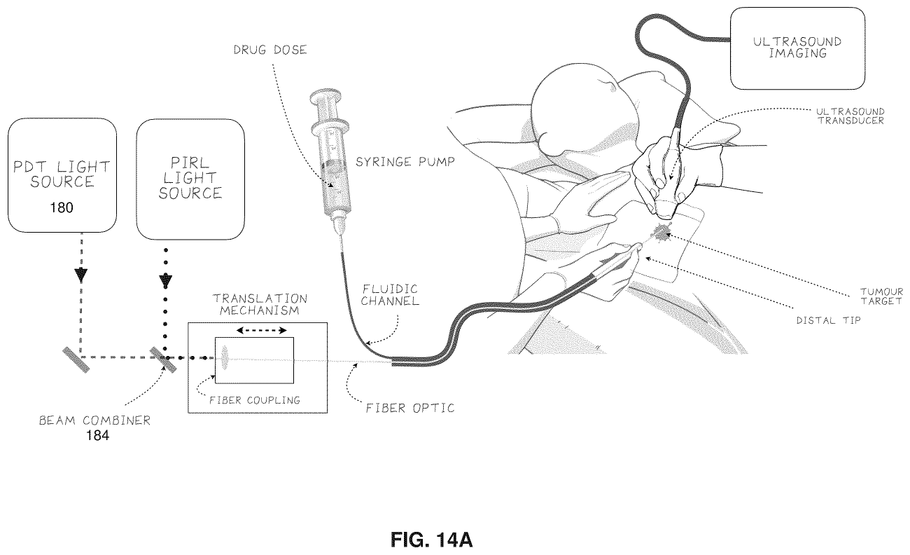

Referring now to FIG. 12A, an alternative example laser-assisted intracorporeal laser pulse delivery assembly is disclosed for dispensing a pharmaceutical fluid to an intracorporeal target site. In a manner similar to the embodiment shown in FIGS. 8A to 8C, the intracorporeal laser pulse delivery assembly of FIG. 12A includes an elongate conduit 200 having a proximal end, an inner lumen, and distal opening in fluid communication with the inner lumen. The elongate conduit 200 also includes a proximal valve 215 and a proximal port 225, where the proximal port is in fluidic communication with the inner lumen. As in the previous example embodiments, an optical fiber 250, having a proximal end coupled to a pulsed infrared laser source, passes through the proximal seal 216 into the elongate conduit and extends through the elongate conduit. In the present example embodiment, the proximal seal 216 need not permit movement (extension or retraction) of the optical fiber 250.

A distal portion of the optical fiber 250 extends beyond the distal end 210 of the elongate conduit 200 and passes through the inner lumen of a sheath 300, such that a distal end 255 of the optical fiber resides at or distally adjacent to a distal end 305 of the sheath. The sheath 300 extends distalward from the distal end 210 of the elongate conduit and has a proximal portion overlapping with and secured to an outer surface of the elongate conduit 200 near the distal end 210 of the elongate conduit. An inner lumen of the sheath 300 is in fluid communication with the inner lumen of the elongate conduit 200.

As shown in FIG. 12A, in some example implementations, the sheath 300 may have a tapered profile, such that a distal portion of the sheath contacts and applies a passive compressive force to an outer surface of the optical fiber 250. The passive compressive force establishes a distal valve that is passively closed in the absence of pressure applied to the proximal port 225. However, it will be understood that in general, the distal portion of the sheath need not apply a compressive force to the optical fiber, and in some cases may reside adjacent to the optical fiber with a gap therebetween, such that the device is absent of a distal valve per se.

A pump mechanism (not shown in FIG. 12A) is provided in fluid communication with the proximal port 225. The pump mechanism is capable of dispensing a fluid (e.g. a liquid) through the distal end of the sheath 300. In cases in which the distal portion of the sheath is conformal to the fiber and forms a passive distal valve, the pump mechanism is capable of dispensing a fluid via application of sufficient pressure to overcome the passive compressive force. FIG. 12A illustrates the dispensing of a pharmaceutical fluid from the distal end 305 of the sheath 300, proximal to the distal end 255 of the optical fiber 250.

The intracorporeal laser pulse delivery assembly of FIG. 12A may be assembled, for example, by first securing the sheath 300 to the distal end portion of the elongate conduit 200, with the sheath initially having a cylindrical profile with a diameter that exceeds that of the optical fiber 250. The optical fiber is then fed through the proximal seal 216 and extended through the elongate conduit and the sheath. Heat may then be applied to the sheath, such that sheath shrinks to take on a tapered profile and contact at least the distal portion of the optical fiber to form the passive distal valve.

The sheath may be formed from a wide variety of materials, including, but not limited to, heatshrinking material such as thin walled PTFE, PVC, PET or FEP heatshrink tubing or other elastomeric or rigid tubing materials made of various biocompatible plastics, thin walled metal tubing and or thin walled glass or ceramic capillary tubing.

The sheath has a wall thickness that is sufficiently thin such that the distal elongate region of the intracorporeal laser pulse delivery assembly has an outer diameter that is less than 1 mm, less than 0.8 mm, less than 500 μm, less than 300 μm, less than 200 μm, or less than 100 μm, with a minimum distal device diameter of, for example, 50 μm, 60 μm, 80 μm, 100 μm, 150 μm, 200 μm, or 250 μm. In other example implementations, the sheath may have a wall thickness that is sufficiently thin such that the distal elongate region of the intracorporeal laser pulse delivery assembly has an outer diameter that for example, less than 200 μm, or less than 100 μm, with a minimum distal device diameter of, for example, 50 μm, 60 μm, or 80 μm. For example, the intracorporeal laser pulse delivery device according to the embodiment illustrated in FIG. 12A may have a distal device diameter with a gauge (Birmingham) greater than 20, 22, 24, 26, 28 or 30. In some example implementations, the wall thickness of the sheath may be less than 50 μm and even 25 μm in the case of some heat shrink materials where the wall thickness is a function of the applied heat.

The example intracorporeal laser pulse delivery device shown in FIG. 12A, and variations thereof contemplated by the present disclosure, introduce a significant variation relative to the embodiment shown in FIGS. 8A-8C by incorporating a thin-walled structure that surrounds the optical fiber, facilitating fluid travel around the perimeter of the optical fiber and allowing for the dispensing of the liquid pharmaceutic close to the distal end of the optical fiber. This design aims to streamline the drug delivery process by incorporating a thin-walled tapered structure, designed to be minimal in size but effective in guiding the liquid pharmaceutic around the optical fiber and acts to bridge the diameter difference between the fluidic assembly and the optical fiber. The present example embodiment also enables perimeter liquid pharmaceutic travel, i.e. by allowing the fluid to travel around the perimeter of the optical fiber, this present example embodiment ensures that the fluid can be delivered precisely and efficiently to the target area. Finally, the present example embodiment facilities the exit (dispensing) of the liquid pharmaceutic close to the distal end (tip) of the optical fiber, where the strategic placement of the liquid exit near the distal end of the optical fiber ensures that the pharmaceutical fluid is delivered directly to the intended site, reducing or minimizing dispersion and potentially increasing treatment efficacy.

Moreover, unlike the example embodiment illustrated in FIGS. 8A-8C, the optical fiber of the embodiment illustrated in FIG. 12A does not require a distal beam expansion optical element, because the additional diameter associated with the thickness of the sheath can be very small (e.g. heat shrink plastic tubing with a wall thickness of less than 25 μm). It is noted that in some example variations of the embodiment illustrated in FIG. 12, the optical fiber can have a tapered diameter, or a shape of tip such as angled or conical that further reduces piercing forces while the laser pulses are delivered and the intracorporeal laser pulse delivery assembly is inserted. This present example embodiment also simplifies the assembly by allowing the pharmaceutical fluid to flow directly to the tip of the optical fiber without any moving parts. The present example embodiment is therefore streamlined for drug delivery, and helps to ensure that therapeutic agents are delivered precisely and efficiently to the target site.

FIG. 12B illustrates yet another alternative example intracorporeal laser pulse delivery assembly for dispensing a pharmaceutical fluid to an intracorporeal target site. Unlike the embodiment illustrated in FIG. 12A, the example embodiment illustrated in FIG. 12B includes an integrated liquid dispensing mechanism, shown in the figure in the form of an integrated syringe that includes a syringe barrel 410 and a plunger (piston) 420, forming an internal chamber 430 that houses a pharmaceutical fluid.

In the example embodiment illustrated in FIG. 12B, an optical fiber 250 extends beyond the distal end 440 of the syringe barrel 410 and passes through the inner lumen of a sheath 300, such that a distal end of the optical fiber resides at or distally adjacent to a distal end of the sheath, in a manner similar to the embodiment shown in FIG. 12A. The sheath 300 extends distalward from the distal end 410 of the syringe barrel, the sheath having a proximal portion overlapping with and secured to an outer surface of the syringe barrel 410 near the distal end 440 of the syringe barrel. An inner lumen of the sheath 300 is in fluid communication with the inner chamber 430 of the syringe.

Furthermore, as in the example embodiment illustrated in FIG. 12A, the sheath 300 may have a tapered profile, such that a distal portion of the sheath contacts and applies a passive compressive force to an outer surface of the optical fiber 250. The passive compressive force establishes a distal valve that is passively closed in the absence of pressure applied to the proximal port 225.

A proximal end of the optical fiber 250 is coupled to the pulsed infrared laser source (not shown) such that the infrared laser pulses are delivered through the optical fiber. A proximal portion of the optical fiber is supported by a rigid elongate support 450, and is optionally connected to the pulsed infrared laser source via an external optical fiber cable 480 and an optical fiber connector 485.

The rigid elongate support 450 is rigidly supported relative to the syringe barrel 410 by a rigid body 460 (e.g. a support frame, housing, handle). The rigid elongate support 450 passes through a moving seal 470 defined within a central region of the movable plunger 420, into the inner chamber 430. The difference between the inner diameter of the syringe barrel 410 and the outer diameter of the rigid elongate support 450 determines, at least in part, the volume capacity of the chamber 430 when the plunger is retracted. Accordingly, the volume of the chamber 430 can be selected by a suitable choice of these diameters. In some cases the total volume should be as small as 0.001 ml-0.01 mL (1-10 μL) or larger depending on the amount of the pharmaceutical agent to be delivered. The diameter and length chosen to match the required amount of drug to be delivered.

The moving seal 470 (e.g. a gasket, or types of moving piston seals listed earlier) is configured to form a fluidic seal with the rigid elongate support 450 while the movable plunger 420 is moved relative to the rigid elongate support 450. This configuration ensures that the optical fiber remains rigidly supported during actuation of the plunger 420 to dispense the pharmaceutical fluid out of the distal end of the sheath, adjacent to the distal end of the optical fiber, as the distal passive valve formed by the compression of the sheath against the optical fiber is opened under applied fluidic pressure from the syringe.

A benefit of the present example embodiment, having an integrated syringe, is the ability to control and reduce the dead space associated with pharmaceutical fluid that remains undelivered or is required to prime the assembly, thereby enabling improved drug delivery efficiency and reduced waste. This reduction of dead volume, is accomplished, for example, by integration of the syringe into the assembly, as the fluidic channel starts inside the syringe chamber where the pharmaceutical fluid is stored. This approach avoids the need for additional dead volume caused by connection channels to an external chamber which holds the liquid. The integration of the stationary optical fiber with a movable plunger is also beneficial in that the optical fiber remains fixed in place while the plunger moves to create positive pressure, reducing the fluid chamber size and forcing the fluid through the narrow-walled tubing around the perimeter of the optical fiber. This design ensures precise control over the delivery of the pharmaceutical fluid without the need to move the optical fiber.

An additional benefit is provided by the dual moving seal of the plunger, involving both a first seal inherent to the plunger, and the inner movable seal the moves relative to the fixed rigid elongate support that secures the optical fiber. The first seal allows the plunger to move and push the pharmaceutical fluid while sealing the inner chamber containing the pharmaceutical fluid, and the inner movable seal provides a seal against the optical fiber support. This ensures that the pharmaceutical fluid is directed exclusively towards the distal end of the optical fiber for efficient and targeted delivery to the tissue volume residing adjacent to the distal end of the needle assembly, and which has optionally been disrupted and liquified by the action of the infrared laser pulses. Moreover, when the plunger is fully depressed, the inner chamber is substantially emptied and only small fraction of residual pharmaceutical fluid remains in the small space around the perimeter of the optical fiber in the elongate distal region of the needle assembly.

Although the example embodiment shown in FIG. 12B was illustrated based on an implementation in which the elongate conduit included the distal sheath of FIG. 12A, it will be understood that in other example implementations, the distal portion of the assembly, residing distalward of the syringe barrel, may be provided according a wide range of elongate conduit configurations, such as any of those described herein, or alternatives or variations thereof.

The example intracorporeal laser pulse delivery devices, assemblies and systems of the present disclosure represent a potential paradigm shift in the local delivery of therapeutic agents within the body, providing an alternative method of therapeutic agent delivery that circumvents the aforementioned limitations of previous approaches. Firstly, the intracorporeal laser pulse delivery devices disclosed herein involve a substantial reduction in dead volume relative to conventional approaches, enabling the use of smaller volumes of pharmaceutical fluid with less wastage when locally delivering the pharmaceutical fluid to a target site within the body. Secondly, the forward-looking tissue disruption mechanism of the present intracorporeal laser pulse delivery devices clear a path for forward insertion of the device, significantly reducing the risk of tissue damage and easing the ability to advance the device to a desired target site within the body. Thirdly, the ability of the intracorporeal laser pulse delivery devices to locally disrupt pathological cells residing distalward from the distal end of the intracorporeal laser pulse delivery device enables the insertion of the intracorporeal laser pulse delivery device into a tumor without significantly reduced resistance due to the reduction or absence of intratumoral osmotic pressure, thus enabling the local delivery of a therapeutic agent and/or the aspiration of liquified tissue directly within a tumor.

In some example embodiments, as illustrated in FIG. 14A, and as compared for FIG. 7, an additional light source, shown as photodynamic therapy (PDT) light source 180 may be coupled into the optical waveguide (for example, using a beam combining element such as a free-space dichroic mirror 184 as shown, or for example, via a fiber based wavelength-selective coupler, not shown) for the purpose of photo-fixing the delivered drug compound to increase drug uptake and/or prevent rapid diffusion. In one example implementation of such an embodiment, the same distal tip shown would allow advancement of the optical fiber back to the target where the volume of the injected drug can be exposed to sufficient optical radiation for the desired photochemical outcome.

By positioning PDT drugs near the fiber optic tip, the disclosure enables localized drug delivery and subsequent activation by specific wavelengths of light emitted through the fiber. Once activated, the PDT drugs generate cytotoxic singlet oxygen in the surrounding tissue, which effectively targets and destroys diseased cells. The continuous creation of singlet oxygen is achieved by maintaining a controlled and sustained illumination from the fiber optic, allowing for ongoing drug activation and enhanced therapeutic efficacy in diseased tissue while minimizing damage to healthy surrounding areas. This dual-function system provides precise and localized control over both drug delivery and light exposure, significantly improving treatment outcomes.

Photodynamic therapy (PDT) drugs, also known as photosensitizers, come in various classes, each with unique properties tailored to specific clinical needs. First-generation PDT drugs, such as Photofrin (porfimer sodium), are porphyrin-based compounds that have been widely used in treating cancers and non-malignant conditions. These drugs absorb light at longer wavelengths, typically in the red region, and have a relatively long half-life in tissues, which can lead to prolonged photosensitivity in patients. Second-generation photosensitizers, like mTHPC (Temoporfin) and Verteporfin, are developed to have more precise targeting, shorter tissue retention times, and stronger absorption at wavelengths that penetrate deeper into tissues, thus enhancing their effectiveness for internal tumors. Additionally, there are chlorin-based photosensitizers, such as Talaporfin, which have higher singlet oxygen yields and faster clearance from the body. Third-generation PDT drugs focus on conjugating photosensitizers with targeting moieties such as antibodies or peptides, which allow for highly selective accumulation in cancer cells, minimizing damage to healthy tissues.

FIGS. 14B-14G illustrate the mechanism of laser assisted injection of photodynamic drugs, where the drug is designed for faster uptake than diffusion out of the tumor boundaries and the drug is activated by an additional light source, resonant with photolabile groups attached to the drug molecules to create a photoadhesive which will be bound in position while creating singlet oxygen for destruction of cancer cells. This latter step allows continuous monitoring of cancer shrinkage and adaptation of light levels for controlled dosing of singlet oxygen to adjust the cancer treatment as needed. FIG. 14B shows an elongate conduit 200 extending into a tumor 20, with entry facilitated by the delivery of PIRL pulses from PIRL light source 130. The figure also shows the use of a syringe 170 coupled to a fluid delivery channel 165 extending through the elongate conduit 200, and the dispensing of a drug 167 into the tumor 20, with the injected drug 167 shown in FIG. 14C. FIG. 14D shows how the uptake of the drug occurs on a timescale that is faster than diffusion of the drug out of the tumor for delivery of a controlled dose.

FIGS. 14E-14G show the fiber optic inside a solid tumour after injection of a photosensitizing drug, which remains inactive until exposed to a specific wavelength of light. Once the fiber optic is positioned inside the tumor, light is emitted from its tip at the specific wavelength required to activate the photosensitizer. Upon activation, the photosensitizer reacts with oxygen present in the tumor environment to produce highly reactive singlet oxygen, a cytotoxic species that induces cellular damage. This localized production of singlet oxygen leads to the selective destruction of tumor cells without significant harm to surrounding healthy tissue.

By positioning the fiber inside the tumor, the PDT treatment can create a highly focused area of singlet oxygen generation, allowing for the continuous and sustained treatment of the tumor from the inside out. As seen in FIG. 14F, this approach can help to facilitate exposure of the tumor cells in close proximity to the fiber optic to the maximum therapeutic effect, leading to efficient tumor cell death, inhibition of tumor growth, and potential tumor regression. FIG. 14G shows the ability of the fiber to deliver both light and liquid therapeutics further enhances treatment precision, ensuring that the drug is activated exactly where it is needed, minimizing side effects and damage to adjacent healthy tissue.

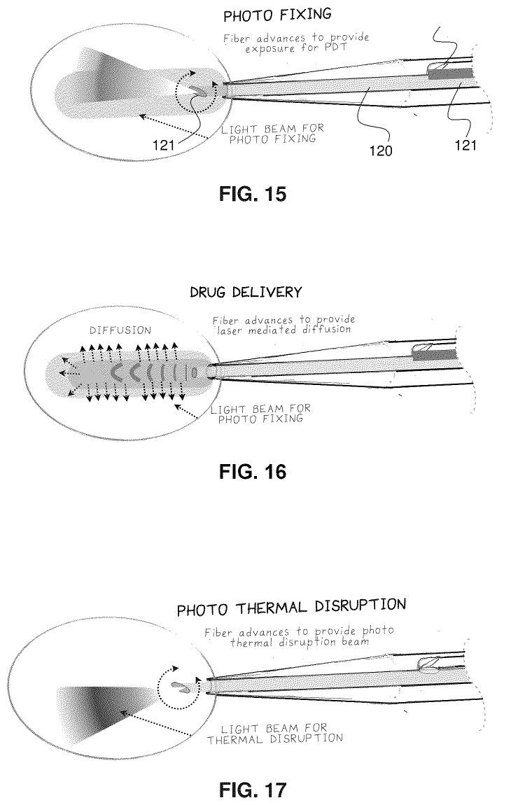

FIG. 15 illustrates an example embodiment in which the distal end 121 of the optical fiber 120 is angled to allow exposure to a wider volume of tissue through translation and rotation of the fiber end either independently or simultaneously with additional PIRL irradiation. Such an implementation may facilitate the delivery of laser light of a suitable wavelengths, pulse durations, and time durations to modify the surroundings of the targeted site to prevent diffusion of the drug away from the target. This step may involve, for example, photorelease of removable group on a particle drug, such as photodynamic therapy (PDT) drugs but not limited to PDT drugs, to lead to chemical bonding in place at the target site, i.e., block and fix the drug to the target site. The action of the drug in the case of photodynamic therapy can be controlled by light exposure using the optical fiber that was employed for initial disruption of the target tissue, or, for example, a replacement optical fiber using the same path. Another means to exploit light for blocking drug diffusion involves coagulating the surrounding area or cauterizing by higher power irradiation to physically block diffusion to enable real time monitoring and changes in treatment to reduce cancer volumes or ensure completely eradication of the cancerous tissue. In other example implementations, the distal end of the fiber may have other shapes that facilitate exposure to a wider volume of tissue through translation and rotation, such as a cone shape.

In some example implementations, as illustrated in FIG. 16, after drug delivery, the PIRL radiation may be provided to contact the dispensed drug fluid and generate a photomechanical force that improves diffusion of the pharmaceutical agent by creating large pressure gradients within the inside of the tumor through photomechanical cavitation effects.

In another example embodiment, as illustrated in FIG. 17, an additional laser source is provided for photothermal treatment of the target tumor, as previously described.

In another example embodiment, the example embodiments described above may be configured to obtain a liquified tissue microbiopsy sample.

In other example embodiments, the optical fiber employed for the delivery of PIRL pulses may be coupled to an external optical detection system that is configured to deliver, through the optical fiber, interrogating optical energy to the disrupted and liquified tissue, and to collect optical energy that is responsively emitted by the disrupted and liquified tissue. The disrupted and liquified tissue may reside beyond the distal end of the cannula. Alternatively, the disrupted and liquified tissue may reside within a distal portion of the cannula after retraction of the optical fiber to create a partial vacuum. Non-limiting example modalities for performing in-situ microbiopsy include spectroscopic methods such as Raman spectroscopy, fluorescence spectroscopy and frequency comb and laser induced breakdown spectroscopy. For example, Raman spectroscopy can be performed by employing the optical fiber used for PIRL disruption to also deliver excitation energy and collect backscattered Raman signals, which can be analyzed for biomarkers of disease or normal tissue. The very high excitation and thermal heating of tissue may also lead to light emission that provides a spectral signature or fingerprint of particular constituents of the tissue. Such an approach may be employed, for example, to determine whether or not the distal tip of the optical fiber resides within the tumor target (or another identifiable intracorporeal tissue region), and such analysis may be performed, for example, before, during or after the delivery of a given form of local therapy.

In other example embodiments, a fluidic channel employed for the delivery of a therapeutic agent, and/or an additional fluidic channel, can be interfaced with a pump (e.g. a mechanical pump or a syringe), and the pump can be controlled to aspirate one or more volumes of the disrupted and liquified tissue for biopsy analysis. This biopsy aspiration step may be performed before, during, or after the delivery of local therapy to the selected intracorporeal tissue region.

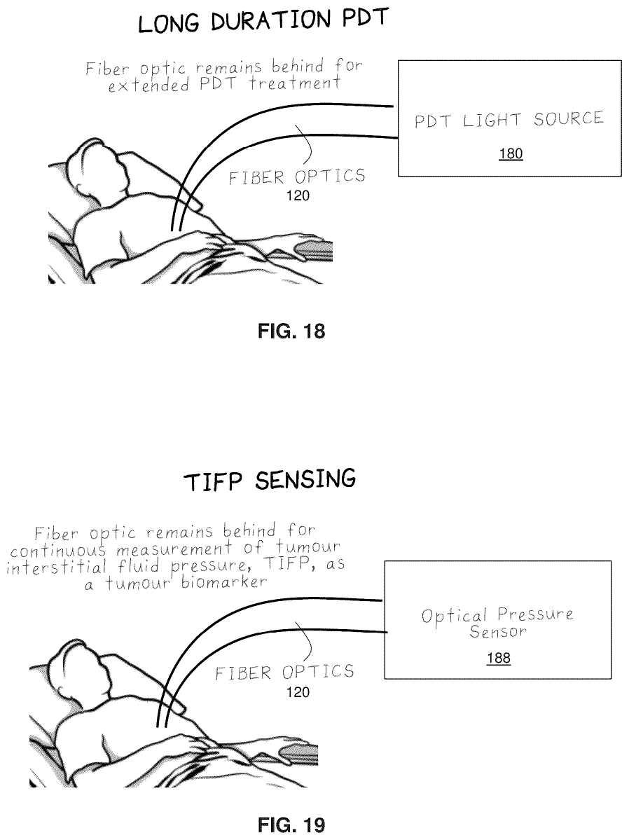

In another example embodiment, one or more optical fibers that were inserted into the subject may remain in place after the injection procedure, as illustrated in FIG. 18, for extended duration photo-dynamic therapy, PDT, via a PDT light source 180.

In another example embodiment, the optical fibers 120 can include an optical pressure sensor 188 for continuous measurement of IFP which can be a useful biomarker of the evolution of the tumor microenvironment and response to treatment, as illustrated in FIG. 19.

In another example embodiment, the system is configured so that the tumor tissue which is disrupted on contact with the laser energized fiber tip is purposely released into the tumor surroundings, by retraction of the needle or other means of flushing tissue from within the tumor or aspirating and reinjecting it outside the tumor, including into the vascular system so as to create an abscopal effect whereby the disrupted tissue contains tumor specific antigens that have not become damaged or denatured during disruption. This system can be deployed, for example, independently or in combination with drugs to activate the immune system, immune checkpoint inhibitors, T-cell, macrophage, dendritic cell, and innate immunity modulators.

Although the embodiments shown in FIGS. 8A-8C, 9A-B and 12 and 13, and described above, present two example embodiments of an intracorporeal laser pulse delivery assembly that facilitates both controlled delivery of PIRL laser pulses for tissue disruption and liquification, and controlled direct intracorporal injection of a pharmaceutical fluid (and optional aspiration), it will be understood that these example embodiments are not intended to be limiting, and that various aspects of the present disclosure may be implemented with alternative structural configurations of the intracorporeal laser pulse delivery assembly. For example, in some example implementations, the elongate conduit may include separate (e.g. parallel) lumen for the optical fiber and the fluidic channel. The multiple lumens may remain separate over the extent of the elongate conduit, emerging through separate distal apertures at the distal end of the elongate conduit, or may merge within the elongate conduit, for example, within a distal region of the elongate conduit. For example, FIGS. 15-17 show example implementations in which the optical fiber 120 resides within a first lumen 123 and the fluidic channel is defined within second lumen 124, and where the second lumen 124 is sealed or at least partially blocked when the optical fiber 120 is extended beyond a distal end of the second lumen 124.

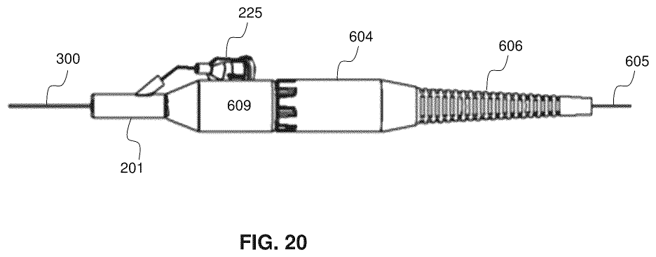

FIG. 20 shows a technical drawing of a prototype laser injector apparatus as shown in FIG. 12A, in which a distal portion including fluidics is separable from a fiber optic delivery cable for disposal or separate sterilization. The distal portion of the housing, shown at 609, combines the optical fiber (distal waveguide) and a fluidic sheath 300, which are sealed within a fluidic channel residing within a proximal elongated conduit 201, such that a sealed connection is made from the fluidic input port 225, and such that the fluid can be delivered close to the distal tip of the optical fiber. In this example implementation, the housing includes a distal portion 609 that is detachable from a proximal portion 604 that includes a means of inline connection between a proximal waveguide 605 (which is connected to or connectable to the PIRL laser source) and a short distal waveguide (e.g. optical fiber), which delivers the laser pulses on target. In this example implementation, strain relief is provided 606 for protection of the delivery cable or proximal portion of the waveguide.

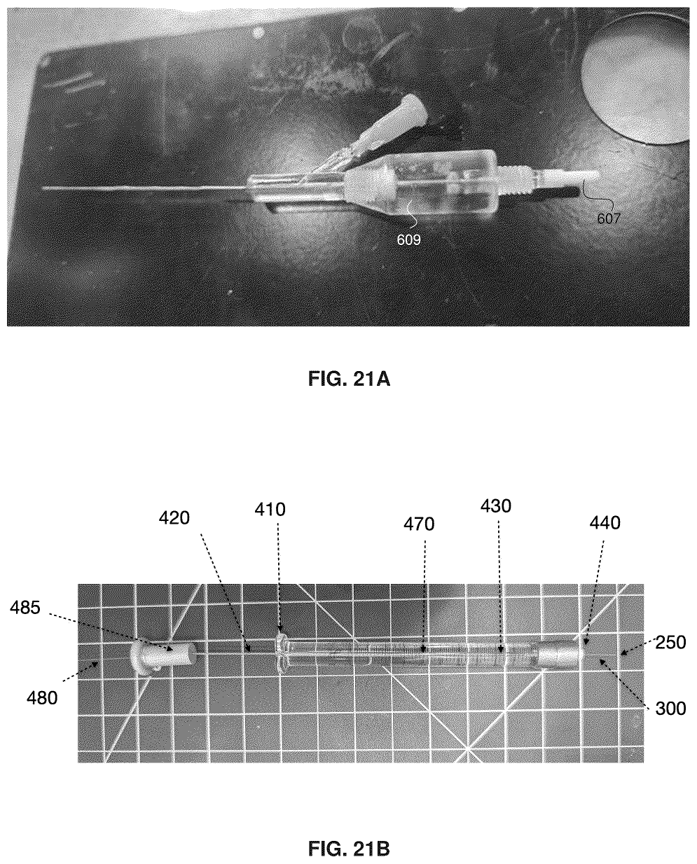

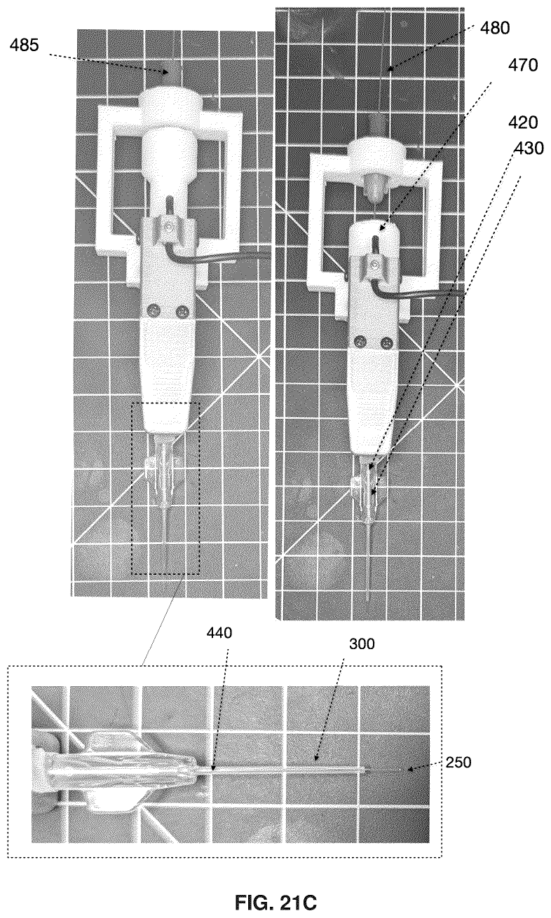

FIG. 21A shows a photograph of the example design of FIG. 20, adapted to include a distal removable portion that includes a fiber connector at the input side of the fiber optic for attachment to a fiber optic extension (patch cable) for delivery of laser energy to the distal section. The photograph shows the distal portion of the housing 609, of this separable prototype once disconnected, which includes a fiber optic ferrule 607 for connection of the distal portion of the waveguide to a longer fiber within the proximal portion of the housing (not shown).

FIG. 21B shows a prototype of an example embodiment of the injector design of FIG. 12B, created by modification of a standard Hamilton syringe. Here the solid plunger of the Hamilton syringe is replaced with a rigid hollow tube 420, which acts as a piston for the liquid. Inside the hollow lumen of the rigid body, the optical fiber 250 extends to the distal end and does not move relative to the syringe barrel 410. The hollow portion of the plunger contains a material that seals the fiber optic but allows motion of the piston relative to the static fiber optic. At the tip of the piston is another moving seal 470, which prevents escape of the fluid except to the distal end, while allowing the chamber volume to be changed by the position of the piston. On the proximal end of the plunger 420, a fiber connector 485 is embedded within the plunger handle for inline connection to an external optical fiber 480. The syringe barrel 410 contains the fluidic chamber 430, into which liquid can be drawn by retraction of the piston 420, and subsequently injected through the distal end of the chamber, where an elongated cannula 440 couples the fluidic channel into the fluidic sheath 300 that terminates near the distal tip of the fiber. The support housing 460, is not shown but could take the form of a motorized syringe pump.

FIG. 21C shows another prototype example embodiment of the injector design of FIG. 12B in which a motor is incorporated into the rigid housing 460, which controls the position of a piston inside the sealed fluidic chamber. In the left photograph, the chamber is volume is a maximum, i.e. the syringe is loaded, whereas in the right image, the piston has been repositioned by the motor until the chamber volume is minimum and the fluid from the chamber has been expelled through the distal tip.

FIG. 22 shows an example system embodiment in which the injector assembly 610 of FIG. 12A is incorporated into a motor 611 and is connected to a lead screw 612 to drive a linear stage 613 to control the position and speed of injection. The example system also incorporates a force sensor 614 to monitor the forces experienced by the distal tip at the distal end of the elongate distal portion 205, for use in a feedback mechanism that alters the control and position of the injector assembly relative to a fixed housing 615 to minimize tissue damage during injection. The system also shows the optical fiber 120 extending from the PIRL laser source 130 to the body/housing, and the fluid source 170 in fluid communication with the body/housing via fluidic channel 165. The example system this facilitates motorized motion for the piercing action and force sensor for monitoring of the frictional forces for optimal piercing speed and laser parameters.

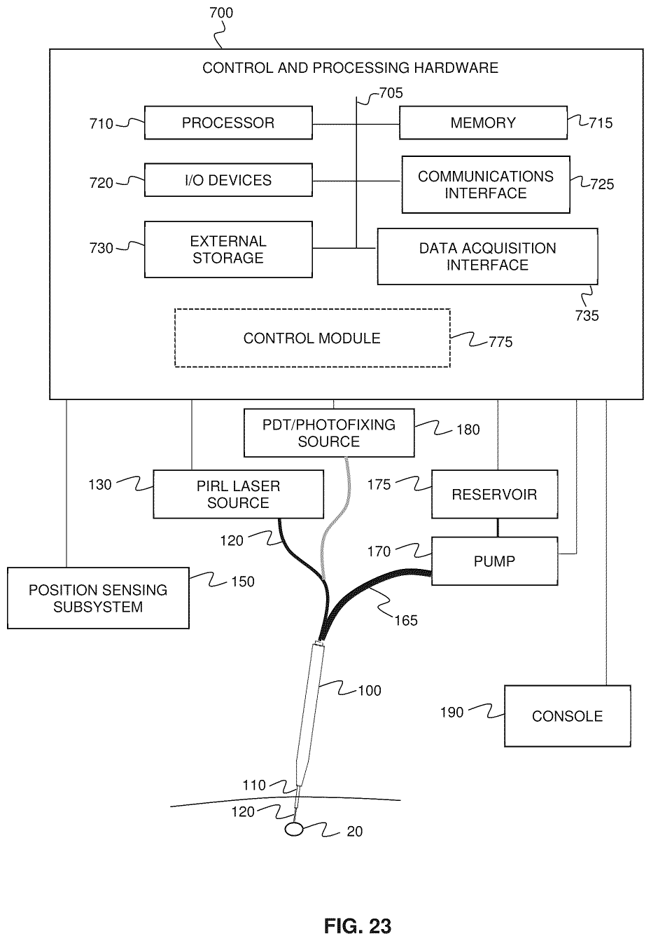

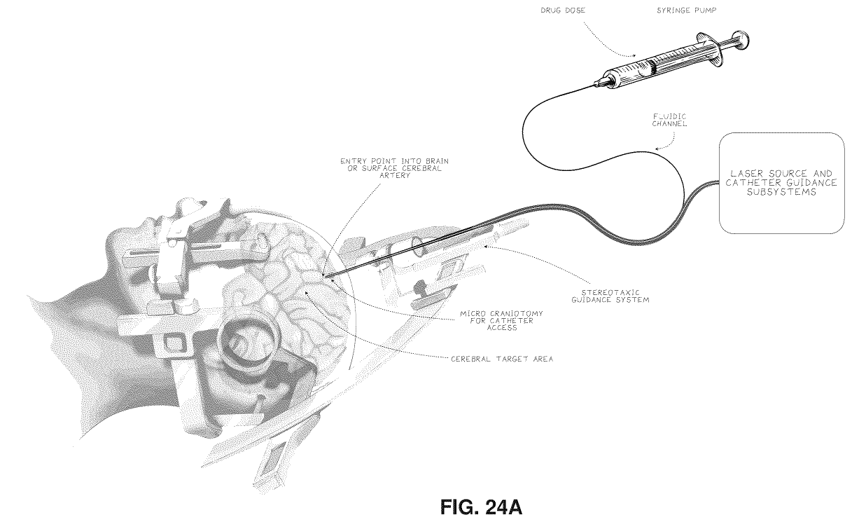

FIG. 23 illustrates an example system for performing PIRL-based local tissue disruption followed by direct local drug injection into the disrupted tissue. PIRL laser pulses are generated by a PIRL laser source 130, and the laser pulses are delivered through an optical fiber 120 that passes through a probe body 100 (handpiece) having a distal microcannula region 110, with the optical fiber 120 being optionally extendable from the distal end of the microcannula 110. The figure shows the insertable microcannula 110 being inserted into a subject with the distal portion of the optical fiber 120 extended to irradiate and disrupt a tissue region 20.

The laser source 130 is operatively coupled or connectable to control and processing hardware 700 for control thereof. The example control and processing hardware 700 may include a processor 710, a memory 715, a system bus 705, one or more input/output devices 720, and a plurality of optional additional devices such as communications interface 725, external storage 730, and a data acquisition interface 735. In one example implementation, a display (not shown) may be employed to provide a user interface for facilitating input to control the operation of the system 700. The display may be directly integrated into a control and processing device (for example, as an embedded display), or may be provided as an external device (for example, an external monitor).

A reservoir 175 contains a fluid therapeutic agent (e.g., pharmaceutical compound, drug), and delivery of the drug to the tissue region, through the probe body 100, is achieved by action of a pump 170 that is controlled by control and processing system 700.

Position sensing and guidance of the microcannula 110 and distal optical fiber 120 (or optical waveguide) is facilitated by position sensing subsystem 150, which is interfaced with the control and processing system 700.

The figure also shows the optional inclusion of an additional laser source that is also optically coupled (e.g., through a wavelength-multiplexing device or an optical coupler) to the optical fiber 120 or distal optical waveguide for the delivery of an additional form of optical radiation, such as a laser source suitable for photo fixing, photodynamic therapy, and/or photothermal disruption or thermally driven apoptosis.

The control and processing system 700 may include or be connectable to a console 190 that provides an interface for facilitating an operator to control the laser source 160. The console may include, for example, one or more input devices, such, but not limited to, a keypad, mouse, joystick, touchscreen, and may optionally include a display device.

The methods described herein, such as methods for controlling the sequence of operations of the local PIRL-based laser disruption and the subsequent fluid delivery, optionally controlling the extension and retraction of the optical fiber, and/or controlling one or more valves in fluid connection with the pump and/or reservoir, and other example methods described below, can be implemented via processor 710 and/or memory 715. As shown in FIG. 23, executable instructions represented as control module 750 are processed by control and processing hardware 700. Such executable instructions may be stored, for example, in the memory 715 and/or other internal storage.

The methods described herein can be partially implemented via hardware logic in processor 710 and partially using the instructions stored in memory 715. Some embodiments may be implemented using processor 710 without additional instructions stored in memory 715. Some embodiments are implemented using the instructions stored in memory 715 for execution by one or more microprocessors. Thus, the disclosure is not limited to a specific configuration of hardware and/or software.

It is to be understood that the example system shown in the figure is not intended to be limited to the components that may be employed in a given implementation. For example, the system may include one or more additional processors. Furthermore, one or more components of control and processing hardware 700 may be provided as an external component that is interfaced to a processing device. Furthermore, although the bus 705 is depicted as a single connection between all of the components, it will be appreciated that the bus 705 may represent one or more circuits, devices or communication channels which link two or more of the components. For example, the bus 705 may include a motherboard. The control and processing hardware 700 may include many more or less components than those shown.

Some aspects of the present disclosure can be embodied, at least in part, in software, which, when executed on a computing system, transforms an otherwise generic computing system into a specialty-purpose computing system that is capable of performing the methods disclosed herein, or variations thereof. That is, the techniques can be carried out in a computer system or other data processing system in response to its processor, such as a microprocessor, executing sequences of instructions contained in a memory, such as ROM, volatile RAM, non-volatile memory, cache, magnetic and optical disks, or a remote storage device. Further, the instructions can be downloaded into a computing device over a data network in a form of compiled and linked version. Alternatively, the logic to perform the processes as discussed above could be implemented in additional computer and/or machine-readable media, such as discrete hardware components as large-scale integrated circuits (LSI's), application-specific integrated circuits (ASIC's), or firmware such as electrically erasable programmable read-only memory (EEPROM's) and field-programmable gate arrays (FPGAs).

A computer readable storage medium can be used to store software and data which when executed by a data processing system causes the system to perform various methods. The executable software and data may be stored in various places including for example ROM, volatile RAM, nonvolatile memory and/or cache. Portions of this software and/or data may be stored in any one of these storage devices. As used herein, the phrases “computer readable material” and “computer readable storage medium” refers to all computer-readable media, except for a transitory propagating signal per se.

The example embodiments described above can be employed for a wide variety of clinical applications. It will be understood that the aforementioned example therapeutic applications involving direct intratumoral injection and local chemotherapy are but one example implementation and are not intended to limit the scope of the present disclosure. It will be understood that the example embodiments may be employed for a wide variety of other applications, including, for example, the local delivery of fluids to tissues other than cancerous tissue, for example, for site selective drug treatments or microbiopsies for detection of cancer or other disease states.

While many of the example embodiments disclosed herein pertain to the local delivery of therapy to tumor tissue, it will be understood that the present example embodiments are not intended to be limited to the local treatment of tumor tissue and can alternatively be employed to delivery local therapy to any intracorporeal tissue region, or to provide internal access to any intracorporeal tissue for any minimally invasive treatment modality. For example, the present example methods may be employed to treat a wide range of pathologies associated with internal tissue, such as, but not limited to, localized disruption ablation of cardiac tissue to arrest fibrillation, and onset of heart attacks, removal of tissue around pinch nerves involved in chronic pain without the complication of scar tissue formation to reinjure nerves and reoccurrence of chronic pain, intravascular surgery, repair of vessels involved in internal bleeding such as strokes by thermal coagulation using the same form of beam delivery, removal of nasal and vocal cord polyps, creating openings for improved blood circulation, implants requiring the formation of cavity within tissue such as micro-cochlea implants, benign lesions, autoimmune diseases affecting specific, cardiovascular diseases such as atherosclerosis characterized by arterial fatty deposits, fibrotic diseases including pulmonary fibrosis and liver cirrhosis, and restenosis, where excessive tissue growth occurs after procedures like angioplasty, and nerve tissue disruption and orthopedic procedures.

In some example implementations, the example embodiments disclosed herein may be employed for intracranial applications including, but not limited to, local chemotherapy of brain metastases, neuromodulation and neurostimulation. For example, the advancement of a PIRL-based probe (e.g., an optical fiber or probe housing an optical fiber or other optical waveguide for delivery of PIRL pulses), via PIRL-based tissue disruption and liquification with minimal collateral damage along the entry path, may be employed to enter the brain with minimal collateral damage to surrounding tissue. Such a minimally invasive PIRL-based probe may be employed for the treatment of intracranial pathologies, e.g., via tissue disruption and liquification and/or thermal treatment. A probe configured to facilitate both PIRL tissue disruption and liquification and local fluid delivery, such as the example embodiments disclosed above or variations thereof, may be employed to facilitate atraumatic entry into the brain with the subsequent local delivery of therapeutic agents within the brain, optionally with PIRL-based tissue disruption and liquification and/or thermal treatment of internal tissues. Here the ability to enter the brain without tissue deformation, deflection from the optimal path to the region of interest, or collateral damage is a critical for the absolute minimally invasive procedure for brain tumors.

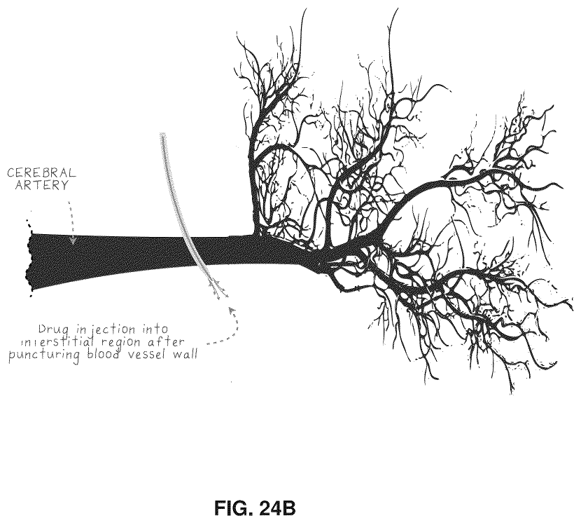

FIG. 24A illustrates a system and method of accessing the cranial vasculature, where imaging and stereotaxic guidance is used to navigate a catheter into a specific surface cerebral arteries without travelling through a long section of the vasculature. This is accomplished by creating an entry point in the skull in close proximity to an entry point in a cerebral artery close to the surface of the brain. Accordingly, in some example implementations, the example systems described herein may be employed for direct intracranial injection of a pharmaceutical fluid, without relying on intravascular navigation. Accordingly, in some example embodiments, the intracorporeal target site resides within a brain of a subject, and the elongate distal portion of the intracorporeal laser pulse delivery assembly is a microcannula suitable for incorporeal insertion, and the pulsed infrared laser source is controlled to perform local tissue disruption and liquification to facilitate insertion of the microcannula through a skull of a subject, and the positioning of the distal end of the microcannula within the brain of the subject for direct injection of a pharmaceutical fluid.

As shown in FIG. 24B, however, in some cases, when employing a PIRL-based intracorporeal laser pulse delivery assembly having a microcannula configured for delivery of PIRL laser pulses and delivery of a pharmaceutical to an intracranial target region may be interrupted by the presence of an artery, such as a cerebral artery. In such cases, as shown in the figure, during a microPIRL-based tissue disruption and liquification can be employed to facilitate entry through the vessel wall. Moreover, as described below, additional laser pulses may be delivery to facilitate coagulation and repair of the vessel wall after withdrawal of the microcannula.

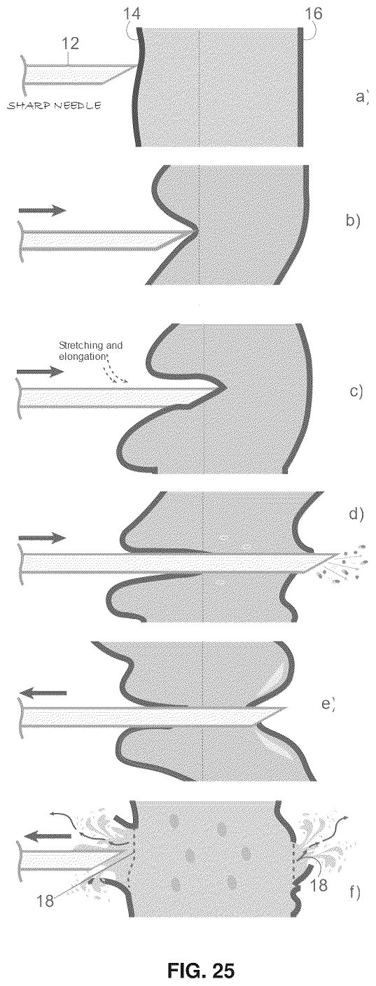

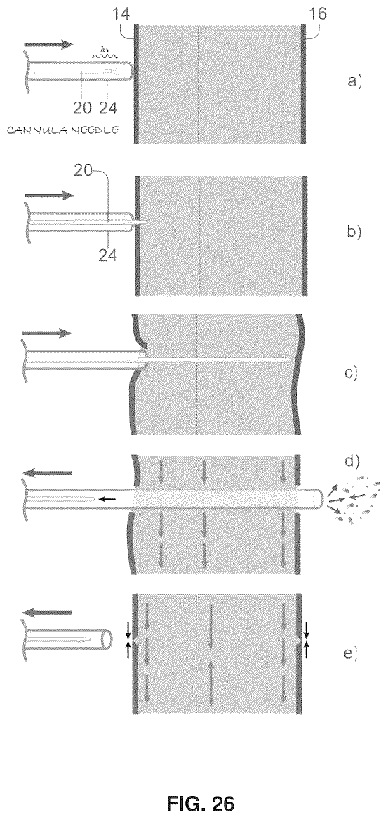

This approach can be compared to the conventional methods of vascular puncture that are unassisted by PIRL laser pulses. At present, the usual method of delivering fluids involves piercing a vessel with a needle and injecting the drugs, usually in liquid form. FIG. 25, in steps a) to f) (top to bottom in the figure), illustrate this sequence of events when a needle is penetrating an anatomical vessel. In step a), contact is made between the sharp tip of the needle 12 and the outer wall 14 of the blood vessel, step b) the vessel wall is deformed as the needle penetrates through the vessel wall and the tissue of the vessel wall sticks to needle by sheer forces. Step c) shows the vessel wall undergoing elongation/stretching/deforming as the needle 10 penetrates through the vessel and again the vessel tissue sticks to needle by sheer forces. Step d) shows the needle piercing the other vessel wall 16 with the same deformation and shows fluid being injected into the adjacent section of tissue, which would be the patient's brain if such an approach was used. Step e) shows the needle 10 being retracted with the deformed vessel sticking to needle. Step f) shows needle 10 fully withdrawn and outside the vessel after drug delivery into brain past the blood-brain barrier but now major bleeding is seen out of both opening/perforations in the vessel. This schematic representation of everyday experience with needle delivery would create severe damage especially within the fragile structure of the brain and its complex network of neurons. Thus, this figure illustrates how current methods of piercing the blood-brain barrier is problematic and would lead to bleeding and damage to the brain.

A means to introduce drugs of arbitrary mass into the interstitial fluid of the brain would allow cures for numerous diseases and allow completely new drug regimes for all classes of mental illness. Furthermore, the cause-and-effect relationships of mental diseases could be determined much better by being able to introduce controlled doses with well defined distribution patterns in the brain. The problem is introducing drugs past the blood-brain barrier. It serves an essential role in protecting the brain from viral and bacterial infection, however, it has made the brain susceptible to anomalies without means to reset brain function or to help control flares in neural firings as in epilepsy.

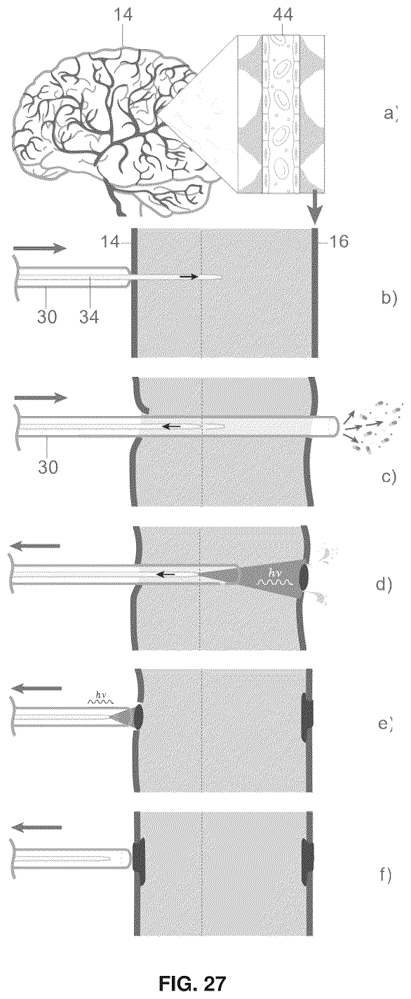

Referring to FIG. 26, steps a) to e) (top to bottom in the figure), illustrate one example embodiment of the present disclosure involving the creation of a small opening on the order of a diameter of the distal end of the microcannula so small that there is no, or minimal, leakage or damage to tissue in the region used for the drug delivery. The procedure shown in step a) involves guiding an optical fiber 20 contained in a cannula needle 24 to a blood vessel (e.g., jugular vein, carotid artery, etc.), sinus passage or other natural passage. The optical fiber 20 is coupled to a laser source and is used to deliver laser pulses to the vessel wall 14. The optical fiber 20 is about 200 microns maximum in diameter and can be tapered down to a tip 26 in the 10 micron range to further decrease the diameter of the hole for drug delivery. The fiber beam delivery as described allows the focusing of the laser beam delivered by fiber 20 to energize the tissue material sufficiently to disrupt the tissue and create a hole on the order of the diameter of the fiber tip 26. For 10 to 30 micron tips, the size of the hole should be on the order of a single cell. In other words, an important point is that the system produces a very small hole so water/interstitial fluid surface tension is too strong to allow leakage through the walls 14 and 16 once the fiber 20 and cannula needle 24 perform the operation of creating a pathway through the vessel (which may or may not include a blood-brain barrier) or just slow leakage/weeping that can be sealed, for example, according to the method illustrated in FIG. 27.

For sufficient infrared laser light transmission and required hardness, the fiber 20 will typically be sapphire, but could also be diamond, SiN, or fused quartz for applications not using Infrared laser wavelengths or using metal coated tips of the same materials for creating highly localized heating. The fiber as defined above with tapered tip 20 must be sufficiently hard to withstand the forces in disrupting tissue. The fiber 20 must have a minimum hardness of about 8 GPa and compressive strength of 1 GPa to avoid fracture of the tip due to the thermally created stress in superheating the water/tissue region.

The opening is created by selectively exciting vibrational modes in the infrared but other mechanisms of light absorption can be used such as exciting heme transitions in oxygen transport heme proteins or other strongly absorbing transition for light absorption that leads to conversion to heat. Similarly, a metal coating or other material such as an oxide or chemically bonded molecular layer on the above fiber structure that strongly absorbs light and converts rapidly to heat faster than thermal diffusion away from the heated area, shown in FIG. 27. The transduction of light to heat in this process occurs in the impulsive limit to ensure uniform forces leading to tissue disruption and entry of the fiber 20 into the desired interstitial space. The ideal embodiment involves the selective excitation of water through its very strong absorption in the infrared region of the light spectrum, as a universal absorber in all cell types or tissue. The extremely strong infrared absorption is due to the large dipole moment of water associated with its vibrational stretching modes, with absorption occurring over depths corresponding to the dimensions of single cells. The selective excitation of water molecules in the cells creates superheating conditions that lead to homogeneous nucleation whereby all the nucleation sites (bubbles of gas typically but liquid domains also apply) are uniformly distributed and create uniform forces. The rapid thermal expansion and uniform forces lead to cell rupture and effectively convert the tissue to a liquified medium much like a protein shake.

The same can be done with other absorption bands at the expense of requiring more pulse energy and involved deeper penetration depths for the light absorption. The laser pulse duration for all modalities, including other means such as electrical resistive heating, must be shorter than the time for thermal diffusion over the dimensions of the excited or energized volume. For the case of 200 micron fiber tips 26, this time based on typical thermal diffusion constants close to that of water corresponds to 100 millisecond timescales in the lateral direction. However, most of the heat diffuses away in the longitudinal direction along the normal to the fiber tip 26 as the absorption depth of order 10 microns is much shorter than the diameter of the optical fiber 20 for use of a 100 micron scale fiber delivery systems. This time scale corresponds to thermal diffusion times of order 100 microseconds in the longitudinal direction.

In the case of selectively exciting water in tissue, laser wavelengths of approximately 3 microns, tuned to the OH vibrational stretch frequency are used which has an absorption depth of about 3 to about 10 microns for typical water densities. The thermal diffusion time along this direction leads to a decay in the thermal profile on the 10-100 microsecond time scales. The pulse should be shorter than 100 microseconds. The other limiting time constraint for the pulse profile is the thermal expansion and loss of energy confinement to drive homogenous nucleation. In this case, the pulse should be shorter than about 10 ns. The ultimate limit is defined by the time scale for nucleation growth which depends on the tissue with respect to the barriers to creating gas or liquid void spaces. It is key to have pulses shorter than the time scale for unarrested nucleation growth that leads to large bubbles, cavitation, and shock wave generation with bubble collapse and focusing of all the stored energy into shear waves. The upper limit in pulse duration is about 100 microseconds based on thermal diffusion times and optical penetration depths. An ideal embodiment is to use a Pulsed InfraRed Laser (PIRL) with pulses <1 ns to selectively excite water in the impulsive limit for full energy confinement and completely homogeneous phase transitions of the tissue from solid to liquid to gas for the finest tissue disruption without any energy transport or possible damage to adjacent tissue. This time scale is sufficiently short to ensure nucleation growth for all tissue types is homogeneous. The system and method of the present disclosure is not limited to infrared as other absorption bands can be used but adhering to the same limits with respect to the creation of uniform nucleation and tissue disruption to create a hole with minimal collateral damage to surrounding tissue.

In all cases of laser wavelength, pulse duration, fiber diameters respecting the above physical constraints, the fiber end tip 26 must be of a material sufficiently hard to withstand the very large forces generated in disrupting the tissue. As noted above, for sufficient infrared light transmission and required hardness, the tip 26 will typically be sapphire, but could also be diamond, SiN, or fused quartz for applications not using Infrared laser wavelengths.

The action of the PIRL in the above limit is to create a hole or pathway for injection of drugs without any collateral damage to adjacent tissue. The hole is small enough that the typical surface tension of blood, plasma, interstitial fluid, water is too high to allow leakage out of the small orifice. The effect is much like the physics involved in breathable rain resistant coatings used for rain gear. In the creating of the hole, the optical fiber is advanced as the tissue disruption occurs over 3-10 microns, with approximately the same depth as the absorption depth. The laser repetition rate is controlled to allow the optical fiber 20 to advance into the vessel wall 14, as shown in FIG. 26, step b), without accumulating heat in the same location that could lead to thermal damage, trigger apoptosis of surrounding tissue and necrosis. The typical tissue disruption depths for blood vessels are millimeter to submillimeter such that the opening takes on the order of just 1000 laser shots or a few second to create depending on the laser repetition rate. No leakage will occur during this process to create an opening for drug delivery.

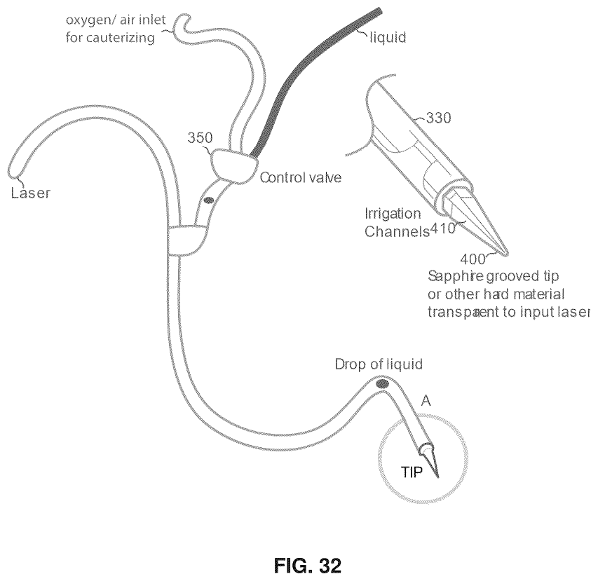

FIG. 26, step c) shows the cannula needle 20 having penetrated through the other side 16 of the vessel once an opening has been produced by the laser beam. Once totally through the vessel the fiber and associated tip 26 is withdrawn backwards to create a space for the fluid to be injected into the tissue adjacent to side wall 16. The drug/liquid is injected after by way of a valve with a T connection, see FIG. 32, after the hole in vessel wall 16 is created and vessel (and, if the vessel is sufficiently small, the blood-brain barrier) has been crossed. FIG. 26, step d) shows the cannula needle 20 and fiber 20 and tip 26 fully withdrawn from the vessel. FIG. 26, step e) shows the holes in sections 14 and 16 undergoing sealing.

The optical fiber 20 may be housed within the finest bore cannula needle 24 to be used for drug delivery while keeping the overall diameter of entry with the optical fiber 20/needle 24 as small as possible. The cross section can be made as small as 200 microns for hollow fiber which can be used for both guiding the laser to the zone to be cut as well as subsequent steps involving drug delivery. The optical fiber in contact mode will make the hole in the vessel or entry point by extending the fiber out of the needle to the extent needed. This motion is typically about 1 to about 10 mm. Once the opening is created, the optical fiber 24 is retracted within the needle 20, with the needle 20 positioned at the surface of the opening with sufficient, but minimal, pressure to make a seal. The drug is then injected in the interstitial fluid, spinal fluid, or other suitable location for optimal delivery of the drug for a particular treatment regime.

FIG. 27, steps a) to f) shows essentially the same process as shown in FIG. 26 but with specific emphasis on penetrating a blood vessel normal to the surface of brain tissue through a cranial vessel (which may include the blood-brain barrier) in order to deliver drugs to the patient's brain. In this figure, the disclosure covers the possible occurrence of blood seepage, which within the brain is highly detrimental. The occurrent of blood leakage can be mitigated and as an additional safety measure by including another laser to selective coagulate the blood exactly at the aperture or leakage site. Steps shown in FIG. 27, steps d) and e) shows that closing the holes after removal may preferably be done with green laser light, as the liquid will not transmit IR, with the green laser inducing coagulation over a larger volume than the IR would and provides enough coagulated protein or “putty” to act as patch to seal off any leakage.

The optical fiber 20 at the position of opening is used to close the hole completely to fully prevent blood or other fluid leakage by cumulative heating to locally coagulate blood or other proteinaceous matter to seal the vessel. Air may be injected through the needle 20 to locally cauterize, burn tissue, for a hard seal to prevent any occurrence of leakage at the opening.

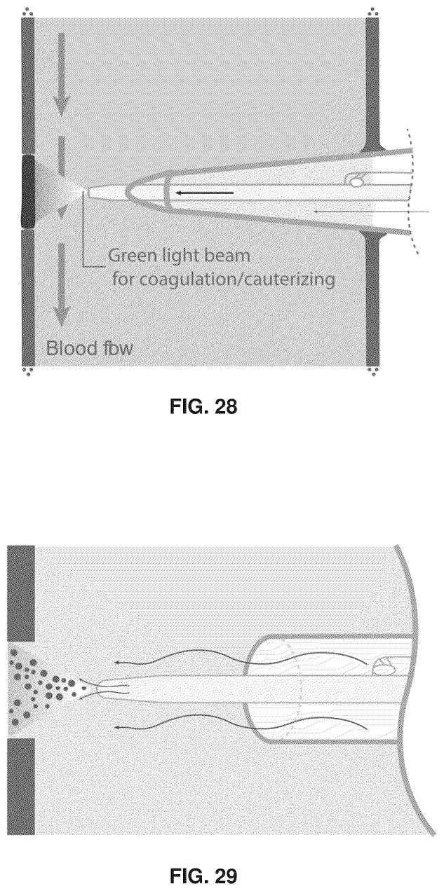

FIG. 28 illustrates using a green light beam upon withdrawal of the needle selectively excite blood thereby inducing coagulation and/or cauterization to stop the bleeding during retraction of the needle once the vessel has been penetrated and fluids delivered.

FIG. 29 shows the cannula or needle injecting drug towards thermally expanding blood/liquid flow and bubble stream, (solid circles) which keeps the aperture past the vessel (and, if the vessel is sufficiently small, the blood-brain barrier layer) open. The micro to nanobubbles from the PIRL output creates a highly directed stream to keep the aperture in the vessel open and directs the injected drug past the vessel. In the case where it is not possible to have the needle make a seal to the vessel wall where the aperture is located, the fiber once it has created the aperture in the vessel (and optionally the blood-brain barrier if the vessel is sufficiently small), then is withdrawn a small distance from the aperture. Here rather than sealing the vessel, there is a continuous stream of laser induced impulsive heating and resulting bubbles that keeps the aperture open. The needle can then inject the drug and the bubble plus thermal expanding plume directs the drug to the opening in the vessel. It is the bubbles that keep the vessel (and optionally a blood-brain barrier layer within the vessel) open to allow drug infusion from a remote site from the aperture. Ultrasound is currently used with nanobubbles to microbubble and an acoustic resonant cavity around the head to jack hammer the blood-brain barrier to allow drug diffusion.

The subsequent drug injection will allow the drug to diffuse within the brain as the hole created above will be deliberately be made to go past all the closely knit endothelial cells and dense perineurium and, if the vessel is sufficiently small, associated layers that constitute the blood-brain barrier. The drug diffusion will be constrained by the free volume defined by the brain vasculature to the same extent small molecules diffuse within the brain such as glucose, O₂, etc. for brain function. With characterization of the diffusion for a particular drug, molecule, or biological molecule, the drug delivery for a desired purpose can be made quantitative in terms of drug dose per location in the brain. This direct injection procedure solves the blood-brain barrier problem in limiting the use of drug treatment regimes involving molecules larger than 400 Da.

In the case that there is no suitable natural pathway to allow drug delivery or a very specific region in the brain is required for a particular treatment, without the involvement of other regions of the brain, the present disclosure also allows for localized drug delivery. This latter feature is specifically documented here for the treatment of brain tumours but may also be of great value for the treatment of epilepsy, chronic pain, drug addiction, or other diseases of the brain or disruptive regions of collective brain function. Mapping different brain functions using MRI is allowing epicentres for various mental illnesses to be identified that will allow for effective drug delivery with the minimal dose for optimal efficacy. Here the procedure involves the creation of 1 mm to submm hole in the cranium using line of sight laser ablation with a PIRL system. In this case PIRL action leads to ablation of skin, bone, lining, and intervening tissue by which the thermally deposited energy is transduced into translational motion with effectively no lateral thermal or shock wave damage or tissue disruption. There is perfect removal of tissue with single cell level of accuracy. This extremely small hole, as above, is small enough to avoid significant blood or fluid loss. This hole is created at the position desired for surgical access to the desired brain location with optimal pathway. The above procedure is repeated now as above using fiber beam delivery through the hole created in the cranium to enable creation of a pathway to the desired target location to give quantitative drug delivery selective to a specific region in the brain.

In summary, the present disclosure provides a method for direct intracranial drug delivery. The method uses an optical fiber to guide laser light inside the brain for direct interstital drug delivery. The diameter of the fiber tip is sufficiently small to create a hole which is too small to allow significant leakage of fluid (spinal, interstitial fluid, blood). The laser radiation of the laser guide is specifically tuned to selectively excite water in the 2 to 20 micron or other strongly absorbing spectral region to deposit energy within the diameter of the fiber tip with an absorption depth on the order of about 10 to about 100 microns, or a few cell diameters, to deposit the least amount of energy possible to drive tissue disruption. Other strongly absorbing wavelength bands in the infrared part of the light spectrum include amide stretch excitation around 4-6 microns, and similarly strong vibrational modes in the 10 micron range for a number of vibrational modes of the constituent proteins/lipids that form the vessel walls. The same concept can be used in exploiting strong absorption by other absorbers in tissue to strongly localize the absorbed energy and use the minimal energy to drive tissue disruption to create the hole.

Alternatively, universal excitation of tissue can be made readily possible, equivalent to selectively exciting water, by using UV absorption in 200-300 nm range where all amino acids absorb light to create excited states and subsequent nonradiative relaxation channels into heat. The use of 6 micron IR excitation may be used to excite tissue for heat deposition, for example, collagen or any other structural material within the blood vessel or tightly knit endothelial cells (ECs), pericytes (PCs), capillary basement membrane, and astrocytes which may have relatively low water content compared to blood vasculature elsewhere in the body. The basic concept of the present disclosure is to exploit the strongest absorption band possible to localize the light absorption to the vessel walls to use the minimum energy possible to produce homogeneous, uniform nucleation to drive phase transitions to locally disrupt tissue in the volume defined by the absorption of light.

This condition eliminates unarrested or exponential nucleation growth, cavitation, and shock wave generation that would lead to collateral damage and inelastic deformation of the vessel wall that would lead to bleeding. The concept involves making a hole in the vessel with no collateral damage, all tissue defining the aperture are still fully elastic and keep the hole conserved with diameters too small to allow leakage. The hole being small enough that the surface tension of the blood or other bodily fluid prevents flow out of the vessel. The elasticity of the tissue in most cases will close upon exit of the fiber from the vessel much like a septum for a needle. In this case, the delicate nature of the vessel is easily deformed by a needle and excessively bleed. By creating the hole in the vessel with no shear forces, the vessel walls stay perfectly elastic without deformation and thereby close upon exit of the fiber/cannula assembly. In certain cases, the vessel may be deliberately closed upon exit by using a laser to selectively excite the blood at the opening to coagulate the blood and seal the hole. This additional feature adds a margin of safety to ensure minimal bleeding into very sensitive brain tissue without any change in interstitial fluid pressure due to leakage to cause trauma.

The present example embodiment is not intended to be exclusive to IR transitions but covers any means to localize energy, convert to heat, to drive tissue disruption faster than exponential nucleation, cavitation, and shock wave generation. In particular, the use of UV laser radiation would follow the same prescription as the use of IR for selective excitation of water. The absorption of UV is as universal as the absorption of IR by the vibrational modes of water and the number density of chromophores are comparable in that all structural material, proteins, and other building blocks absorb in the UV. This wavelength range has unwanted photochemistry as a side product and would only be used for low water content tissue.

One example embodiment involves the exploitation of IR wavelengths tuned to selective excite water in the tissue as this absorption band is the strongest absorption and couples the absorbed light energy completely to translation, rotational motions, or heat, to impulsively excite the tissue matrix well above the critical temperatures for undergoing phase transitions. The laser action acts to strongly drive phase transitions from solid to liquid to gas fast enough to ensure any nucleation (liquid domains, gas bubbles) are uniformly distributed. The laser pulse duration is short enough to provide rapid, homogeneous, nucleation, with the nucleation diameters small enough to avoid unarrested or exponential nucleation growth as a few locations that lead to large (>10 micron) domains or bubbles that undergo cavitation and shockwaves that otherwise lead to massive collateral damage. The action of the PIRL fiber guided drug delivery system disrupts tissue to allow a liquid flow like access (“hot knife in butter”) to desired regions without tissue deformation.

The path to the desired entry point can be made in any direction desired without risk of deflection or misguided operation, and further the whole procedure produces little or no collateral damage. The latter point is a key point in the disclosure in that the uniform tissue disruption removes tissue heterogeneity in advancing the fiber to the target location without tissue deformation or risk being deflected from the desired target tissue. The extremely small hole will seal upon exit with no bleeding, to avoid the associated known risks for damaging brain tissue. The vessel can also be deliberately sealed as an additional safety measure upon exit, by deliberately accumulating heat to lead to coagulation or as desired cauterization to close the entry point. This procedure allows full access to the interstitial regions of the brain with no trauma to either the entry point for drug delivery or the access channel created. The tissue displacement to allow fiber passage to the target site for drug injection is done within the elastic limit of the tissue with no collateral damage to the tissue in creating the passage. Upon exit of the fiber the wound closes without any residual trauma. Only the needle will create damage as per normal use of needles and this range of motion is limited to regions where the needle access to the vessel of interest does not cause loss of function. This limited level of damage can be further eliminated entirely by the use of hollow core fiber delivery systems for both transporting the laser light (hollow core fiber+sapphire tip or other hard optically transparent material) and the drug to the target site. The hole created can be made anywhere in the brain with the above prescription for quantitative drug delivery of any arbitrary mass molecule, biological, or functionalized material that can be mechanically transported by fluid injection through a needle or hollow core fiber optic.

Thus, the above disclosed process solves the problem of quantitative drug delivery of any drug, functionalized material, or biological substance into the brain by creating a passage through a vessel, and in some cases, the blood-brain barrier, without trauma or collateral damage to the tissue involved.

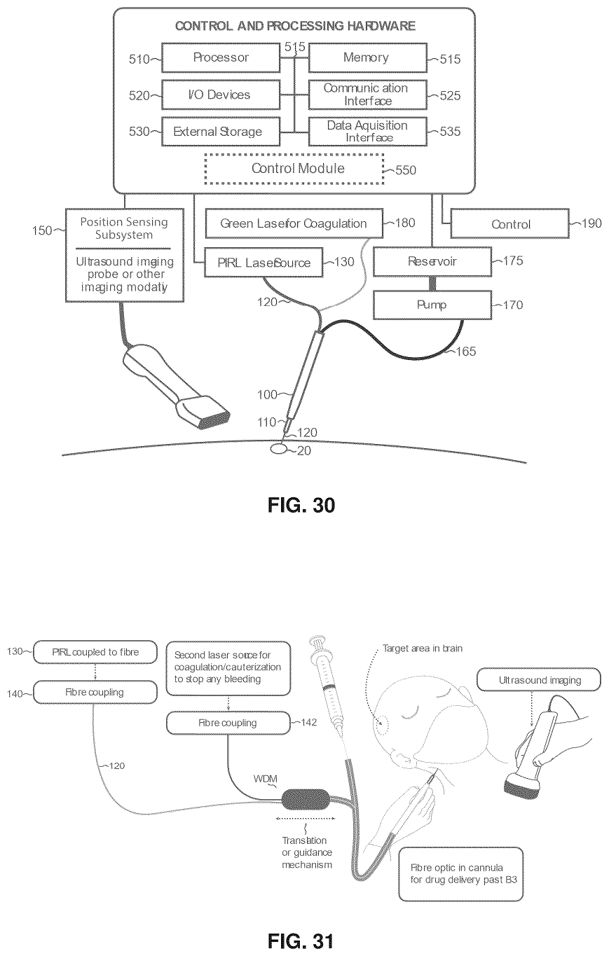

FIG. 30 illustrates an example system for performing PIRL-based direct intracranial drug injection using a laser pulse delivery tool having a distal optical waveguide for beam delivery and an integrated fluidic channel for targeted drug delivery. PIRL laser pulses are generated by a PIRL laser source 130, and the laser pulses are delivered through an optical fiber 120 that passes through a probe body 100 (handpiece) having a distal microcannula region 110, with the optical fiber 120 being optionally extendable from the distal end of the microcannula 110. The figure shows the insertable microcannula 110 being inserted into a subject with the distal portion of the optical fiber 120 extended to irradiate and penetrate a tissue region 20.

The laser source 130 is operatively coupled or connectable to control and processing hardware 500 for control thereof. The example control and processing hardware 500 may include a processor 510, a memory 515, a system bus 505, one or more input/output devices 520, and a plurality of optional additional devices such as communications interface 525, external storage 530, and a data acquisition interface 535. In one example implementation, a display (not shown) may be employed to provide a user interface for facilitating input to control the operation of the system 500. The display may be directly integrated into a control and processing device (for example, as an embedded display), or may be provided as an external device (for example, an external monitor).

A reservoir 175 contains a fluid therapeutic agent (e.g., pharmaceutical compound, drug), and delivery of the drug to the tissue region, through the probe body 100, is achieved by action of a pump 170 that is controlled by control and processing system 500.

Position sensing and guidance of the microcannula 110 and distal optical fiber 120 (or optical waveguide) is facilitated by position sensing subsystem 150, which is interfaced with the control and processing system 500. The figure also shows the optional inclusion of an additional laser source that is also optically coupled (e.g., through a wavelength-multiplexing device or an optical coupler) to the optical fiber 120 or distal optical waveguide for the delivery of an additional form of optical radiation, such as a laser source suitable for photo fixing, photodynamic therapy, and/or photothermal disruption or thermally driven apoptosis.

The control and processing system 500 may include or be connectable to a console 190 that provides an interface for facilitating an operator to control the laser source 160. The console may include, for example, one or more input devices, such, but not limited to, a keypad, mouse, joystick, touchscreen, and may optionally include a display device.

The methods described herein, such as methods for controlling the sequence of operations of the local PIRL-based laser penetration and the subsequent fluid delivery, optionally controlling the extension and retraction of the optical fiber, and/or controlling one or more valves in fluid connection with the pump and/or reservoir, and other example methods described below, can be implemented via processor 510 and/or memory 515. As shown in FIG. 30, executable instructions represented as control module 550 are processed by control and processing hardware 500. Such executable instructions may be stored, for example, in the memory 515 and/or other internal storage.

The methods described herein can be partially implemented via hardware logic in processor 510 and partially using the instructions stored in memory 515. Some embodiments may be implemented using processor 510 without additional instructions stored in memory 515. Some embodiments are implemented using the instructions stored in memory 515 for execution by one or more microprocessors. Thus, the disclosure is not limited to a specific configuration of hardware and/or software.

It is to be understood that the example system shown in FIG. 30 is not intended to be limited to the components that may be employed in a given implementation. For example, the system may include one or more additional processors. Furthermore, one or more components of control and processing hardware 500 may be provided as an external component that is interfaced to a processing device. Furthermore, although the bus 505 is depicted as a single connection between all of the components, it will be appreciated that the bus 505 may represent one or more circuits, devices or communication channels which link two or more of the components. For example, the bus 505 may include a motherboard. The control and processing hardware 500 may include many more or less components than those shown.

FIG. 31 shows a patient undergoing direct intracranial drug delivery with a PIRL-based intracranial injection system and includes a translation or guidance mechanism for assisting the clinician in placing the tip.

In some example embodiments, in which fluidics are coupled to a hollow fiber which share the hollow channels to the tip, gas and/or liquid can be injected into the waveguide channel to deliver liquid and then empty the channel for transmission of laser pulses, as shown in FIG. 32. Specifically, FIG. 32 illustrates an example configuration in which a sapphire end piece 400 is provided at a distal region of a hollow waveguide 330, the sapphire end piece including one or more irrigation channels 410 is in fluid communication with a central lumen of the hollow waveguide. The figure illustrates an example implementation in which a plurality of irrigation channels is located on the sides of the taper. A compact and minimally perturbative fiber delivery solution may be achieved by employing a photonic crystal or hollow fiber. The end piece, which is preferably sapphire, is IR transparent and sufficiently hard to withstand the laser driven shock waves involved in the tissue disruption subsequent to impulsive infrared pulse heating.

Additional fluidic channels can be integrated with additional control valves for liquid or gas aspiration, irrigation, to control the pressure within the channel and for priming of the channel with liquid. As shown in FIG. 32, a hollow fiber waveguide may be employed to guide the PIRL pulses for tissue disruption to create the drug delivery pathway. The hollow fiber may be equipped with a tapered sapphire tip 400 that includes one or more irrigation channels 410. A control valve 350 allows selection between delivery of liquids (drugs etc.) and oxygen or air for cauterization.

In some example embodiments, systems and methods are provided that facilitate the delivery of a pharmaceutical fluid to an intracranial target, in which the distal portion of the elongate conduit is configured as a microcatheter that can be employed to navigate the vasculature system and employ the delivery of PIRL pulses to access the target, beyond the blood-brain barrier.

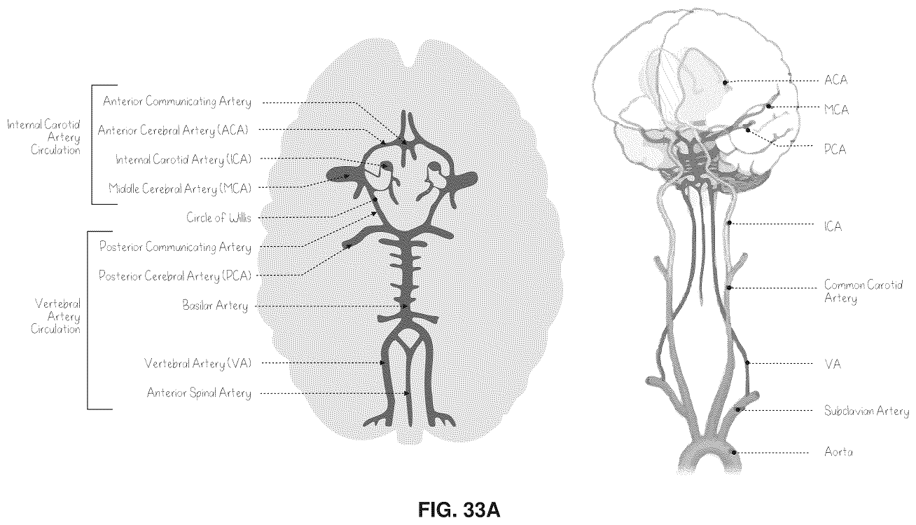

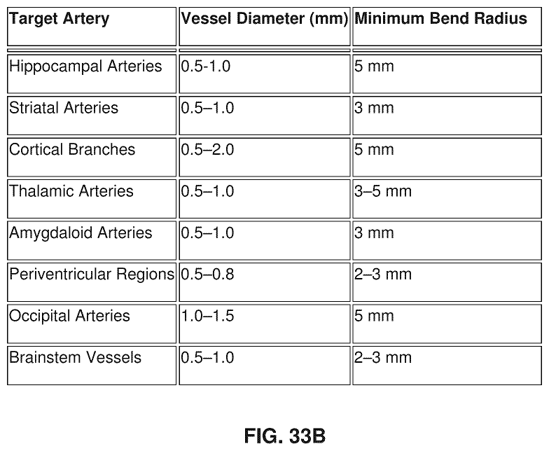

FIG. 33A shows schematic maps of the vascular anatomy of the brain. Vascular targets for clinical treatment may include, for example:
a. Hippocampal Arteries: Accessing the hippocampus to deliver treatments for neurodegenerative conditions like Alzheimer's disease.
b. Striatal Arteries: Targeting the basal ganglia for Parkinson's disease therapies.
c. Cortical Branches: Reaching various cortical areas to treat brain tumors or cortical lesions.
d. Thalamic Arteries: Managing chronic pain or epilepsy by targeting the thalamus.
e. Amygdaloid Arteries: Addressing anxiety disorders or PTSD by delivering agents to the amygdala.
f. Periventricular Regions: Treating multiple sclerosis by accessing periventricular white matter lesions.
g. Occipital Arteries: Providing therapies for visual cortex-related disorders.
h. Brainstem Vessels: Targeting the brainstem for conditions affecting autonomic functions.

The targets in different cerebral hemispheres can be accessed through different routes. For example, a route of the microcatheter from the femoral Artery to the hippocampal arteries may include the following: (a) Femoral Artery: The catheter is inserted into the femoral artery, which is located in the upper thigh; (b) External Iliac Artery: It then advances into the external iliac artery, moving upward toward the pelvis; (c) Common Iliac Artery: The catheter proceeds into the common iliac artery, formed by the convergence of the external and internal iliac arteries; (d) Abdominal Aorta: It enters the abdominal aorta, the main arterial conduit running along the spine; (e) Thoracic Aorta: The catheter continues upward into the thoracic aorta within the chest cavity; (f) Aortic Arch: Upon reaching the aortic arch, it navigates toward the arteries supplying the head and neck; (g) Common Carotid Artery: The catheter is guided into the left or right common carotid artery, which ascends alongside the neck; (h) Internal Carotid Artery: It then moves into the internal carotid artery, entering the cranial cavity to supply the brain; (i) Cerebral Arterial Circle (Circle of Willis): The catheter reaches the Circle of Willis, a ring-like arterial structure at the base of the brain that provides multiple pathways to cerebral regions; (j) Posterior Cerebral Artery: From the Circle of Willis, the catheter is directed into the posterior cerebral artery; (k) Hippocampal Arteries: Finally, it reaches the hippocampal arteries, small branches that supply blood to the hippocampus located in the temporal lobe.

To successfully navigate from the femoral artery to the hippocampal arteries, the microcatheter configured for PIRL laser pulse delivery and distal dispending of a pharmaceutical fluid may include the following properties: (a) Minimum Bend Radius: The catheter must accommodate bends with radii as small as 5 mm; (b) Catheter Tip Size: A microcatheter with an outer diameter of approximately 0.5 mm (1.5 Fr) is necessary to traverse the smallest cerebral vessels leading to the hippocampus; (c) Flexibility: the microcatheter must have sufficient flexibility to allow advancing of the fiber without buckling while also enabling steering capability. Likely different lengths of the catheter may require different flexibilities, for example, the portion near the distal tip could be more flexible to allow improved steering via tendons or other methods discussed here. This configuration can be beneficial in that the catheter can safely and effectively reach the target area for delivering the picosecond mid-infrared laser, facilitating minimally invasive access across the blood-brain barrier for therapeutic interventions.

A suitable microcatheter diameter and tip size may be determined by the diameter of the smallest vessels it must navigate. For example, the following examples provide suitable microcatheter diameters for navigation with various vessels: (a) Femoral Artery: Diameter of about 6-8 mm, accommodating larger guide catheters (up to 6-8 French); (b) Internal Carotid Artery: Diameter decreases to about 4-5 mm; (c) Middle Cerebral Artery (MCA): Further narrows to 2-3 mm; (d) Hippocampal Arteries: These are small perforating arteries with diameters ranging from 0.5 to 1 mm. For example, to reach the hippocampal arteries, a microcatheter with an outer diameter (OD) of 1.2-1.7 French (Fr) may be employed. Complementary guidewires/microwires with diameters of 0.25-0.36 mm can be used to navigate these small vessels.

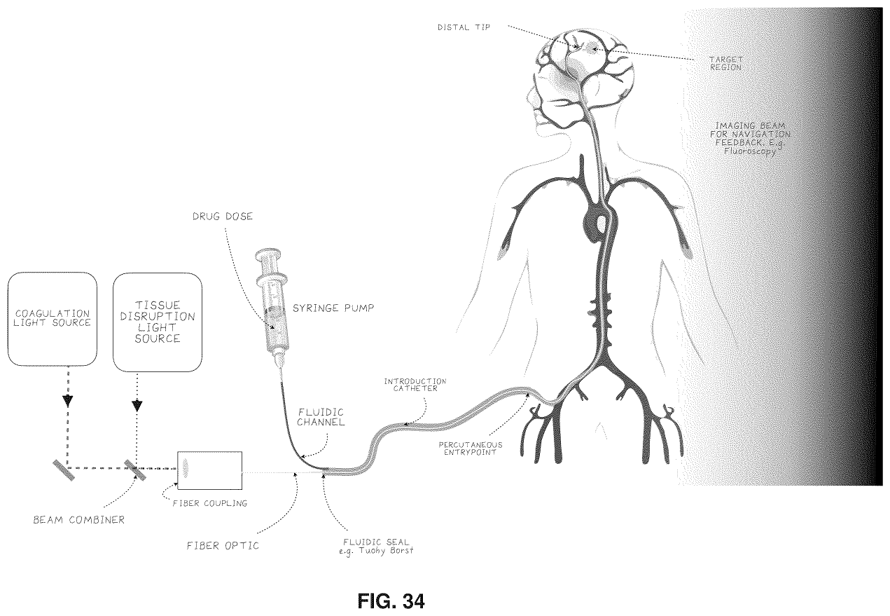

FIG. 34 shows an example catheter system for performing intravascular and transvascular intracranial delivery of a pharmaceutical to an intracranial region beyond the blood-brain barrier, in the present example implementation, via a transfemoral path. The figure illustrates a percutaneous approach through the femoral or radial arteries utilizing microcatheter including small fiber optics to deliver a picosecond mid-infrared laser into the cerebral vasculature to create micro-perforations in vascular tissue, thereby enabling the passage of therapeutic small molecules into targeted brain regions. The microperforations being either self sealing, or sealed via thermal coagulation using the same laser or a second coagulation source.

As illustrated in FIG. 34, after access to the femoral artery has been established, the catheter enters the femoral artery with advancement to the iliac artery, up the abdominal aorta, through the aortic arch to one of the carotid arteries, all the way to one of the cerebral arteries closest to the target area of the brain. Advancement of the catheter can be visually monitored and guided by imaging methods such as fluoroscopy. Telescoping catheters may be used, where the largest and outermost goes from the femoral artery to the carotid artery in the neck; through this, a smaller and longer catheter is advanced to the base of the skull, and finally, the smallest innermost catheter containing the combined fiber optic and fluidic channel, is advanced to the brain artery of interest, where the delivery of PIRL laser pulses can be employed to disrupt the vessel wall to a facilitate intracranial delivery of a liquid pharmaceutical. While FIG. 34 illustrates an example microcatheter embodiment based on the sheath implementation of the distal portion of the elongate conduit of the intracorporeal laser pulse delivery assembly illustrated in FIG. 12A, it will be understood that any of the present example embodiments, and/or variations and adaptions thereof, may be employed to configure the fluidic delivery structure of the microcatheter.

The present innovative intravascular PIRL and pharmaceutical delivery approach allows for precise targeting of specific brain regions through the vascular network, potentially revolutionizing the treatment of various neurological conditions by facilitating the delivery of therapeutic agents across the blood-brain barrier. Moreover, while the present example intravascular systems, devices and methods are described with reference to accessing intracranial targets, it will be understood that the systems, devices and methods described herein may be readily adapted to achieve vascular access to organs other than the brain, for example the liver and the pancreas.

One example implementation of an intravascular system may be provided as follows. Catheter Selection: A microcatheter of 1.5 Fr (0.5 mm OD) would be suitable for accessing the hippocampal arteries without causing vessel trauma. The catheter should be made of materials like braided stainless steel or nitinol to provide the necessary flexibility and kink resistance while accommodating a fiber optic for laser mediated perforation. A hydrophilic surface reduces friction, facilitating smoother navigation through tortuous paths. Since the cerebral arteries, especially the small perforators like the lenticulostriate arteries, have tight curves with bend radii as small as 3 mm or even less, some practical requirements can be defined for the fiber optics size and bend radius. Assuming that the minimum allowable bend radius of a particular fiber optic waveguide is roughly proportional to the fiber's diameter. One can select a suitable diameter, for example, low OH Si glass can support a minimum bend radius that is approximately 20 times the fiber diameter.

To design the largest core size low-OH step-index (SI) fiber suitable for bending to a radius of 3-5 mm in catheter applications, a fiber with a 200 μm core diameter and a high numerical aperture (NA) of 0.37 is ideal. This large core allows for maximum light transmission and reduced bend losses, while the high NA ensures efficient light confinement despite tight bends. The cladding diameter would be approximately 220 μm, and with protective coatings, the overall diameter could reach around 500 μm, fitting within standard catheter sizes. The low-OH silica material minimizes attenuation at mid-IR wavelengths such as 1.9 μm, and the fiber's mechanical robustness, enhanced by suitable coatings, provides the necessary flexibility and durability for safe and effective use in catheter-based procedures.

For higher transmission at longer mid-infrared wavelengths (around 3-5 μm), fluoride glass fibers like ZBLAN are considered, which, despite being more brittle than conventional silica fibers, offer the best balance between transmission properties and mechanical feasibility within specified constraints for delivering picosecond mid-IR lasers beyond 2.2 μm. Assuming a factor of 100 between the minimum bend radius and the core diameter, a bend radius of 5 mm corresponds to a 50 μm core diameter in ZBLAN fiber; from this core size, a cladding diameter of 70 μm and a total outer diameter with coating of ≤500 μm can be determined, achieving a minimum bend radius of less than 5 mm through design enhancements. Therefore, a ZBLAN fiber optimized for flexibility, with these specifications, is suitable for applications requiring tight bends and efficient mid-IR transmission within the specified constraints.

Energy Threshold goes with the square of the fiber output face diameter, for example, for a 200 μm fiber at 2950, <1 ns the threshold is 80 uJ. For a 50 μm diameter fiber, the energy threshold for tissue disruption would be 16× smaller. E.g. 5 uJ for 2950 nm, 500 ps. Operating above the threshold can be advantageous for efficient tissue disruption, eg. 2× or 4× threshold. 10 uJ and 20 uJ.

However, if the tip is polished at an angle, or tapered as a cone for example, to increase the surface are size, the energy would scale linearly with the new surface area at which the laser output occurs. It is critical that the intensity within the narrowest part of the fiber optic waveguide not exceed the optical damage threshold of the waveguide itself, while also being above the energy threshold for tissue disruption. For this reason, the very efficient tissue disruption mechanisms, e.g. impulsive heat deposition, provide novel regimes in which the tissue disruption threshold is far below the optical damage threshold of the waveguides that transmit well in the vibrational absorption bands of OH containing tissue.

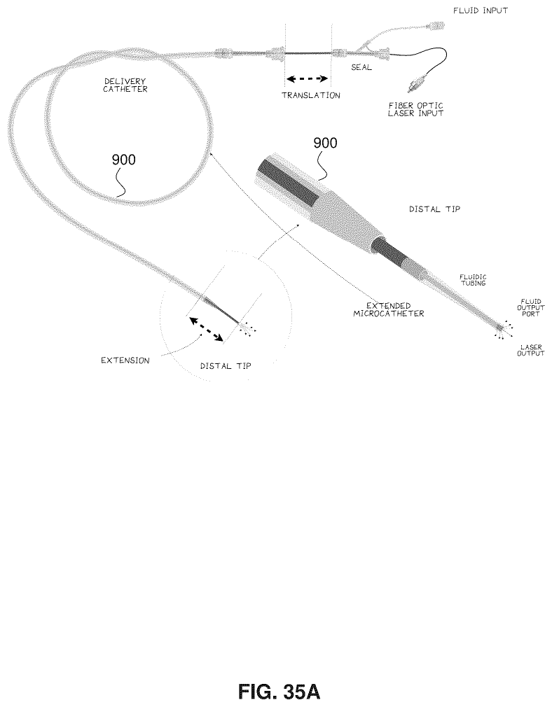

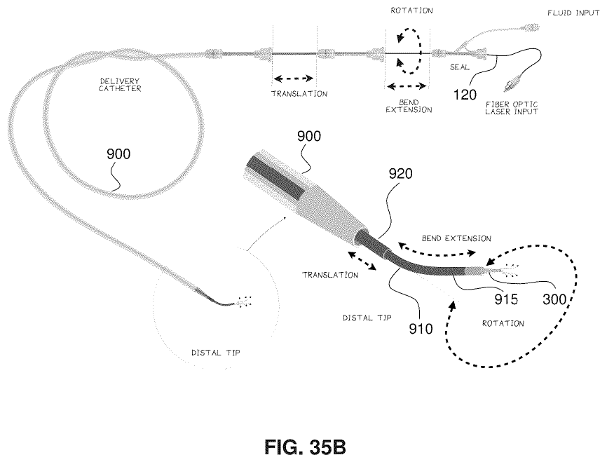

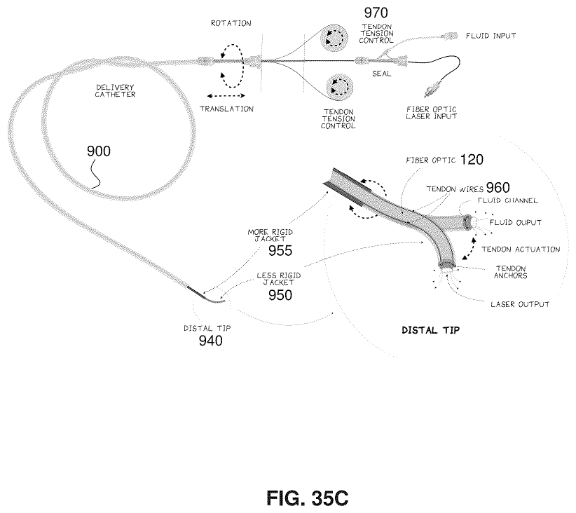

FIG. 35A illustrates an example of a microcatheter system that includes a delivery catheter 900 for navigating to the cranial vasculature and an extendable microcatheter that includes an optical fiber and a fluid delivery channel, with a distal fluid delivery region similar to that shown in FIG. 12A.

Likewise, FIG. 35B illustrates an example of a microcatheter system that includes a delivery catheter 900 for navigating to the cranial vasculature and an extendable, steerable microcatheter that includes an optical fiber, a fluid delivery channel, and a concentric-tube-based steering mechanism that includes an inner tube 910 houses the optical fiber 120 and the fluidic delivery path, from which the distal sheath 300 extends as in FIG. 12A, the inner tube 910 having a bent distal portion 915 and an outer tube 920 beyond which the inner tube is extendable, as illustrated in the figure.

FIG. 35C illustrates an example of another example of a microcatheter system that includes a delivery catheter 900 for navigating to the cranial vasculature and an extendable, steerable microcatheter that includes an optical fiber 120 and a fluid delivery channel which is steered by deforming the distal tip region 940 (having a less rigid outer jacket 950 than a more rigid proximal jacket 955) using tendons 960 connected to a proximal tendon tension controlling mechanism 970.

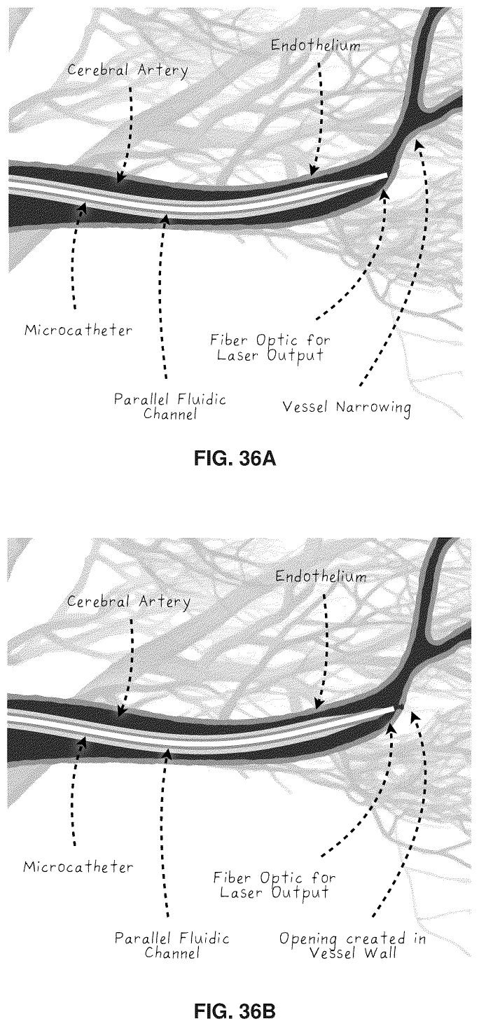

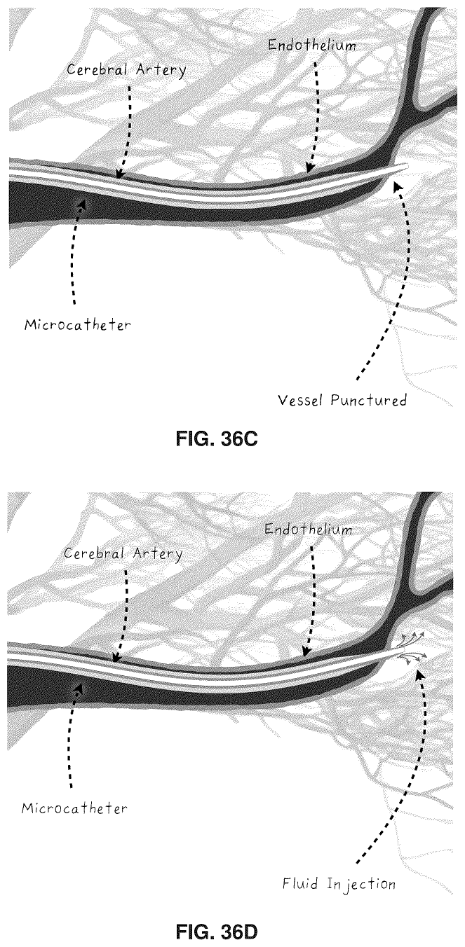

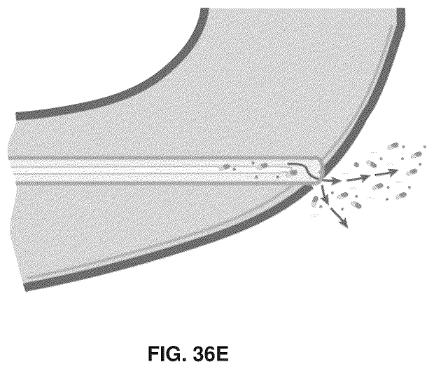

FIGS. 36A, 36B, 36C, 36D and 36E illustrate an example method in which the distal end of the microcatheter is positioned adjacent to a vessel bend and extended through the vessel wall while delivering PIRL laser pulses to facilitate disruption of the vessel wall, followed by dispensing of a pharmaceutical fluid beyond the vessel wall. As shown in the figures, a laser microcatheter that includes a fluidic channel is guided passively under fluoroscopy to the vessel closest to the target region, until the catheter hits a vessel wall that is too small to accommodate the catheter diameter. At this position the laser is fired to puncture the vessel wall and pharmaceutical fluid is delivered directly into the interstitial region of the brain.

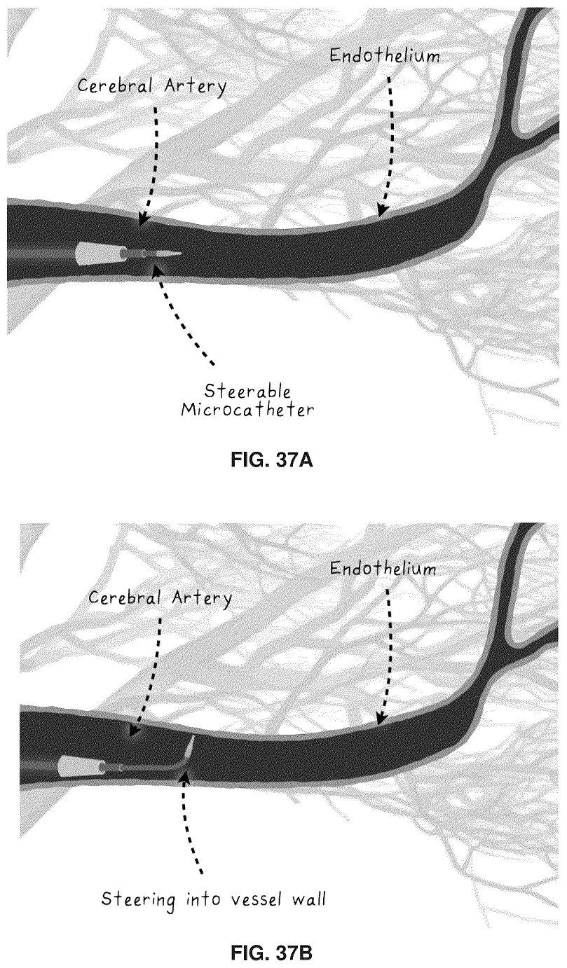

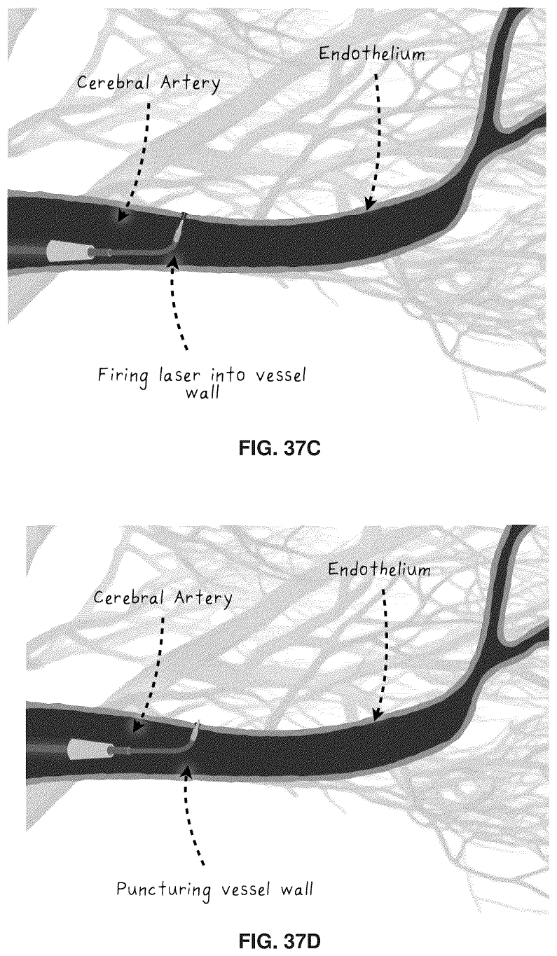

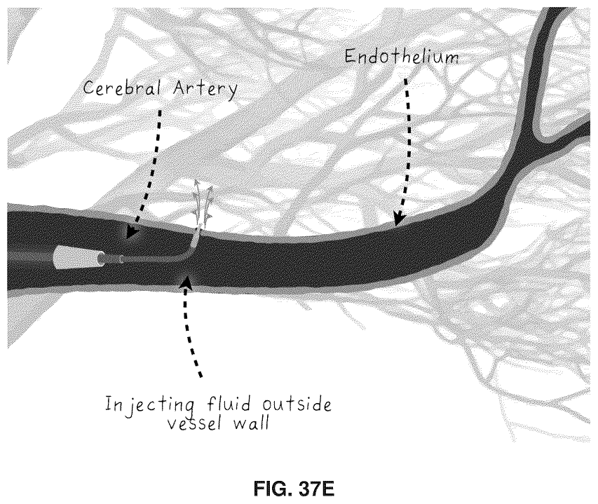

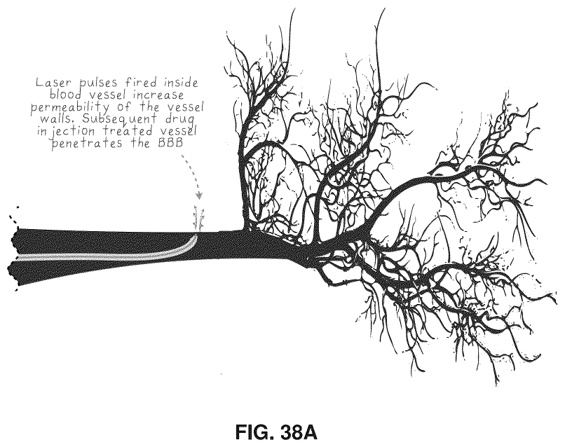

FIGS. 37A, 37B, 37C, 37D and 37E illustrate an example method in which the distal end of the microcatheter is steered adjacent to a vessel wall and extended through the vessel wall while delivering PIRL laser pulses to facilitate disruption of the vessel wall, followed by dispensing of a pharmaceutical fluid beyond the vessel wall. As shown in the figures, a laser microcatheter is actively guided using a steering mechanism to the target region of interest and then using the steering mechanism, it is positioned where the laser is fired to puncture the vessel wall.

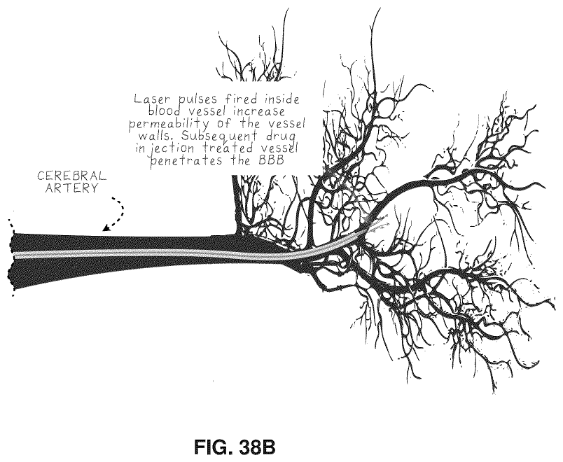

FIGS. 38A and 38B illustrate an example method in which the distal end of the microcatheter is adjacent to a vessel wall and PIRL laser pulses are delivered to facilitate disruption of the vessel wall, followed by dispensing of a pharmaceutical fluid proximal to the disrupted vessel wall, such that the pharmaceutical fluid extends through the disrupted vessel wall. As shown in the figures, a laser microcatheter is guided to the wall of a blood vessel, where PIRL laser pulses are fired to increase permeability of blood vessel walls via thermal or photomechanical/photoacoustic (ultrasonic) effects, so that the pharmaceutical fluid is delivered nearby the vessel wall can penetrate the blood-brain barrier without puncture of the vessel wall.

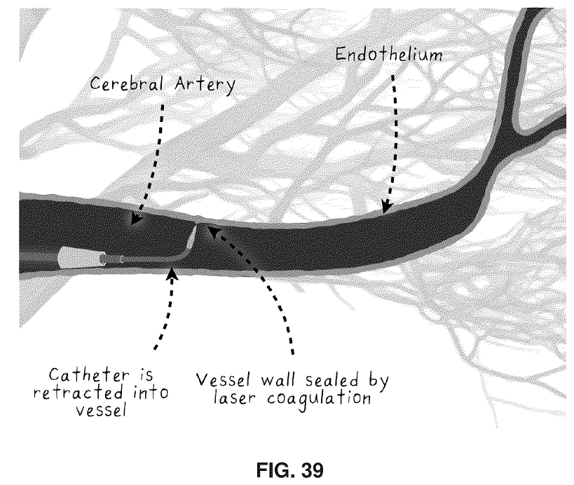

FIG. 39 illustrates a method of vascular wall coagulation via the delivery of additional non-PIRL laser pulses. For example, as described above in the example case of transvascular passage, blood leakage can be mitigated and as an additional safety measure by including another laser to selective coagulate the blood exactly at the aperture or leakage site. This may be achieved, for example, using green laser light, as the liquid will not transmit infrared, with the green laser inducing coagulation over a larger volume than the infrared light would and provides enough coagulated protein or “putty” to act as patch to seal off any leakage.

The optical fiber, located adjacent to the vessel opening, may be employed to close the hole (induce vascular repair) to fully prevent blood or other fluid leakage by cumulative heating to locally coagulate blood or other proteinaceous matter to seal the vessel. Air may be injected through the microcatheter fluid channel to locally cauterize, burn tissue, for a hard seal to prevent any occurrence of leakage at the opening. As shown in the figure, green laser light may be employed upon withdrawal of the microcatheter to selectively excite blood thereby inducing coagulation and/or cauterization to stop the bleeding during retraction once the vessel has been penetrated and fluids delivered.

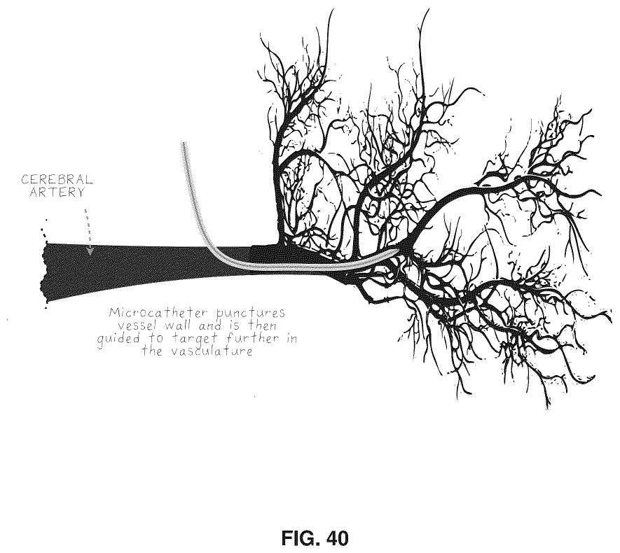

FIG. 40, shows a stereotaxic guided system for direct access to the cerebral arteries through the skull. This is an alternative path where imaging and stereotaxic guidance is used to navigate a microcatheter into a specific surface cerebral arteries without travelling through a long section of the vasculature. This is accomplished by creating an entry point in the skull in close proximity to an entry point in a cerebral artery close to the surface of the brain, and, for example, and entering through the skull with the microcatheter retracted within a microcannula, and employing the delivery of PIRL laser pulses to facilitate entry of the microcannula through the skull and into a cranial vessel, followed by extension of the microcatheter to a desired intravascular location for subsequent PIRL-based disruption of the vessel wall and delivery of a pharmaceutical fluid. Examples include the branches of the major cerebral arteries: the anterior cerebral artery (ACA), middle cerebral artery (MCA), and posterior cerebral artery (PCA), which run along the surface of the brain in the subarachnoid space (between the arachnoid mater and pia mater) and supply blood to the outer layers of the brain. It is possible to access the cerebral arteries on the outer surface of the brain through a small hole in the skull, a procedure known as a burr hole or craniotomy. In this case, the diameter of the craniotomy could be sub-mm to accommodate the microcatheter. In some cases the microcraniotomy could be created with the same laser mechanism for minimal invasive entry to the brain.

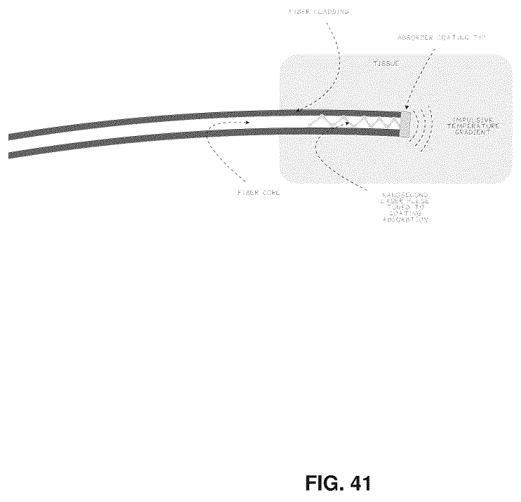

Referring now to FIG. 41, this figure introduces an alternative delivery mechanism for a laser-based system employing impulsive heat deposition to disrupt tissue, wherein a thin layer is deposited on the distal tip of a fiber optic. In this novel approach, a nanosecond pulsed laser, operating in the mid-IR, near-IR, or visible spectrum, is used to irradiate the fiber optic tip coated with a mechanically strong, highly absorptive material. Unlike the previously described mid-IR laser for impulsive heat deposition, which relies on lasers tuned to the vibrational modes of water or tissue constituents for absorption, the coating on the fiber tip is specifically designed to absorb the incident laser energy efficiently. Upon irradiation, the rapid temperature rise in the coating induces impulsive heating, which vaporizes the adjacent tissue. This method provides a highly localized and effective tissue ablation technique, offering enhanced precision and flexibility in laser-tissue interaction applications.

A wide variety of materials can be coated onto the end of a fiber optic to create a thin layer that efficiently absorbs light energy while possessing high heat capacity. Gold (Au) and gold nanoparticles (AuNPs) are commonly used due to their excellent thermal conductivity and plasmonic properties, allowing strong light absorption. Carbon nanotubes (CNTs) and graphene are both highly effective, with graphene offering exceptional light absorption and thermal dissipation in a single atomic layer. Black silicon, a nanostructured form of silicon, absorbs light across a wide wavelength range, while platinum (Pt) and silver nanoparticles (AgNPs) offer good light absorption, with silver also providing plasmonic benefits. Copper oxide (CuO) is notable for its high visible light absorption, and titanium nitride (TiN) has plasmonic properties combined with high thermal stability.

Other non-limiting examples of optically absorbing coating materials include the Molybdenum disulfide (MoS₂), which is effective in the visible and infrared spectrum, and silicon carbide (SiC), known for its high thermal conductivity and efficient light absorption. Tungsten (W) is ideal for high-temperature applications due to its high heat capacity and ability to absorb light well. Iron oxide nanoparticles (Fe₃O₄) offer good thermal properties and light absorption, while vanadium dioxide (VO₂) is a thermochromic material with efficient light absorption at elevated temperatures. Zinc oxide (ZnO), particularly in the UV range, and aluminum (Al), in the form of nanoparticles, are also effective light absorbers, with aluminum exhibiting strong plasmonic behavior similar to gold and silver.

The coating should be provided with sufficient thermal mass to store enough energy upon absorption of a single laser pulse or a burst of pulses, without exceeding the melting point of the material, to cause through thermal transfer of the distal surface of the coating into adjacent tissue, and the laser pulse or burst of pulses is provided with sufficient energy such that a volume of the tissue local to the tip is vaporized or emulsified into a liquid phase.

As an example method to determine the requirements of the coated tip that can transfer sufficient energy to cause micro disruption of tissue, one can begin by looking at the minimum pulse energy requirements for vapourization of water using a ˜3 μm wavelength sub-nanosecond laser. The laser mechanism energizes a volume of water approximately the size of a disc with 1 μm thickness and diameter of the fiber optic until it is vaporized. The necessary pulse energy conditions for a given fiber tip can be calculated assuming that all laser energy is absorbed evenly by a specified water volume, and the laser pulse duration is extremely short (nanoseconds or less), allowing one to neglect heat transfer (thermal confinement). the minimum energy required for vaporization of water at the tip of fiber of diameter D, given:

$${Q}_{total} ( D ) = \frac{ρ π {D}^{2} h}{4} ( {c}_{p} Δ T + {L}_{v} ) ,$$

where ρ is the density of water (typically 1000 kg/m3), h is the thickness of the water layer, determined by the absorption of the specific laser wavelength (in this case close to 2.95 μm), Cp is the specific heat cavity of water 4180 J/(kg*K) and ΔT is the temperature change required to reach boiling point (approximately 100 C-25 C) and Lv is the latent heat of vaporization of water 2.26×106 J/kg.

For example, a PIRL with wavelength ˜2.9 μm and a fiber optic with a 200 μm core at the tip. The total energy required to vaporize a 1 μm thick is Qtotal(D)=81.5 uJ. Now however, considering the situation where there is a coating material which absorbs the laser energy at the fiber tip instead of the water so that the fiber optic can be coupled with a short pulsed laser, approximately 1=100 nanoseconds, with wavelengths that do not absorb well in tissue, but absorbs well in the chosen coating material. In this case the coating temperature increases rapidly due to the short laser pulse, and heat is transferred into the water that is in contact with the tip. The minimum pulse energy required to vaporize the same volume of water near the tip would be the same as the example above, with the exception of thermal transfer inefficiencies. However it is important to consider the thermal capacity and energy storage of the coating. In other words, the coating must be thick enough to store sufficient energy to transfer Qtotal(D) to the adjacent volume of water. The following limitations must be considered. Firstly, the maximum temperature of the coating must not exceed its melting point. (e.g. for gold 1064 degC). Yet for sufficient energy transfer there must be at least sufficient mass of the coating.

The energy stored in the coating volume can be calculated as Emax=ρgold×Cp×Vgold×ΔT, where ρgold the density of gold is 19,320 kg/m\hat{ }3, Cp is the specific heat capacity of gold 129 J/(kg*K) and Vgold is the volume of the gold disc of radius r and thickness t and ΔT is the temperature rise in Kelvin up to the melting point ˜1044K.

Thus as a function of thickness, one can calculate for a gold coating

Emax=Emax=19300(kg/m3)*129J/(kg*K)*pi*(100E−6)2*1044*t=81.6 J/m*thickness

For a 100 nm thick coating the max energy storage is 8.16 uJ. Thus the coating thickness should be larger than approximately 1 μm to store the necessary energy to vaporize the same layer as water assuming complete energy transfer, uniform heating and instantaneous heat transfer times. In other words, a 1 μm thick, 200 μm diameter gold disc that is instantaneously heated to its melting temperature (1064° C.) by a short pulsed laser, and then brought into contact with water at room temperature (25° C.), can impart into the water only the maximum energy stored in the gold disc before melting-approximately 81.2 uJ in this case. Assuming no energy lost to heat transfer, the energy is sufficient in this example to vaporize a 1 μm thick layer of water over the area of the gold disc as the energy stored in the gold matches the energy required for this phase change when the enthalpy of vaporization is correctly included.

However, heat transfer also increases the requirements of the laser pulse energy and must be taken into account. One can assume that heat loss will result in some lack of efficiency compared to PIRL-based vaporization, but so long as the pulse energy is sufficient to vaporize a small volume of water at the tip, and also below the optical damage threshold of the fiber optic or the bond between the gold layer and the fiber surface, a minimum pulse energy can be calculated based on thermal transfer dynamics.

### EXAMPLES

The following examples are presented to enable those skilled in the art to understand and to practice embodiments of the present disclosure. They should not be considered as a limitation on the scope of the disclosure, but merely as being illustrative and representative thereof.

### Example 1: Demonstration of Ultrasound Detection of Optical Fiber Tip Location Via Detection of Photoacoustic Signals During Delivery of PIRL Pulses

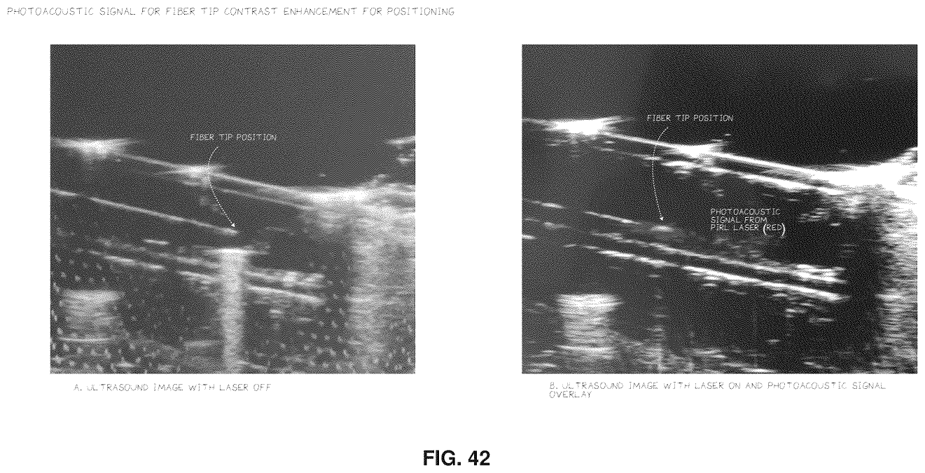

An optical fiber was imaged using a vivo 3100 high resolution ultrasound imaging system, as shown in FIG. 42. The left image shows the ultrasound image prior to the delivery of PIRL pulses by the optical fiber. The right image was obtained while the optical fiber was emitting PIRL pulses, and the image clearly shows the photoacoustic signal associated with PIRL-induced cavitation beyond the distal end of the optical fiber. The laser output was synchronized to the imaging system for timing gating of the imaging ultrasound signal and the photo acoustic signal generated by the laser firing in the liquid.

### Example 2: Laser Systems for PIRL Pulse Generation and Delivery

Non-limiting examples of suitable laser systems for the generation of PIRL laser pulses include a near-IR pumped optical parametric amplifier (e.g. emitting pulses with a duration in the hundreds of ps, such as 500 ps) tuned to a wavelength of approximately 2.95 μm, operating, for example, between 0.05-10 kHz with a pulse fluence greater than 0.1 mJ/pulse, delivered on target, for example, through a sapphire fiber optic (e.g. having a core diameter of 200 μm where the parametric amplifier could include one or more bulk nonlinear crystal, such as Potassium Titanyl Arsenate (KTIOAsO4), or a periodically poled nonlinear crystal such as periodically poled such as PPKTP and is either CW or pulse seeded at the signal ˜1.6 μm or idler wavelength 2.95 or seeded by a cascaded parametric process, such as optical parametric generation (OPG) at either wavelength that using a portion of the same pump laser, and pumped with an amplified near-IR picosecond laser such as a Nd:YAG or Nd:YLF regenerative amplifier or Master Oscillator Power Amplifier; and a Cr:ZnSe gain-switched laser, emitting ns pulses (e.g. 1.5 ns), tuned to a wavelength of approximately 2.7 μm, operating, for example, between 0.05-10 kHz, with a pulse fluence greater than >0.1 mJ/pulse output from the fiber optic system, or alternatively an all fiber or hybrid laser system in which a fiber amplifier consisting of a gain material such as Erbium doped ZBLAN fiber is used to amplify pulses from a fiber laser, pulsed or modulated diode laser, or Q-switched microchip lasers and amplifies them to an output with wavelength approximately 2.8 μm, operating, for example, between 1-10 kHz, with a pulse energy greater than >0.1 mJ/pulse either coupled directly into a mid-IR transmitting output fiber or through free space into a beam delivery fiber. In general, the pulse energy requirement regardless of the laser architecture is determined by the intensity at the output of whatever size fiber optic is chosen and the mechanical properties of the target material, from 50 μm to 1 mm diameters intensity must be >0.25 J/cm\hat{ }2. The average power is a function of the maximum advancement speed and the thermal properties of the target material, so >100 Hz for >0.5 mm/s speeds would be typical.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

1. A system capable of performing laser-assisted tissue disruption, the system comprising:
a pulsed infrared laser source configured to generate infrared laser pulses;
an intracorporeal laser pulse delivery assembly comprising:
an elongate conduit comprising an inner lumen and an elongate distal portion configured for intracorporeal insertion and having a diameter of less than 1 mm, said elongate distal portion having a distal opening in fluid communication with said inner lumen, said elongate conduit further comprising a proximal port, said proximal port being in fluidic communication with said inner lumen;
an optical fiber having a proximal end coupled to said pulsed infrared laser source such that the infrared laser pulses are delivered through said optical fiber, said optical fiber extending through said elongate conduit; and
a pump mechanism in fluid communication with said proximal port;
said pulsed infrared laser source being configured such that the infrared laser pulses have:
a wavelength selected such that absorption by a laser-irradiated tissue volume residing beyond a distal end of said optical fiber, following intracorporeal insertion of said elongate distal portion, is predominantly due to excitation of vibrational modes of one or more constituents of the laser-irradiated tissue volume;
a pulse duration that is shorter than a first time duration required for thermal diffusion out of the laser-irradiated tissue volume and shorter than a second time duration required for a thermally driven expansion of the laser-irradiated tissue volume;
a pulse fluence and the pulse duration resulting in a peak pulse intensity below a threshold for ionization-driven tissue disruption to occur within the laser-irradiated tissue volume; and
wherein the pulse fluence is sufficiently high to cause local tissue disruption and liquification of the laser-irradiated tissue volume.

2. The system according to claim 1 wherein said elongate conduit further comprises a proximal valve, and wherein said optical fiber passes through said proximal valve into said elongate conduit and extends through said elongate conduit, said proximal valve being configurable to form a fluidic seal with said optical fiber while permitting longitudinal movement of said optical fiber, wherein an outer diameter of said optical fiber is less than a diameter of said inner lumen, such that said proximal port is in fluid communication with said distal opening;
wherein said intracorporeal laser pulse delivery assembly further comprises a distal beam expansion optical element coupled to said distal end of said optical fiber, such that said distal beam expansion optical element is mechanically supported by said optical fiber and resides beyond said distal opening of said elongate conduit, and such that the infrared laser pulses propagate from said optical fiber into said distal beam expansion optical element, and expand within said distal beam expansion optical element to substantially fill a distal forward-facing surface of said distal beam expansion optical element;
said distal beam expansion optical element having a proximal portion configured to close and seal said distal opening of said elongate conduit when said optical fiber is retracted through said proximal valve, and configured to facilitate fluidic communication between said inner lumen and an external region beyond said distal opening when said optical fiber is extended through said proximal valve and said distal beam expansion optical element is moved distalward relative to said distal opening, said distal beam expansion optical element and said distal opening thereby forming a distal valve.

3. The system according to claim 2 wherein said distal forward-facing surface is curved to facilitate reduction in a frictional force during intracorporeal insertion of said elongate distal portion.

4. The system according to claim 1 wherein said intracorporeal laser pulse delivery assembly is further configured such that:
said elongate conduit comprises a proximal seal;
said elongate distal portion is a sheath extending distalward from a proximal portion of said elongate conduit, said inner lumen extending through said sheath to a distal opening of said sheath;
said optical fiber passes through said proximal seal, into said proximal portion of said elongate conduit, and extends through said sheath, such that said distal end of said optical fiber resides at or distally adjacent to a distal end of said sheath; and
said sheath has a tapered profile such that a distal portion of said sheath is closer to an outer surface of said optical fiber than a proximal portion of said sheath;
wherein said pump mechanism is operable to dispense a fluid from said distal end of said sheath.

5. The system according to claim 4 wherein said distal portion of said sheath contacts and applies a passive compressive force to said outer surface of said optical fiber, thereby forming a distal valve that is passively closed via the passive compressive force, and wherein said pump mechanism is configured to apply sufficient pressure to overcome the passive compressive force to facilitate dispensing of the fluid through said distal valve.

6. The system according to claim 1 wherein said intracorporeal laser pulse delivery assembly further comprises:
a rigid body;
a syringe comprising a syringe barrel and a movable plunger having a plunger seal, said syringe barrel and said plunger seal defining an inner chamber;
wherein a proximal portion of said optical fiber is supported by a rigid elongate support, said rigid elongate support being rigidly supported relative to said syringe barrel by said rigid body, said rigid elongate support passing through said plunger seal, into said inner chamber, wherein said plunger seal is configured to form a fluidic seal with an outer surface of said rigid elongate support and an inner surface of said syringe barrel while said movable plunger is moved relative to said rigid elongate support; and
wherein said elongate conduit extends from a distal end of said syringe barrel, such that said inner chamber is in fluid communication with said inner lumen of said elongate conduit through said proximal port of said elongate conduit, and wherein said optical fiber extends from a distal end of said rigid elongate support, through said inner chamber, and into said elongate conduit;
wherein said movable plunger, when actuated, is capable of dispensing a fluid from said distal end of said elongate distal portion of said elongate conduit.

7. The system according to claim 1 wherein said elongate distal portion is a microcannula suitable for incorporeal insertion.

8. The system according to claim 1 wherein said elongate distal portion is a microcatheter suitable for intravascular insertion and navigation.

9. The system according to claim 8 wherein said intracorporeal laser pulse delivery assembly further comprises a microcannula, said elongate distal portion of said elongate conduit being retractable within said microcannula to facilitate intracorporeal insertion of said microcannula during delivery of the infrared laser pulses.

10. The system according to claim 9 further comprising a steering mechanism for steering said microcatheter when said microcatheter is extended from said microcannula.

11. The system according to claim 1 wherein said pump mechanism is in fluid communication with a pharmaceutical fluid, and wherein said pump mechanism is controllable to facilitate direct injection of the pharmaceutical fluid after performing local tissue disruption and liquification of a laser-irradiated tissue volume.

12. A method of delivering a pharmaceutical fluid to an intracorporeal target site, the method comprising:
providing the system according to claim 1 such that the pharmaceutical fluid resides within at least a distal portion of said inner lumen;
controlling said pulsed infrared laser source to perform local tissue disruption and liquification while positioning said distal end of said elongate distal portion proximal to or within the intracorporeal target site; and
controlling said pump mechanism to dispense the pharmaceutical fluid from said distal opening.

13. The method according to claim 12 wherein the intracorporeal target site resides within a brain of a subject, and wherein said elongate distal portion is a microcannula suitable for incorporeal insertion, and wherein said pulsed infrared laser source is controlled to perform local tissue disruption and liquification to facilitate insertion of said microcannula through a skull of a subject and positioning of a distal end of said microcannula within the brain of the subject.

14. The method according to claim 13 wherein said pulsed infrared laser source is controlled to perform local tissue disruption and liquification while passing through vessel walls of a cranial vessel as said distal end of said microcannula is directed to the intracorporeal target site within the brain.

15. The method according to claim 12 wherein said elongate distal portion is a microcatheter suitable for intravascular insertion and navigation, and wherein positioning said distal end of said microcatheter proximal to or within the intracorporeal target site comprises positioning a distal end of said microcatheter is adjacent to a vessel wall of a cranial vessel within a brain and controlling said pulsed infrared laser source to perform local tissue disruption and liquification of the vessel wall.

16. The method according to claim 15 further comprising extending said distal end of said elongate distal portion through the vessel wall to position said distal end of said microcatheter proximal to or within the intracorporeal target site.

17. The method according to claim 15 wherein said distal end of said elongate distal portion is positioned to adjacent to the vessel wall at a vascular bend in the absence of steering of said elongate distal portion.

18. The method according to claim 15 wherein said elongate distal portion is steerable according to a steering mechanism, and wherein said distal end of said elongate distal portion is steered to position said distal end of said elongate distal portion adjacent to the vessel wall.

19. The method according to claim 15 wherein a delivery catheter is employed to facilitate positioning of said distal end of said elongate distal portion within the cranial vessel.

20. The method according to claim 15 wherein said intracorporeal laser pulse delivery assembly further comprises a microcannula, said elongate distal portion of said elongate conduit being retractable within said microcannula, and said microcatheter is inserted to the cranial vessel by:
retracting said microcatheter within said microcannula, such that said distal end of said optical fiber resides at or distally adjacent to a distal end of said microcannula;
while delivering the infrared laser pulses, performing intracranial insertion of said microcannula and positioning said distal end of said microcannula within the cranial vessel; and
extending said microcatheter from said microcannula to position a distal end of said microcatheter adjacent to the vessel wall.

21. The method according to claim 15 wherein the vessel wall comprises a blood-brain barrier.

22. A method of delivering a pharmaceutical fluid to an intracorporeal target site, the method comprising:
providing the system according to claim 2 such that the pharmaceutical fluid resides within at least a distal portion of said inner lumen;
with said distal valve residing in a closed state, controlling said pulsed infrared laser source to perform local tissue disruption and liquification while positioning said distal end of said elongate distal portion proximal to or within the intracorporeal target site;
extending said optical fiber to move said distal beam expansion optical element away from contact with said distal opening to open said distal valve; and
controlling said pump mechanism to dispense the pharmaceutical fluid from said distal opening.

23. The method according to claim 22 wherein closure of said distal valve is facilitated, at least in part, by the application of a negative pressure by said pump mechanism to said inner lumen.

24. The method according to claim 22 wherein opening of said distal valve is facilitated, at least in part, by the application of a positive pressure by said pump mechanism to said inner lumen.

25. A method of performing biopsy at an intracorporeal target site, the method comprising:
providing the system according to claim 2;
retracting said optical fiber to contact said distal beam expansion optical element with said distal opening and close said distal valve;
controlling said pulsed infrared laser source to perform local tissue disruption and liquification while inserting said elongate distal portion into the body and positioning said distal end of said elongate distal portion proximal to or within the intracorporeal target site;
extending said optical fiber to extend said distal beam expansion optical element away from contact with said distal opening and open said distal valve; and
controlling said pump mechanism to aspirate tissue residing proximal to said distal opening.

26. The method according to claim 25 wherein closure of said distal valve is facilitated, at least in part, by the application of a negative pressure by said pump mechanism to said inner lumen.

27. The method according to claim 25 wherein said proximal valve is capable of locking a position of said optical fiber relative to said elongate conduit, the method further comprising, actuating said proximal valve to lock the position of said optical fiber relative to said elongate conduit to maintain said distal valve in an open configuration during aspiration of the tissue.

28. A system capable of performing laser-assisted thermal tissue disruption, the system comprising:
a pulsed laser source configured to generate a laser pulse or a burst of laser pulses;
an optical fiber having a proximal end coupled to said pulsed laser source;
an optically absorbing material coated on a distal end of said optical fiber;
wherein the laser pulse or a burst of laser pulses is delivered from said pulsed laser source with an energy such that said optically absorbing material is heated to a temperature between below its melting point over a time duration that is shorter a thermal diffusion timescale of said optically absorbing material when said distal end of said optical fiber is placed in water;
wherein the laser pulse or a burst of laser pulses is provided with sufficient energy to cause vaporization of a volume of water residing adjacent to said distal end of said optical fiber; and
wherein said optically absorbing material is provided with a sufficient thickness to facilitate absorption of the laser pulse or a burst of laser pulses.

29. The system according to claim 28 wherein said optically absorbing material is selected such that at least a portion of said optically absorbing material remains adhered to said distal end of said optical fiber after delivery of the laser pulse or a burst of laser pulses.

30. A method of performing laser-assisted tissue disruption, the method comprising:
providing the system according to claim 28;
inserting said optical fiber into a subject and positioning said distal end of said optical fiber proximal to or within an intracorporeal target site; and
controlling said pulsed laser source to deliver the laser pulse or a burst of laser pulses, such that a tissue volume residing proximal to said distal end of said optical fiber is thermally vaporized or emulsified into a liquid phase.
